(19) United States
(12) Patent Application Publication
(10) Pub. No.: US 2024/0252486 A1
(43) Pub. Date: Aug. 1, 2024

(54) ALGOSTATIC AGENTS FOR PREVENTING TRANSITION FROM ACUTE TO CHRONIC PAIN

(71) Applicant: The Regents of the University of California, Oakland, CA (US)

(72) Inventors: Daniele Piomelli, Irvine, CA (US); Yannick Fotio, Irvine, CA (US)

(21) Appl. No.: 18/283,395

(22) PCT Filed: Mar. 22, 2022
(86) PCT No.: PCT/US2022/021339

§ 371 Date: Sep. 21, 2023

Related U.S. Application Data
(60) Provisional application No. 63/166,134, filed on Mar. 25, 2021.

(51) Int. Cl. A61K 31/496; A61K 31/164; A61K 31/196; A61K 31/222; A61K 31/337; A61K 31/55; A61K 45/06; A61P 29/00

(52) U.S. Cl. CPC A61K 31/496; A61K 31/164; A61K 31/196; A61K 31/222; A61K 31/337; A61K 31/55; A61K 45/06; A61P 29/00

(57) ABSTRACT

Disclosed herein, inter alia, are methods of stimulating mitochondrial respiration and methods of treating pain.

### CROSS-REFERENCES TO RELATED APPLICATIONS

This application claims the benefit of U.S. Provisional Application No. 63/166,134, filed Mar. 25, 2021, which is incorporated herein by reference in its entirety and for all purposes.

### BACKGROUND

More than 1.5 billion people worldwide suffer from chronic pain, which often starts after an acute pain episode. The sequence of molecular events that lead to pain chronicity is still largely unknown but filling this gap is necessary to identify control nodes that might be targeted by disease-modifying therapies. Disclosed herein, inter alia, are solutions to these and other problems in the art.

### BRIEF SUMMARY

In an aspect is provided a method of stimulating mitochondrial respiration in a nerve cell, including administering an agent to said nerve cell, wherein the agent is an NAAA (N-acylethanolamine acid amidase) inhibitor, FAAH (fatty acid amide hydrolase) inhibitor, PPARα (peroxisome proliferator-activated receptor-α) agonist, PEA (palmitoylethanolamide), acetyl-L-carnitine, α-lipoic acid, or olesoxime.

In an aspect is provided a method of treating pain in a subject in need thereof, the method including administering an effective amount of an agent to said patient, wherein said agent is administered between about 1 day and 7 days after a traumatic pain event and wherein said agent is an NAAA (N-acylethanolamine acid amidase) inhibitor, FAAH (fatty acid amide hydrolase) inhibitor, PPARα (peroxisome proliferator-activated receptor-α) agonist, PEA (palmitoylethanolamide), acetyl-L-carnitine, α-lipoic acid, or olesoxime.

In an aspect is provided a method of preventing chronic pain in a subject with acute pain, the method including administering an effective amount of an agent to the subject, wherein the agent is administered between about 1 day and 7 days after a traumatic pain event and wherein said agent is an NAAA (N-acylethanolamine acid amidase) inhibitor, FAAH (fatty acid amide hydrolase) inhibitor, PPARα (peroxisome proliferator-activated receptor-α) agonist, PEA (palmitoylethanolamide), acetyl-L-carnitine, α-lipoic acid, or olesoxime.

In an aspect is provided a method of preventing chronic pain in a subject, the method including administering perioperatively an effective amount of an agent to the subject, wherein the agent is a NAAA inhibitor, FAAH inhibitor, PPARα agonist, PEA, acetyl-L-carnitine, α-lipoic acid, or olesoxime.

In an aspect is provided a method of preventing chronic pain in a cancer patient, the method including administering an effective amount of an agent to the cancer patient, wherein said agent is a NAAA inhibitor, FAAH inhibitor, PPARα agonist, PEA, acetyl-L-carnitine, α-lipoic acid, or olesoxime.

In an aspect is provided a method of preventing chronic pain in a diabetic patient, the method including administering an effective amount of an agent to the diabetic patient, wherein said agent is a NAAA inhibitor, FAAH inhibitor, PPARα agonist, PEA, acetyl-L-carnitine, α-lipoic acid, or olesoxime.

### BRIEF DESCRIPTION OF THE DRAWINGS

FIGS. 1A-1I. Chemical end-organ damage produces long-lasting bilateral sensory abnormalities in vulnerable mice. FIG. 1A: Time-course of contralateral hyperalgesia assessed 3 h, 24 h, 48 h and 72 h after injection of saline (left, n=10) or 1% formalin (right, n=15) in male mice. FIG. 1B: Left, time-course of contralateral hyperalgesia (withdrawal latency, s) in mice given intraplantar injections of saline (Veh, open circles) or 1% formalin (Form, closed circles). Responses in formalin-treated mice did not distribute normally (K²=37.74; P<0.0001) and a K-means analysis parsed out two clusters, which were termed ‘vulnerable’ (Vuln) or ‘resilient’ (Res) based on their susceptibility to the lasting effects of formalin. Right, pie charts showing that the ratio of Vuln vs. Res mice remained constant over a 60-day period. PFD, post formalin day. FIG. 1C: Lack of correlation between immediate nocifensive response to formalin (0-10 min) and contralateral hyperalgesia assessed on PFD14. FIG. 1D: Lack of correlation between formalin-induced paw edema (A thickness, mm) and lasting contralateral hyperalgesia on PFD14. FIG. 1E: Left, time-course of formalin-induced edema and, right, pie charts showing that edema resolved in 60% of mice over a 2-month period. Inflammation resolution was complete by PFD120. FIG. 1F: Intraplantar formalin produced ongoing/spontaneous pain on PFD14 [time in chamber (s) in the conditioned place preference (CPP) test, PFD14]. FIGS. 1G-1H: Contralateral hyperalgesia in formalin-injected animals that had developed hypersensitivity (i.e., vulnerable) and were given gabapentin [FIG. 1G: (GBP, 50 mg-kg⁻¹, IP)], morphine [FIG. 1H: (Mor, 10 mg-kg⁻¹, SC)] or saline (Veh). Tests were conducted 60 min (FIG. 1G: GBP) or 30 min (FIG. 1H: Mor) and 7 days later. FIG. 1I: Segregation between vulnerable and resilient mice over a 3-week period. Overlaid points are individual animal scores. Results are shown as mean±S.E.M. and were analyzed by a Student's t test (FIG. 1F) or two-way ANOVA (FIG. 1B, FIG. 1E, FIG. 1G, FIG. 1H) followed by Bonferroni's post hoc test, as appropriate.

FIGS. 2A-2G. Chemical end-organ damage produces central sensitization in mouse spinal cord of vulnerable mice. FIG. 2A: Dose-dependent nocifensive responses to intraplantar (hind paw) capsaicin (CPS) injection in naïve mice. FIG. 2B: Effects of capsaicin (0.1 μg) in mice that had received contralateral injections of saline (Veh, open circles) or formalin (1%, closed circles) 14 days earlier. Formalin-injected mice were tested for contralateral hypersensitivity on post-formalin day 14, and were divided into two groups, vulnerable (Vuln, second bar) and resilient (Res, third bar) as described in FIG. 1B. FIG. 2C: Quantification of Fos-like immunoreactive cells in dorsal horn superficial laminae I-II. FIG. 2D: Positive correlation (R²=0.362; P=0.017) between nocifensive response to hind-paw injection of capsaicin (CPS, 0.1 μg) and number of Fos-positive cells in dorsal horn superficial laminae (I-II) in lumbar cord of mice that had previously received formalin in the contralateral hind paw. FIG. 2E: Dose-dependent nocifensive response to forepaw CPS injection in formalin-naïve mice; median effective dose (ED₅₀) was ˜0.1 μg. FIG. 2F: Nocifensive response to contralateral forepaw CPS (0.1 μg) injection in Veh- and Vuln formalin-exposed mice. Results are shown as mean±S.E.M. and were analyzed by Student's t test (FIG. 2F) or one-way ANOVA (FIG. 2A, FIG. 2B, FIG. 2C, FIG. 2E) followed by Dunnett's post hoc test, as appropriate. FIG. 2G: Top, spinal cord image reconstructed from ex vivo diffusion tensor imaging (DTI) data and, bottom, fractional anisotropy in spinal cord of Vuln and Res mice, assessed at PFD14. Data are expressed as ratio Vuln/Veh and Res/Veh. A total of 21 mice were used in this experiment and overlaid points are individual animal scores. Mean±S.E.M. **, P<0.01 by two-tailed Student's t test with Bonferroni's correction between vehicle-injected (Veh) and vulnerable or resilient for each spinal cord segment. C, cervical; T, thoracic; L, lumbar. Overlaid points are individual animal scores.

FIGS. 3A-3G. Formalin injection causes emotional, cognitive, vegetative and microstructural changes in forebrain of vulnerable mice. FIG. 3A: Anxiety-like behavior in vulnerable (Vuln) mice on PFD7 assessed with the elevated plus maze test (EPM). Left, time in open arms (s); center, number of entries in open arms; right, number of entries in closed arms. FIG. 3B: Left, long-term memory [exploration time (s) in the 24-h novel-object recognition test (NOR)] and discriminatory index (right) in Vuln mice on PFD14. FIG. 3C: Body-weight gain in Vuln mice on PFD14. Veh mice, open circles; Vuln mice, closed circles. Results are shown as mean±S.E.M. and were analyzed by Student's t test [FIG. 3A, FIG. 3B (right), FIG. 3C] or two-way ANOVA [FIG. 3B (left)] followed by Bonferroni's multiple comparison. FIGS. 3D-3G: Relative volume changes in forebrain structures of vulnerable [Vuln (FIGS. 3D-3E) and resilient [Res (FIGS. 3F-3G)] formalin-treated mice (PFD14). Mice were tested for contralateral hypersensitivity 2 weeks after formalin injection and were divided into vulnerable (FIGS. 3D-3E) and resilient (FIGS. 3F-3G) groups. 60 min after testing, the animals were euthanized and processed for ex vivo DTI. FIG. 3D, FIG. 3F: regional volume in the right hemisphere; FIG. 3E, FIG. 3G: regional volume in the left hemisphere. FIGS. 3D-3G: Results are expressed as ratio Vuln/Veh (FIGS. 3D-3E) and Res/Veh (FIGS. 3F-3G). A total of 21 mice were used in this experiment. Mean±S.E.M. *, P<0.05; **, P<0.01; ***, P<0.001 by two-tailed Student's t test with Bonferroni's correction between vehicle-injected (Veh) and vulnerable or resilient for each brain region. AC, anterior commissure; BLA, basolateral amygdala; CA2, hippocampal Cornus ammonis 2 subfield DF, dorsal fornix; DHF, dorsal hippocampal fissure; IL, infralimbic prefrontal cortex; LO, lateral olfactory tract; TT, tenia tecta. Overlaid points are individual animal scores.

FIGS. 4A-4L. NAAA controls acute nociception and lasting sensory abnormalities produced by formalin. FIG. 4A: Time-course of the response to formalin (1%) in NAAA-null mice (Naaa^{-/-}, bottom curve) and wild-type littermates (WT, top curve). FIG. 4B: Persistent contralateral hyperalgesia, assessed on post-formalin day (PFD)14, in WT but not Naaa^{-/-} mice challenged with 1% formalin. FIG. 4C: Formalin caused paw edema (Δ thickness, mm; PFD14) in WT but not Naaa^{-/-} mice. Vehicle (Veh), open circles; formalin, closed circles; WT mice, left two bars; Naaa^{-/-} mice, right two bars. FIG. 4D: Time-course of the formalin (1% or 3%) response in Naaa^{-/-} (Form 3%) and WT mice (Form 1%). FIG. 4E: Paw edema (PFD14) in WT and Naaa^{-/-} mice challenged with 1% and 3% formalin, respectively. FIG. 4F: Persistent contralateral hyperalgesia (PFD14) in WT but not Naaa^{-/-} mice injected with 1% and 3% formalin respectively. Open circles, WT plus vehicle; closed circles, WT plus 1% formalin; open triangles, Naaa^{-/-} plus 3% formalin. FIGS. 4G-4I: Administration of ARN16186 [ARN (86); 10 mg-kg⁻¹, IP], ARN19702 [ARN (02); 30 mg-kg⁻¹, IP] prevents nocifensive response (FIG. 4G); persistent contralateral hyperalgesia (PFD14) (FIG. 4H); and paw edema (PFD14) (FIG. 4I). Open circles (first bar from the left), saline plus drug vehicle (Veh-Veh); closed circles (second bar from the left), formalin plus drug vehicle (Form-Veh); closed circles (third bar from the left), formalin plus ARN19702 [Form-ARN (02)]; triangles (fourth bar from the left), formalin plus ARN16186 [Form-ARN (86)]. FIG. 4J: Time-course of the response to 0.1% formalin in mice overexpressing NAAA in CD11b+ cells (Naaa^{CD11b+}, top curve) and WT littermates (bottom curve). FIG. 4K: Persistent contralateral hyperalgesia in Naaa^{CD11b+} exposed to 0.1% formalin (PFD14). FIG. 4L: Paw edema in Naaa^{CD11b+} exposed to formalin (0.1%) (PFD14). Vehicle (Veh), open circles; formalin (Form), closed circles; WT mice, left two bars; Naaa^{CD11b+} mice, right two bars. Results are shown as mean±S.E.M. and were analyzed by one-way ANOVA (FIG. 4H, FIG. 4I) or two-way ANOVA (FIGS. 4A-4G and FIGS. 4J-4L) followed by Dunnett's or Bonferroni's post hoc test.

FIGS. 5A-5M. NAAA inhibition halts the emergence of formalin-induced CPLS. FIG. 5A: Time-course of contralateral hyperalgesia in formalin-injected male mice which received ARN19702 (30 mg-kg⁻¹, IP; closed circles, third bar in each set), ARN16186 (10 mg-kg⁻¹, IP, open triangles, fourth bar in each set) or vehicle (open circles, first bar in each set) once daily from PFD1 to PFD3. FIG. 5B: Effects of post-formalin ARN19702 (30 mg-kg⁻¹, IP) administration (PFD1-3) on spontaneous/ongoing pain response [time in chamber (s) during the conditioned place preference test, PFD14]. FIGS. 5C-5E: Effects of post-formalin ARN19702 administration (PFD1-3) on anxiety-like behavior [FIG. 5C; (right) time in open arms (s) of an elevated plus maze and (left) anxiety index, PFD7]; long-term memory [FIG. 5D; exploration time (s) in the 24-h novel-object recognition test, PFD14]; body-weight gain (FIG. 5E; PFD14); fractional anisotropy in spinal cord (FIG. 5F: PFD14); and volume changes in forebrain (FIG. 5G; PFD14); data in FIG. 5F and FIG. 5G are from vulnerable (Vuln) formalin-injected mice and are expressed as ratio ARN19702-treated/untreated animals. FIG. 5H: Contralateral hyperalgesia in formalin-injected female mice which received ARN19702 (30 mg-kg⁻¹, IP; blue circles) or vehicle (open circles) once daily from PFD1 to PFD3. FIGS. 51-5L: The analgesics gabapentin [FIG. 5I; GBP, 50 mg-kg⁻¹, IP]; morphine [FIG. 5J; MOR, 10 mg-kg⁻¹, subcutaneous]; ketamine [FIG. 5K; KET, 4 mg-kg⁻¹, IP]; and ketoprofen [FIG. 5L; KTP, 100 mg-kg⁻¹, IP]; (administered on PFD1-3) do not prevent contralateral hyperalgesia (PFD14) in WT mice treated with formalin. FIG. 5M: Paw edema (A thickness, mm; PFD14) in male mice treated with formalin, formalin plus ARN19702 or their vehicles. Results are expressed as mean±S.E.M. and were analyzed by one-way ANOVA (FIG. 5B, FIG. 5C, FIG. 5E, FIGS. 5H-5M) or two-way ANOVA (FIG. 5A, FIG. 5D) followed by Dunnett's or Bonferroni's post hoc test, as appropriate. Overlaid points are individual animal scores.

FIGS. 6A-6F. NAAA blockade prevents lasting sensory and cognitive abnormalities produced by chronic constriction injury (CCI). FIG. 6A: Effects of post-CCI ARN19702 (30 mg-kg⁻¹, IP) administration (PFD1-3) on spontaneous/ongoing pain response [time in chamber (s) during the conditioned place preference test, POD14]. FIGS. 6B-6C and FIGS. 6E-6F: Time-course of ipsilateral hyperalgesia (FIG. 6B, FIG. 6E) and allodynia (FIG. 6C, FIG. 6F) after sham surgery (open circles) or sciatic nerve ligation (CCI, closed circles). FIGS. 6B-6C: WT mice subjected to CCI and treated with vehicle (second bar in each set) or ARN19702 (30 mg-kg⁻¹, IP; third bar in each set). FIG. 6D: Long-term memory in WT mice which were subjected to sham or CCI surgery and received either ARN19702 or its vehicle. Left, exploration time (s) in the 24-h novel-object recognition test on post-operative days (POD) 26. Right, discriminatory index. FIGS. 6E-6F: Naaa^{-/-} mice subjected to CCI or sham surgery, without drug treatment. Results are expressed as mean±S.E.M. and were analyzed by one-way ANOVA [FIG. 6D (right)] or two-way ANOVA [FIGS. 6B-6C, FIG. 6D (left), FIGS. 6E-6F] followed by Dunnett's or Bonferroni's post hoc test, as appropriate. Overlaid points are individual animal scores. POD: post-operative day.

FIGS. 7A-7C. FAAH inhibition prevents the emergence of formalin-induced CPLS. Time-course of contralateral hyperalgesia (FIG. 7A) and contralateral allodynia (FIG. 7B) in male mice treated with formalin (Form-Veh, second bar in each set), formalin plus URB597 (Form-URB, 3 mg-kg⁻¹, IP; third bar in each set), or their vehicles (Veh-Veh, open circles, first bar in each set) once daily from PFD3 to PFD5. FIG. 7C: Paw edema (A thickness, mm; PFD 7 and 14). Results are expressed as mean±S.E.M. and were analyzed by two-way ANOVA followed by Bonferroni's post hoc test, as appropriate. Overlaid points are individual animal scores.

FIGS. 8A-8J. A critical period for pain chronification. FIG. 8A: Mice received formalin (1%) or saline and were assigned to 7 groups (n=10 mice each): 5 received one dose of ARN19702 (30 mg-kg⁻¹, IP) or vehicle on post-formalin day (PFD)1 or PFD2 to PFD5; two additional groups received one daily dose of ARN19702 or vehicle on PFD6-7 or PFD8-9. Contralateral hyperalgesia was assessed 2 weeks later. FIG. 8B: Effects of ARN19702 (top set of data) or vehicle (bottom set of data) on CPLS establishment. Open circles, no formalin. FIG. 8C: Time-course of Naaa transcription in ipsilateral (left) or contralateral (right) L4-L6 hemicord of mice treated with vehicle (open circles) or formalin (closed circles). FIGS. 8D-8G: Localization of immunoreactive NAAA (irNAAA) in ipsilateral L4-L6 hemicord of vehicle- (FIG. 8D) and formalin (FIG. 8E)-injected mice (PFD4). NAAA partially colocalized with NeuN. Magnification: 10× (FIGS. 8D-8E) or 40× [FIG. 8E (bottom), zoom 2×]. Scale bar, 20 μm. FIG. 8F: Quantification of irNAAA in ipsilateral dorsal horn (DH), ventral horn (VH) and white matter (WM) in L5 cord of vehicle- or formalin-injected mice. FIG. 8G: Quantification of NAAA-immunoreactive NeuN+ cells in ipsilateral DH and VH in L5 cord of vehicle- or formalin-injected mice. FIG. 8H: PEA levels in ipsilateral L4-L6 hemicord fragments of vehicle- (open circles) or formalin-treated mice (closed circles) (PFD4). FIG. 8I: Effects of intrathecal (IT) ARN19702 or ARN077 (30 ng, 5 μL) at PFD2 and PFD4 on hypersensitivity at PFD14. FIG. 8J: Effects of intrathecal (IT) ARN19702 at post-operative 2 and 4 on hypersensitivity at post-operative day 14. Vehicles, open circles; formalin, closed circles (second bar); NAAA inhibitors, closed circles (third bar). Mean±S.E.M of n=4-10 mice per group. *, P<0.05; ***, P<0.001, Student's t test with Bonferroni's correction (FIG. 8G), one-way ANOVA (FIG. 8B, FIG. 8C, FIG. 8H, FIG. 8I, FIG. 8J) or two-way ANOVA (FIG. 8A) followed by Dunnett's or Bonferroni's post hoc test.

FIGS. 9A-9N. NAAA controls the transition to pain chronicity via PEA signaling at PPAR-α. FIG. 9A: PEA content in ipsilateral lumbar cord (L4-L6) in mice treated with intraplantar saline (Veh, open circles) or formalin (Form, closed circles) and then with ARN19702 (30 mg-kg⁻¹, IP; third bar) or its vehicle (second bar) on post-formalin day (PFD)2-4. Mice were euthanized for lipid analysis 2 h after ARN19702 administration. FIGS. 9B-9D: Effects of intraplantar formalin (0.1%) injection on acute nocifensive behavior (FIG. 9B); paw edema [Δ thickness, mm; PFD14] (FIG. 9C); and contralateral hyperalgesia (withdrawal latency, s; PFD14) in Ppara^{-/-} (triangles) or wild-type (WT) mice (circles) (FIG. 9D). FIGS. 9E-9G: Effects of formalin (1%) on acute nocifensive behavior (FIG. 9E); paw edema (PFD14) (FIG. 9F); and contralateral hyperalgesia (PFD14) in Ppara^{-/-} and WT mice (FIG. 9G). FIGS. 9H-9I: Contralateral hyperalgesia (PFD14) in Ppara^{-/-} mice after administration of ARN19702 (FIG. 9H); and WT mice after administration of ARN19702 plus PPAR-α antagonist GW6471 (4 mg-kg⁻¹, IP, 15 min prior to ARN19702) (FIG. 9I). FIGS. 9J-9L: Contralateral hyperalgesia after administration of PPAR-α agonist GW7647 (10 mg-kg⁻¹, IP) on post-procedural day 2-4 in WT mice exposed to 1% formalin (FIG. 9J); NAAA-overexpressing mice (Naaa^{CD11b+}) exposed to 0.1% formalin (FIG. 9K); and WT mice subjected to sciatic nerve ligation (CCI) (FIG. 9L). FIGS. 9M-9N: Effects of administration of PEA (30 mg-kg⁻¹, IP) (FIG. 9M) or fenofibrate (Feno, 100 mg-kg⁻¹, IP, closed circles) on PFD2-4 (FIG. 9N). Overlaid points are individual animal scores. Mean±S.E.M of n=4-10 mice per group. Ns, non-significant; *, P<0.05; **, P<0.01; ***, P<0.001 by one-way ANOVA (FIG. 9A, FIGS. 9H-9N) or two-way ANOVA (FIGS. 9B-9G) followed by Dunnett's or Bonferroni's post hoc test, as appropriate.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

A “detectable agent” or “detectable moiety” is a substance (e.g., compound) or composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g., a first linker or second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like).

The term “capable of binding” as used herein refers to a moiety (e.g., a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waal's bond(s)/interactions, hydrogen bond(s), polar bond(s), or combinations or mixtures thereof).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

An “inhibitor” refers to a compound (e.g., compounds described herein) that reduces activity when compared to a control, such as absence of the compound or a compound with known inactivity.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., a protein associated disease, a cancer (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

A “therapeutic agent” or “drug agent” as used herein refers to an agent (e.g., compound or composition) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. A drug moiety is a monovalent drug. A therapeutic moiety is a monovalent therapeutic agent.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

As used herein, a “pathological state” refers to inflammatory conditions, neurodegenerative disorders, pain, a corneal neovascularization, diabetic retinopathy, dry macular degeneration, migraine, neuropathy, post herpetic neuralgia, trigeminal neuralgia, causalgia, diabetic neuropathy, chronic pain, nociceptive pain, complex regional pain syndrome (CRPS), neurogenic pain (including, but not limited to neuropathic pain, central pain and deafferentation pain), peripheral or polyneuropathie pain, toxic neuropathy, chronic neuropathy caused by chemotherapeutic and antiviral agents, nociceptive pain, or pruritus induced by uremia, pain associated with cancers, malignancies of various origin, polycythemia, jaundice or cholestasis, iron deficiency, athlete's foot, xerosis, wound healing, thyroid illness, hyperparathyroidism, or menopause, glossopharyngeal neuralgia, occipital neuralgia, pain, postherpetic neuralgia, retinopathy of prematurity, sinus headache, trigeminal neuralgia, or wet macular degeneration. In embodiments, pain, particularly severe pain, can be a stressor. In embodiments, provided herein are methods of treating chronic pain conditions, including neuropathic pain, and chronic or intermittent pain associated with chronic health conditions as such conditions are often substantial stressors.

In embodiments, “neuropathic pain” may include pain caused by a primary lesion or dysfunction of the nervous system. Such pain may be chronic and involve a maintained abnormal state of increased pain sensation, in which a reduction of pain threshold and the like are continued, due to persistent functional abnormalities ensuing from an injury or degeneration of a nerve, plexus or perineural soft tissue. Such injury or degeneration may be caused by wound, compression, infection, cancer, ischemia, or a metabolic or nutritional disorder such as diabetes mellitus. Neuropathic pain may include, but is not limited to, neuropathic allodynia wherein a pain sensation is induced by mechanical, thermal or another stimulus that does not normally provoke pain, neuropathic hyperalgesia wherein an excessive pain occurs in response to a stimulus that is normally less painful than experienced. Examples of neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. In embodiments, the neuropathic pain includes the pain caused by either central or peripheral nerve damage. In embodiments, it includes the pain caused by either mononeuropathy or polyneuropathy (e.g., familial amyloid polyneuropathy). In embodiments, as compared to inflammatory pain, neuropathic pain is resistant to therapy with nonsteroidal anti-inflammatory agents and opioid substances (e.g., morphine). Neuropathic pain may be bilateral in mirror image sites, or may be distributed approximately according to the innervation of the injured nerve, it may persist for months or years, and be experienced as burning, stabbing shooting, throbbing, piercing electric shock, or other unpleasant sensation.

The term “acute pain” is used in accordance with its plain and ordinary meaning and refers to the physiologic response and experience to noxious stimuli. In embodiments, the acute pain is sudden in onset and is time-limited. As used herein, the term “acute pain state” is a state of acute pain.

The term “chronic pain” is used in accordance with its plain and ordinary meaning and refers to pain that is ongoing and lasts longer than three months. In embodiments, the chronic pain lasts longer than six months. As used herein, the term “chronic pain state” is a state of chronic pain.

As used herein, the term “inflammatory condition” refers to a disease or condition characterized by aberrant inflammation (e.g., an increased level of inflammation compared to a control such as a healthy person not suffering from a disease). Examples of inflammatory condition include postoperative cognitive dysfunction, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barré syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. In embodiments, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

As a non-limiting example, the compounds described herein can be co-administered with anti-cancer agents or conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin, etc.), and the like.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer, or with adjunctive agents that may not be effective alone, but may contribute to the efficacy of the active agent.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. In embodiments, an anti-cancer agent is an inhibitor of K-Ras, RAF, MEK, Erk, PI3K, Akt, RTK, or mTOR. In embodiments, an anti-cancer agent is an MDM2 inhibitor or a genotoxic anti-cancer agent. In embodiments, an anti-cancer agent is nutlin-1, nutlin-2, nutlin-3, nutlin-3a, nutlin-3b, YH239-EE, MI-219, MI-773, MI-77301, MI-888, MX69, RG7112, RG7388, RITA, idasanutlin, DS-3032b, or AMG232. In embodiments, an anti-cancer agent is an alkylating agent, intercalating agent, or DNA replication inhibitor. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e., paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, Inanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

“Chemotherapeutic” or “chemotherapeutic agent” is used in accordance with its plain ordinary meaning and refers to a chemical composition or compound having antineoplastic properties or the ability to inhibit the growth or proliferation of cells.

“Anti-diabetic agent” or “antidiabetic agent” is used in accordance with its plain ordinary meaning and refers to a composition (e.g., compound, drug, antagonist, inhibitor, modulator) having the ability to lower blood glucose levels in a subject. In some embodiments, an anti-diabetic agent is an agent identified herein having utility in methods of treating diabetes. In some embodiments, an anti-diabetic agent is an agent approved by the FDA or similar regulatory agency of a country other than the USA, for treating diabetes. Examples of anti-diabetic agents include, but are not limited to, insulin, insulin sensitizers (e.g., biguanides (e.g. metformin, phenformin, or buformin), thiazolidinediones (e.g., rosiglitazone, pioglitazone, troglitazone)), secretagogues (e.g., sulfonylureas (e.g., tolbutamide, acetohexamide, tolazamide, chlorpropamide, glipizide, glyburide, glibenclamide, glimepiride, gliclazide, glycopyramide, gliquidone), meglitinides (e.g., repaglinide, nateglinide)), alpha-glucosidase inhibitors (e.g., miglitol, acarbose, voglibose), peptide analog antidiabetic agents (e.g., incretins (glucagon-like peptide-1, gastric inhibitory peptide), glucagon-like peptide agonists (e.g., exenatide, liraglutide, taspoglutide), gastric inhibitoty peptide analogs, or dipeptidyl peptidase-4 inhibitors (e.g., vildagliptin, sitagliptin, saxagliptin, linagliptin, allogliptin, septagliptin), amylin agonist analogues (e.g., pramlintide).

The terms “N-acylethanolamine acid amidase”, “NAAA”, and “hNAAA” are used according to the plain and ordinary meaning in the art and refer to a 31 kDa enzyme by the same name involved in the hydrolysis of non-peptidic amides. The term “NAAA” may refer to the nucleotide sequence or protein sequence of human NAAA (e.g., Entrez 27163, Uniprot Q02083, RefSeq NM_014435, or RefSeq NP_055250). The term “NAAA” includes both the wild-type form of the nucleotide sequences or proteins as well as any mutants thereof. In some embodiments, “NAAA” is wild-type NAAA receptor. In some embodiments, “NAAA” is one or more mutant forms. The term “NAAA” XYZ refers to a nucleotide sequence or protein of a mutant NAAA wherein the Y numbered amino acid of NAAA that normally has an X amino acid in the wildtype, instead has a Z amino acid in the mutant. In embodiments, an NAAA is the human NAAA. In embodiments, the NAAA has the nucleotide sequence corresponding to reference number GI: 109148549. In embodiments, the NAAA has the nucleotide sequence corresponding to RefSeq NM_014435.3. In embodiments, the NAAA has the protein sequence corresponding to reference number GI:109148550. In embodiments, the NAAA has the protein sequence corresponding to RefSeq NP_055250.2. In embodiments, NAAA functions in acidic conditions (e.g., pH about 4.5-5.0).

The term “FAAH” denotes a mammalian Fatty Acid Amide Hydrolase and includes, but is not limited to, the human, rat, and mouse forms of the enzyme. U.S. Pat. No. 6,271,015 discloses isolated and purified forms of FAAH. Fatty Amide Hydrolases (FAAHs) (Deutsch, D. G., et al., Prostaglandins Leukot. Essent. Fatty Acid, 66, 201-210 (2002)) are enzymes responsible for the degradation of lipid ethanolamides (Fowler, C. J., et al., Biochem. Pharmacol. 62, 517-526 (2001); Patricelli, M. P., et al., Vitam. Horm., 62, 663-674 (2001)), e.g., anandamide (Devane, W. A., et al., Science 258, 1946-1949 (1992)), oleoylethanolamide (Rodríguez de Fonseca, F., et al., Nature (London) 414, 209-212 (2001); Fu, J., et al., Nature (London) 425, 90-93 (2003)), and palmitoylethanolamide (Calignano, A., et al., Nature (London) 394, 277-281 (1998); Lambert, D. M., et al., Curr. Med. Chem. 9, 663-674 (2002)). Owing to the various and important physiological roles of fatty acid ethanolamides, classes of small-molecule compounds able to block FAAH or FAAHs but not bind to other endocannabinoid-metabolizing enzymes, e.g., monoglyceride lipase (MGL) (Dinh, T. P., et al., Proc. Natl. Acad. Sci. U.S.A. 99, 10819-10824 (2002)), or cannabinoid receptors, would be advantageous both as pharmacological tools and as prototypes for drug development projects (Piomelli, D., et al., Trends Pharmacol. Sci. 21, 218-224 (2000); Bisogno, T., et al., Curr. Pharm. Des. 8, 533-547 (2002); Yarnell, A., Chem. Eng. News 80(49), 32 (2002); Smith, A., Nat. Rev. Drug Discov. 2, 92 (2003); Wendeler, M., et al., Angew. Chem. Int. Ed. 42, 2938-2941 (2003)).

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### II. Methods

In an aspect is provided a method of stimulating mitochondrial respiration in a nerve cell, including administering an agent to the nerve cell, wherein the agent is an NAAA (N-Acylethanolamine acid amidase) inhibitor, FAAH (Fatty acid amide hydrolase) inhibitor, PPARα (peroxisome proliferator-activated receptor-α) agonist, PEA (palmitoylethanolamide), acetyl-L-carnitine, α-lipoic acid, or olesoxime. In embodiments, the agent is an NAAA (N-Acylethanolamine acid amidase) inhibitor. In embodiments, the agent is a FAAH (Fatty acid amide hydrolase) inhibitor. In embodiments, the agent is a PPARα (peroxisome proliferator-activated receptor-α) agonist. In embodiments, the agent is PEA (palmitoylethanolamide). In embodiments, the agent is acetyl-L-carnitine. In embodiments, the agent is α-lipoic acid. In embodiments, the agent is olesoxime.

In an aspect is provided a method of treating pain in a subject in need thereof, the method including administering an effective amount of an agent to the subject, wherein the agent is administered between about 1 day and 7 days after a traumatic pain event and wherein the agent is an NAAA (N-Acylethanolamine acid amidase) inhibitor, FAAH (Fatty acid amide hydrolase) inhibitor, PPARα (peroxisome proliferator-activated receptor-α) agonist, PEA (palmitoylethanolamide), acetyl-L-carnitine, α-lipoic acid, or olesoxime. In embodiments, the agent is administered between about 24 hours and about 96 hours after the traumatic pain event. In embodiments, the agent is an NAAA (N-Acylethanolamine acid amidase) inhibitor. In embodiments, the agent is a FAAH (Fatty acid amide hydrolase) inhibitor. In embodiments, the agent is a PPARα (peroxisome proliferator-activated receptor-α) agonist. In embodiments, the agent is PEA (palmitoylethanolamide). In embodiments, the agent is acetyl-L-carnitine. In embodiments, the agent is α-lipoic acid. In embodiments, the agent is olesoxime. In embodiments, the agent is administered in a therapeutically effective amount.

In embodiments, the pain is neuropathic pain, nociceptive pain, chronic pain, neuropathy glossopharyngeal neuralgia, occipital neuralgia, postherpetic neuralgia, trigeminal neuralgia, post herpetic neuralgia, trigeminal neuralgia, causalgia, diabetic neuropathy, complex regional pain syndrome (CRPS), neurogenic pain, peripheral pain, polyneuropathie pain, toxic neuropathy, chronic neuropathy or pruritus.

In embodiments, the agent prevents the transition from an acute pain state to a chronic pain state. In embodiments, the agent prevents chronic pain in a subject with acute pain.

In embodiments, the subject is a cancer patient. In embodiments, the subject is a diabetic patient.

In an aspect is provided a method of preventing chronic pain in a subject with acute pain, the method including administering an effective amount of an agent to the subject, wherein the agent is administered between about 1 day and 7 days after a traumatic pain event and wherein said agent is an NAAA (N-Acylethanolamine acid amidase) inhibitor, FAAH (Fatty acid amide hydrolase) inhibitor, PPARα (peroxisome proliferator-activated receptor-α) agonist, PEA (palmitoylethanolamide), acetyl-L-carnitine, α-lipoic acid, or olesoxime. In embodiments, the method includes preventing transition from an acute pain state to a chronic pain state in the subject. In embodiments, the agent is administered between about 24 hours and about 96 hours after the traumatic pain event. In embodiments, the agent is an NAAA (N-Acylethanolamine acid amidase) inhibitor. In embodiments, the agent is a FAAH (Fatty acid amide hydrolase) inhibitor. In embodiments, the agent is a PPARα (peroxisome proliferator-activated receptor-α) agonist. In embodiments, the agent is PEA (palmitoylethanolamide). In embodiments, the agent is acetyl-L-carnitine. In embodiments, the agent is α-lipoic acid. In embodiments, the agent is olesoxime. In embodiments, the agent is administered in a therapeutically effective amount.

In embodiments, the method further includes administering the agent between about 8 days and 30 days after the traumatic pain event. In embodiments, the method further includes administering the agent between about 8 days and 10 days after the traumatic pain event. In embodiments, the method further includes administering the agent between about 8 days and 14 days after the traumatic pain event. In embodiments, the method further includes administering the agent between about 8 days and 21 days after the traumatic pain event. In embodiments, the method further includes administering the agent between about 8 days and 28 days after the traumatic pain event. In embodiments, the method further includes administering the agent between about 10 days and 14 days after the traumatic pain event. In embodiments, the method further includes administering the agent between about 10 days and 21 days after the traumatic pain event. In embodiments, the method further includes administering the agent between about 10 days and 28 days after the traumatic pain event. In embodiments, the method further includes administering the agent between about 15 days and 20 days after the traumatic pain event. In embodiments, the method further includes administering the agent between about 15 days and 30 days after the traumatic pain event.

In embodiments, the method further includes continually administering the agent between about 8 days and 30 days after the traumatic pain event. In embodiments, the method further includes continually administering the agent between about 8 days and 10 days after the traumatic pain event. In embodiments, the method further includes continually administering the agent between about 8 days and 14 days after the traumatic pain event. In embodiments, the method further includes continually administering the agent between about 8 days and 21 days after the traumatic pain event. In embodiments, the method further includes continually administering the agent between about 8 days and 28 days after the traumatic pain event. In embodiments, the method further includes continually administering the agent between about 10 days and 14 days after the traumatic pain event. In embodiments, the method further includes continually administering the agent between about 10 days and 21 days after the traumatic pain event. In embodiments, the method further includes continually administering the agent between about 10 days and 28 days after the traumatic pain event. In embodiments, the method further includes continually administering the agent between about 15 days and 20 days after the traumatic pain event. In embodiments, the method further includes continually administering the agent between about 15 days and 30 days after the traumatic pain event.

In embodiments, the traumatic pain event is due to accidental physical injury, invasive surgery, or acute illness. In embodiments, the traumatic pain event is due to accidental physical injury. In embodiments, the traumatic pain event is due to invasive surgery. In embodiments, the traumatic pain event is due to acute illness.

In embodiments, the agent is a NAAA inhibitor, FAAH inhibitor, PPARα agonist, or PEA. In embodiments, the agent is a NAAA inhibitor. In embodiments, the agent is a FAAH inhibitor. In embodiments, the agent is a PPARα agonist. In embodiments, the agent is PEA.

In embodiments, the NAAA inhibitor is ARN16186, ARN077, or ARN19702. In embodiments, the NAAA inhibitor is ARN16186. In embodiments, the NAAA inhibitor is ARN077. In embodiments, the NAAA inhibitor is ARN19702. In embodiments, the NAAA inhibitor is described in J Med Chem. 2020 Jul. 23; 63(14): 7475-7490, which is incorporated herein by reference in its entirety and for all purposes. In embodiments, the NAAA inhibitor is described in WO 2013/078430, US 2013/0281490, WO 2009/049238, US 2014/0094508, WO 2014/144836, US 2016/0068482, WO 2017/201103, or US 2019/0177313, which are incorporated herein by reference in their entirety and for all purposes.

In embodiments, the FAAH inhibitor is URB597, URB937, an analog of URB 597, or an analog of URB 937. In embodiments, the FAAH inhibitor is URB597. In embodiments, the FAAH inhibitor is URB937. In embodiments, the FAAH inhibitor is an analog of URB 597. In embodiments, the FAAH inhibitor is an analog of URB 937. In embodiments, the FAAH inhibitor is described in J Med Chem. 2017 Jan. 12; 60(1):4-46, which is incorporated herein by reference in its entirety and for all purposes. In embodiments, the FAAH inhibitor is described in WO 2015/157313, US 2017/0088510, WO 2012/015704, US 2013/0217764, WO 2013/028570, or US 2014/0288170, which are incorporated herein by reference in their entirety and for all purposes.

In embodiments, the PPARα agonist is natural or unnatural PPARα agonist.

In embodiments, the PPARα agonist is GW7647, PEA (palmitoylethanolamide), or OEA (oleoylethanolamide). In embodiments, the PPARα agonist is GW7647. In embodiments, the PPARα agonist is PEA (palmitoylethanolamide). In embodiments, the PPARα agonist is OEA (oleoylethanolamide). In embodiments, the PPARα agonist is described in Expert Opin Investig Drugs. 2017 May; 26(5):593-60, which is incorporated by reference, herein, in its entirety.

In embodiments, the acute physical injury is a concussion, in bone fracture, or an internal injury. In embodiments, the acute physical injury is a concussion. In embodiments, the acute physical injury is a bone fracture. In embodiments, the acute physical injury is an internal injury.

In embodiments, the invasive surgery is cardiac, breast surgery, or orthopedic surgery. In embodiments, the invasive surgery is cardiac surgery. In embodiments, the invasive surgery is breast surgery. In embodiments, the invasive surgery is orthopedic surgery. In embodiments, the invasive surgery is knee arthroplasty, hip replacement surgery, mastectomy, open-heart surgery, hernia repair, thoracotomy, caesarian section, amputation, or open cholecystoctomy. In embodiments, the invasive surgery is knee arthroplasty. In embodiments, the invasive surgery is hip replacement surgery. In embodiments, the invasive surgery is mastectomy. In embodiments, the invasive surgery is open-heart surgery. In embodiments, the invasive surgery is hernia repair. In embodiments, the invasive surgery is thoracotomy. In embodiments, the invasive surgery is caesarian section. In embodiments, the invasive surgery is amputation. In embodiments, the invasive surgery is open cholecystoctomy.

In embodiments, the agent is administered perioperatively. In embodiments, the agent is administered during the period of time before, during, or after surgery. The term “perioperative” or “perioperatively” is used in accordance with its plain ordinary meaning and generally refers to the time period approximately prior to, during, and/or approximately after a surgical operation. For example, “perioperative” or “perioperatively” may refer to the period of time extending from patient arrival (e.g., admission) at a surgical facility (e.g., hospital, clinic, doctor's office) for a surgical operation until the time the patient leaves (e.g., is discharged from) the surgical operation. In embodiments, perioperative includes surgical facility admission, anesthesia, surgery, and recovery from the surgical operation.

In an aspect is provided a method of preventing chronic pain in a subject, the method including administering perioperatively an effective amount of an agent to the subject, wherein the agent is a NAAA inhibitor, FAAH inhibitor, PPARα agonist, PEA, acetyl-L-carnitine, α-lipoic acid, or olesoxime. In embodiments, the NAAA inhibitor is as described herein, including in embodiments. In embodiments, the FAAH inhibitor is as described herein, including in embodiments. In embodiments, the PPARα agonist is as described herein, including in embodiments.

In an aspect is provided a method of preventing chronic pain in a cancer patient, the method including administering an effective amount of an agent to the cancer patient, wherein said agent is a NAAA inhibitor, FAAH inhibitor, PPARα agonist, PEA, acetyl-L-carnitine, α-lipoic acid, or olesoxime. In embodiments, the NAAA inhibitor is as described herein, including in embodiments. In embodiments, the FAAH inhibitor is as described herein, including in embodiments. In embodiments, the PPARα agonist is as described herein, including in embodiments.

In embodiments, the method further includes administering an anti-cancer agent (e.g., as described herein). In embodiments, the chronic pain is caused by the anti-cancer agent. In embodiments, the chronic pain is peripheral neuropathy. In embodiments, the chronic pain is allodynia. In embodiments, the chronic pain is hyperalgesia. In embodiments, the chronic pain is paresthesia. The term “paresthesia” is used in accordance with its plain ordinary meaning and generally refers to an abnormal sensation, e.g., tingling or pricking. In embodiments, the chronic pain is numbing.

In an aspect is provided a method of preventing chronic pain in a diabetic patient, the method including administering an effective amount of an agent to the diabetic patient, wherein said agent is a NAAA inhibitor, FAAH inhibitor, PPARα agonist, PEA, acetyl-L-carnitine, α-lipoic acid, or olesoxime. In embodiments, the NAAA inhibitor is as described herein, including in embodiments. In embodiments, the FAAH inhibitor is as described herein, including in embodiments. In embodiments, the PPARα agonist is as described herein, including in embodiments.

In embodiments, the chronic pain is chronic peripheral neuropathy. In embodiments, the chronic pain is chronic polyneuropathy. In embodiments, the chronic pain is hyperalgesia. In embodiments, the chronic pain is paresthesia (e.g., tingling or pricking). In embodiments, the chronic pain is numbing.

In embodiments, the agent is not morphine. In embodiments, the agent is not gabapentin. In embodiments, the agent is not ketamine. In embodiments, the agent is not ketoprofen.

### III. Embodiments

Embodiment P1. A method of stimulating mitochondrial respiration in a nerve cell, comprising administering an agent to said nerve cell, wherein the agent is an NAAA (N-Acylethanolamine acid amidase) inhibitor, FAAH (Fatty acid amide hydrolase) inhibitor, PPARα (peroxisome proliferator-activated receptor-α) agonist, PEA (palmitoylethanolamide), acetyl-L-carnitine, α-lipoic acid, or olesoxime.
Embodiment P2. A method of treating pain in a subject in need thereof, the method comprising administering an effective amount of an agent to said patient, wherein said agent is administered between about 1 day and 7 days after a traumatic pain event and wherein said agent is an NAAA (N-Acylethanolamine acid amidase) inhibitor, FAAH (Fatty acid amide hydrolase) inhibitor, PPARα (peroxisome proliferator-activated receptor-α) agonist, PEA (palmitoylethanolamide), acetyl-L-carnitine, α-lipoic acid, or olesoxime.
Embodiment P3. The method of embodiment P2, wherein the traumatic pain event is due to accidental physical injury, invasive surgery, or acute illness.
Embodiment P4. The method of any one of embodiments PI to P3, wherein the agent prevents the transition from an acute pain state to a chronic pain state.
Embodiment P5. The method of embodiment P2, wherein the NAAA inhibitor is ARN16186, ARN077, or ARN19702.
Embodiment P6. The method of embodiment P2, wherein the FAAH inhibitor is URB597, URB937, an analog of URB 597, or an analog of URB 937.
Embodiment P7. The method of embodiment P2, wherein the PPARα agonist is natural or unnatural PPARα agonist.
Embodiment P8. The method of embodiment P2, wherein the PPARα agonist is GW7647, PEA (palmitoylethanolamide), or OEA (oleoylethanolamide).
Embodiment P9. The method of embodiment P3, wherein the acute physical injury is a concussion, in bone fracture, or an internal injury.
Embodiment P10. The method of embodiment P3, wherein the invasive surgery is knee arthroplasty, hip replacement surgery, mastectomy, open-heart surgery, hernia repair, thoracotomy, caesarian section, amputation, or open cholecystoctomy.

### IV. Additional Embodiments

Embodiment 1. A method of preventing chronic pain in a subject with acute pain, the method comprising administering an effective amount of an agent to the subject, wherein said agent is administered between about 1 day and 7 days after a traumatic pain event and wherein said agent is a NAAA inhibitor, FAAH inhibitor, PPARα agonist, PEA, acetyl-L-carnitine, α-lipoic acid, or olesoxime.
Embodiment 2. The method of embodiment 1, further comprising administering said agent between about 8 days and 30 days after the traumatic pain event.
Embodiment 3. The method of embodiment 1, further comprising continually administering said agent between about 8 days and 30 days after the traumatic pain event.
Embodiment 4. The method of one of embodiments 1 to 3, wherein the traumatic pain event is due to physical injury, invasive surgery, or acute illness.
Embodiment 5. The method of embodiment 4, wherein the physical injury is accidental physical injury.
Embodiment 6. The method of embodiment 4, wherein the physical injury is acute physical injury.
Embodiment 7. The method of embodiment 6, wherein the acute physical injury is a concussion, a bone fracture, or an internal injury.
Embodiment 8. The method of embodiment 4, wherein the invasive surgery is knee arthroplasty, hip replacement surgery, mastectomy, open-heart surgery, hernia repair, thoracotomy, caesarian section, amputation, or open cholecystoctomy.
Embodiment 9. The method of embodiment 8, wherein the agent is administered perioperatively.
Embodiment 10. A method of preventing chronic pain in a subject, the method comprising administering perioperatively an effective amount of an agent to the subject, wherein said agent is a NAAA inhibitor, FAAH inhibitor, PPARα agonist, PEA, acetyl-L-carnitine, α-lipoic acid, or olesoxime.
Embodiment 11. A method of preventing chronic pain in a cancer patient, the method comprising administering an effective amount of an agent to the cancer patient, wherein said agent is a NAAA inhibitor, FAAH inhibitor, PPARα agonist, PEA, acetyl-L-carnitine, α-lipoic acid, or olesoxime.
Embodiment 12. The method of embodiment 11, further comprising administering an anti-cancer agent.
Embodiment 13. The method of embodiment 12, wherein the chronic pain is caused by the anti-cancer agent.
Embodiment 14. The method of one of embodiments 11 to 13, wherein the chronic pain is chronic peripheral neuropathy.
Embodiment 15. The method of one of embodiments 11 to 13, wherein the chronic pain is allodynia.
Embodiment 16. A method of preventing chronic pain in a diabetic patient, the method comprising administering an effective amount of an agent to the diabetes patient, wherein said agent is a NAAA inhibitor, FAAH inhibitor, PPARα agonist, PEA, acetyl-L-carnitine, α-lipoic acid, or olesoxime.
Embodiment 17. The method of embodiment 16, wherein the chronic pain is chronic peripheral neuropathy.
Embodiment 18. The method of embodiment 17, wherein the chronic peripheral neuropathy is chronic polyneuropathy.
Embodiment 19. A method of treating pain in a subject in need thereof, the method comprising administering an effective amount of an agent to the subject, wherein said agent is administered between about 1 day and 7 days after a traumatic pain event and wherein said agent is a NAAA inhibitor, FAAH inhibitor, PPARα agonist, PEA, acetyl-L-carnitine, α-lipoic acid, or olesoxime.
Embodiment 20. The method of embodiment 19, further comprising administering said agent between about 8 days and 30 days after the traumatic pain event.
Embodiment 21. The method of embodiment 19, further comprising continually administering said agent between about 8 days and 30 days after the traumatic pain event.
Embodiment 22. The method of one of embodiments 19 to 21, wherein the traumatic pain event is due to physical injury, invasive surgery, or acute illness.
Embodiment 23. The method of embodiment 22, wherein the physical injury is accidental physical injury.
Embodiment 24. The method of embodiment 22, wherein the physical injury is acute physical injury.
Embodiment 25. The method of embodiment 24, wherein the acute physical injury is a concussion, a bone fracture, or an internal injury.
Embodiment 26. The method of embodiment 22, wherein the invasive surgery is knee arthroplasty, hip replacement surgery, mastectomy, open-heart surgery, hernia repair, thoracotomy, caesarian section, amputation, or open cholecystoctomy.
Embodiment 27. The method of embodiment 26, wherein the agent is administered perioperatively.
Embodiment 28. The method of one of embodiments 19 to 27, wherein the subject is a cancer patient.
Embodiment 29 The method of one of embodiments 19 to 27, wherein the subject is a diabetic patient.
Embodiment 30. The method of one of embodiments 1 to 29, wherein the agent is a NAAA inhibitor, FAAH inhibitor, PPARα agonist, or PEA.
Embodiment 31. The method of one of embodiments 1 to 29, wherein the NAAA inhibitor is ARN16186, ARN077, or ARN19702.
Embodiment 32. The method of one of embodiments 1 to 29, wherein the FAAH inhibitor is URB597 or an analog of URB 597.
Embodiment 33. The method of one of embodiments 1 to 29, wherein the FAAH inhibitor is URB937 or an analog of URB 937.
Embodiment 34. The method of one of embodiments 1 to 29, wherein the PPARα agonist is a natural PPARα agonist.
Embodiment 35. The method of one of embodiments 1 to 29, wherein the PPARα agonist is an unnatural PPARα agonist.
Embodiment 36. The method of one of embodiments 1 to 29, wherein the PPARα agonist is GW7647, PEA, or OEA.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

More than 1.5 billion people worldwide suffer from chronic pain, which often starts after an acute pain episode. The sequence of molecular events that lead to pain chronicity is still largely unknown but filling this gap is necessary to identify control nodes that might be targeted by disease-modifying therapies. Here we show, inter alia, that the intracellular cysteine hydrolase N-acylethanolamine acid amidase (NAAA) may constitute one such node. NAAA catalyzes the degradation of palmitoylethanolamide (PEA), an endogenous lipid agonist of the ligand-operated transcription factor peroxisome proliferator-activated receptor-α (PPAR-α). We found that disabling NAAA or enhancing PPAR-α activity in spinal cord during a 72-h time window following peripheral organ damage halts chronic pain development in male and female mice. This effect cannot be ascribed to acute antinociception or antiinflammation and is not mimicked by first-line analgesics morphine, gabapentin, ketamine or ketoprofen. Furthermore, transcriptomic and metabolomic studies revealed that the critical period for NAAA-inhibitor efficacy coincides with a transient rise in NAAA expression in spinal cord segments receiving direct input from the lesioned organ, which suppresses PEA-mediated PPAR-α signaling and reprograms local metabolism from mitochondrial respiration to glycolysis—a Warburg-like effect that steers neuronal energy resources toward the production of biomass for synaptic reorganization. Post-injury NAAA blockade normalizes PEA signaling and stops this metabolic switch along with the emergence of persistent pain. The results identify NAAA as a critical checkpoint for pain chronification and as the first mechanism-based target for disease-modifying agents.

Acute pain after physical trauma can evolve, in vulnerable individuals, into a chronic pain state that long outlasts tissue healing and is often resistant to therapy^{1,2}. Nerve damage is likely to drive this transition but the molecular events underlying it are still poorly understood^{3,4}. Yet delineating the nature, localization and timing of such events is necessary to uncover control nodes in the pain chronification process which could be targeted by disease-modifying medicines⁵. Current research efforts are focused on synaptic³ and innate-immune⁴ adaptations that occur, both peripherally and centrally, following end-organ damage though deficits in neuronal energy balance are also receiving serious consideration^{6,7}.

The intracellular cysteine hydrolase NAAA^{8,9} catalyzes the degradation of PEA, an endogenous lipid agonist of the nuclear receptor PPAR-α¹⁰. This ligand-operated transcription factor serves important regulatory functions in both cellular metabolism and the defensive response to noxious stimuli¹¹. Consistent with this dual role, activation of PPAR-α by exogenous PEA attenuates pain^{10,12} and inflammation¹³ and stimulates mitochondrial respiration¹⁴. These findings prompted us to ask whether NAAA-regulated PEA signaling at PPAR-α might contribute to the emergence of chronic pain after somatic injury, when the pressure to bring about adaptive neuroplastic changes intensifies the demand for bioenergy in peripheral and central nociceptive neurons^{3,15}. The results show that end-organ damage transiently enhances NAAA expression and suppresses PEA-mediated PPAR-α signaling in innervating segments of the spinal cord and, by doing so, redirects local metabolism from high energy-yielding mitochondrial respiration toward biomass-generating aerobic glycolysis, a phenomenon akin to the Warburg effect first described in proliferating cancer cells¹⁶. Disabling NAAA or stimulating PPAR-α during this critical time window stops metabolic reprogramming and aborts the transition to pain chronicity. This unprecedented action identifies NAAA and its cognate signaling complex as the first molecular target for chronic pain modification.

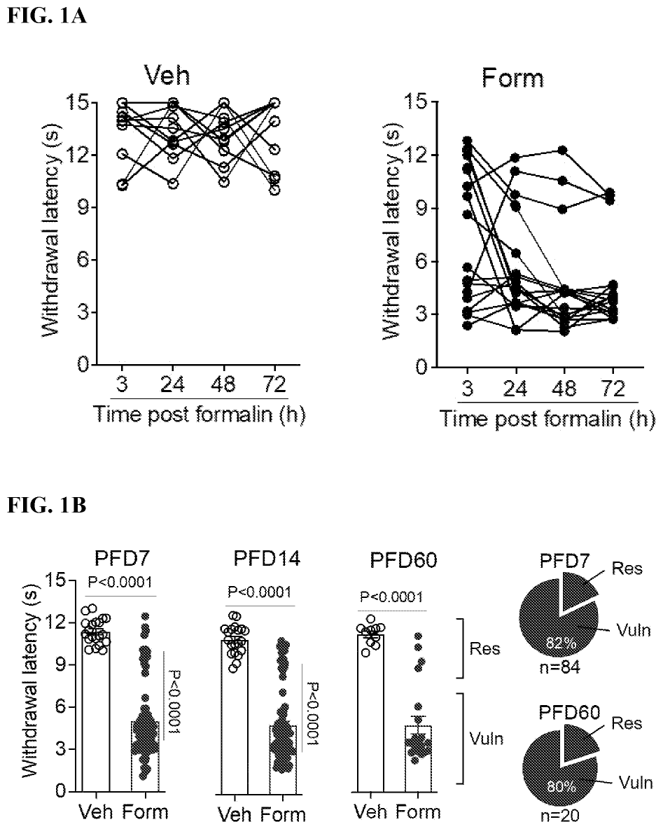

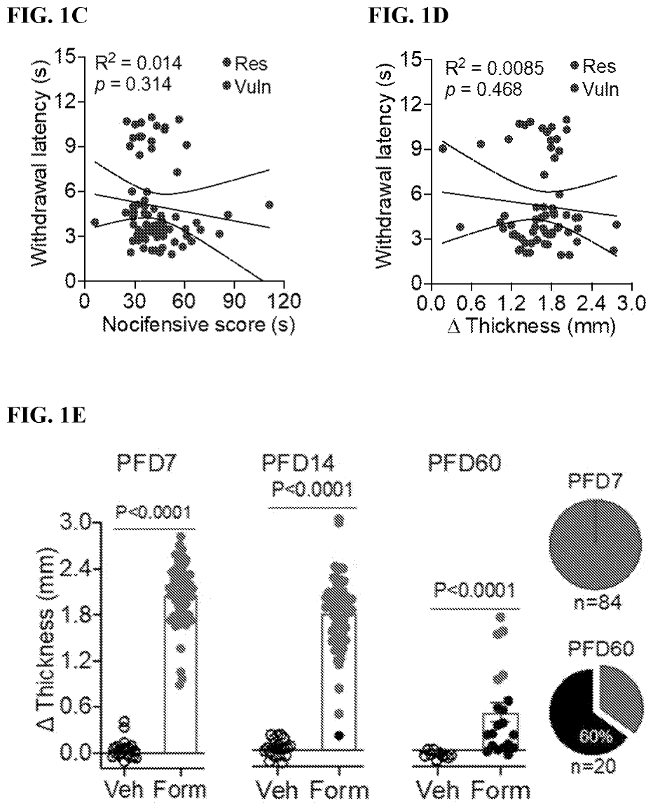

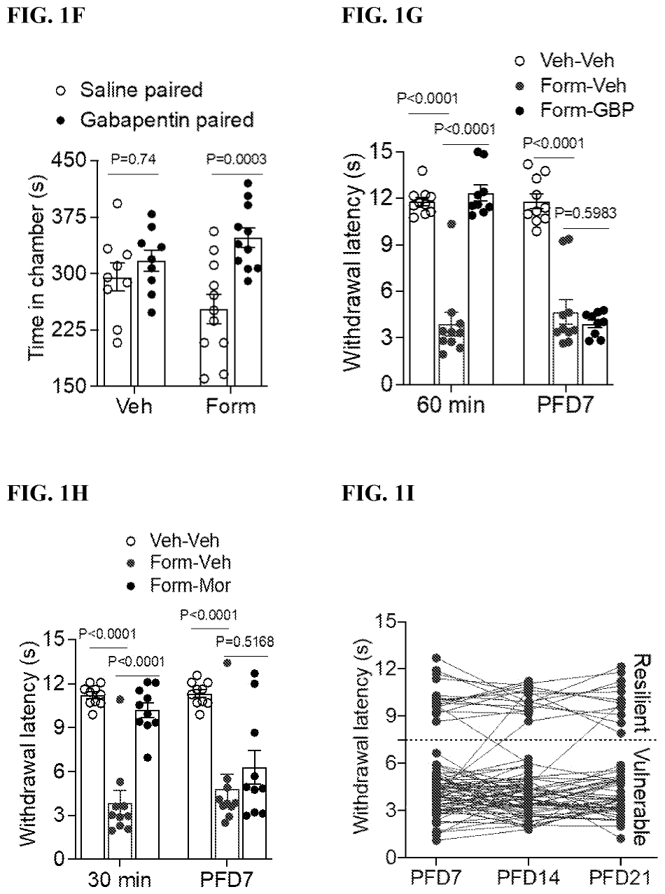

Chemical end-organ damage causes a chronic pain-like state in vulnerable mice: To study pain chronification in a setting that would allow us to measure both acute and persistent pain-related behaviors, we injected formalin (0.1, 0.3 or 1% vol) into the hind paw of male mice and monitored them for the subsequent four months. As expected from prior work¹⁷, formalin evoked an immediate nocifensive reaction that at the 1% concentration lasted >60 min and was associated with local inflammation. Spontaneous pain behavior elicited by 1% (but not 0.1 or 0.3%) formalin, was followed in ˜80% of the animals (67/84) by lasting bilateral hypersensitivity to mildly noxious heat stimuli and normally innocuous mechanical stimuli, which appeared within 24 h (FIG. 1A) and continued unabated until monitoring was ended (FIG. 1B). We obtained similar results in a group of 22 female mice, which all developed hypersensitivity. This state (i) emerged independently of the intensity of the initial nocifensive response or the ensuing inflammatory reaction, which were comparable across all animals (FIG. 1C); (ii) outlasted the resolution of inflammation, which was advanced by post-formalin day (PFD) 60 (FIG. 1E) and ostensibly complete by PFD120; and (iii) was only temporarily alleviated by the analgesics gabapentin and morphine. Finally, there was strict segregation between mice that developed sensory abnormalities and those that did not, with minimal exchange occurring between the two groups over a 3-week period.

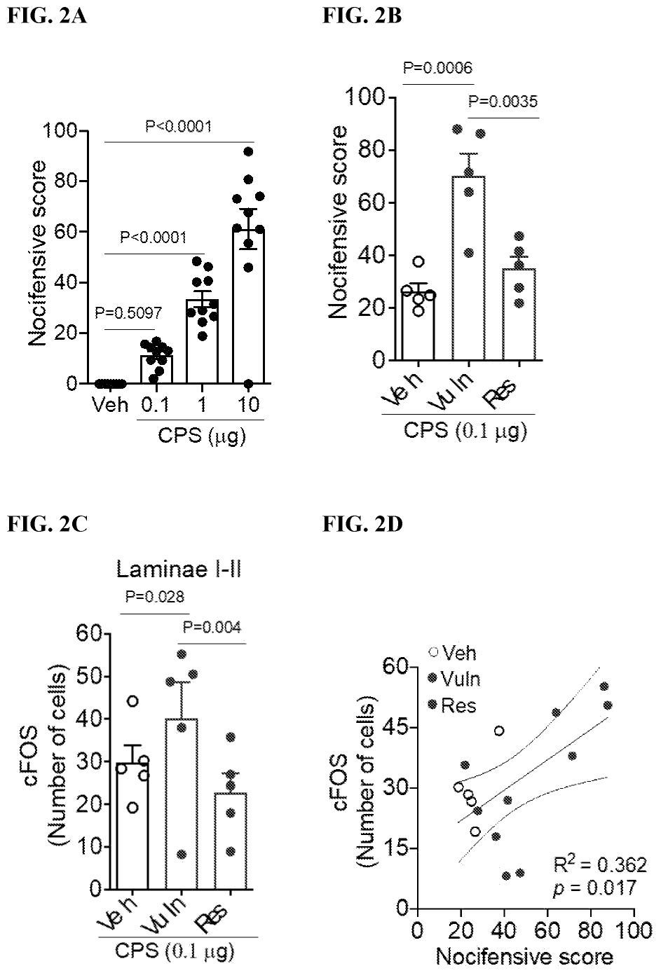

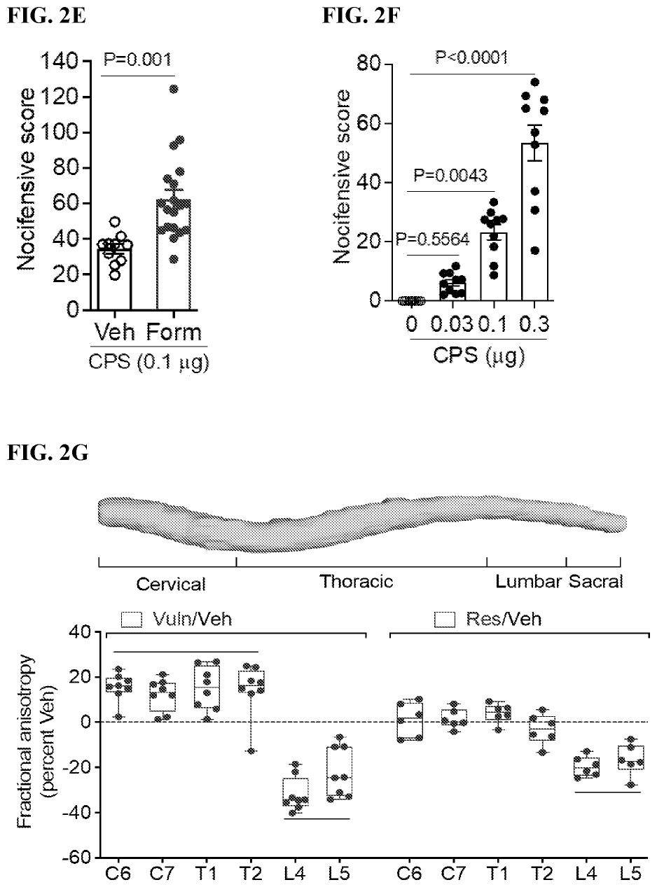

Confirming the emergence of central sensitization¹⁵, we found that administration of a half-maximal dose of the transient receptor potential cation channel subfamily V member 1 (TRPV1) agonist capsaicin (0.1 μg) into the uninjured (contralateral) hind paw evoked stronger nocifensive behavior and bilateral spinal-cord Fos protein expression in animals vulnerable to the lasting effects of formalin, compared to those that were either resilient to such effects or had received only vehicle (FIGS. 2B-2D). Furthermore, two sets of results were suggestive of extraterritorial spreading of sensitization—a common occurrence in chronic pain patients¹⁸. First, forepaw capsaicin injections produced heightened nociception in mice that were previously given formalin in the contralateral hind paw (FIGS. 2E-2F). Second, ex vivo diffusion tensor imaging (DTI) experiments revealed substantial alterations in directional water diffusivity—an index of microstructural integrity¹⁹ and plasticity²⁰—throughout the spinal cord of formalin-exposed mice. Most noteworthy, all animals treated with the irritant exhibited lower fractional anisotropy in the lumbar spinal cord but only those that became hypersensitive displayed higher fractional anisotropy in cord segments innervating the forelimbs (FIG. 2G).

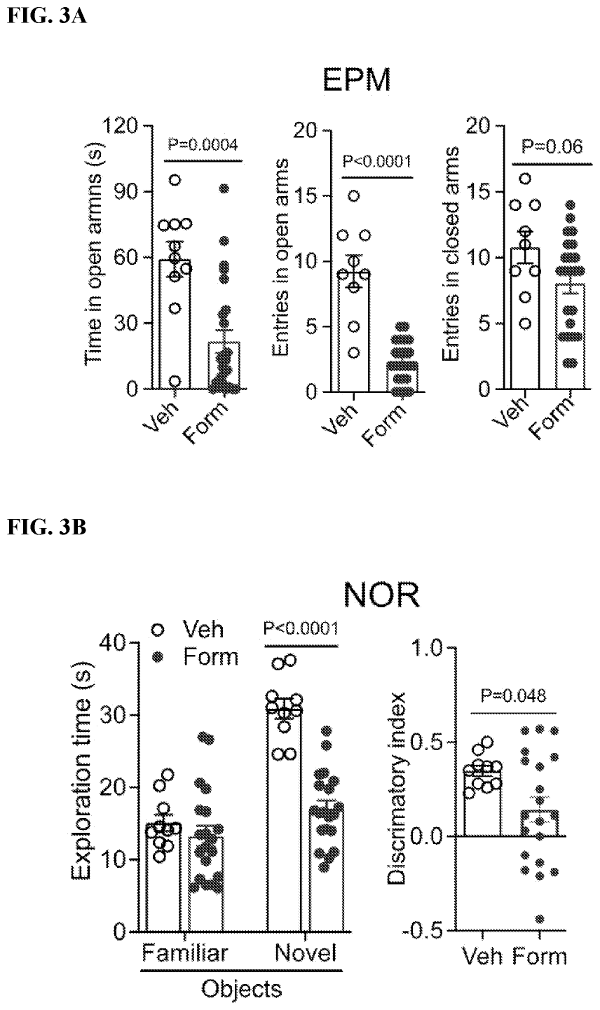

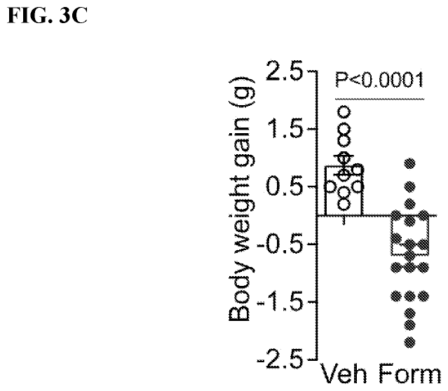

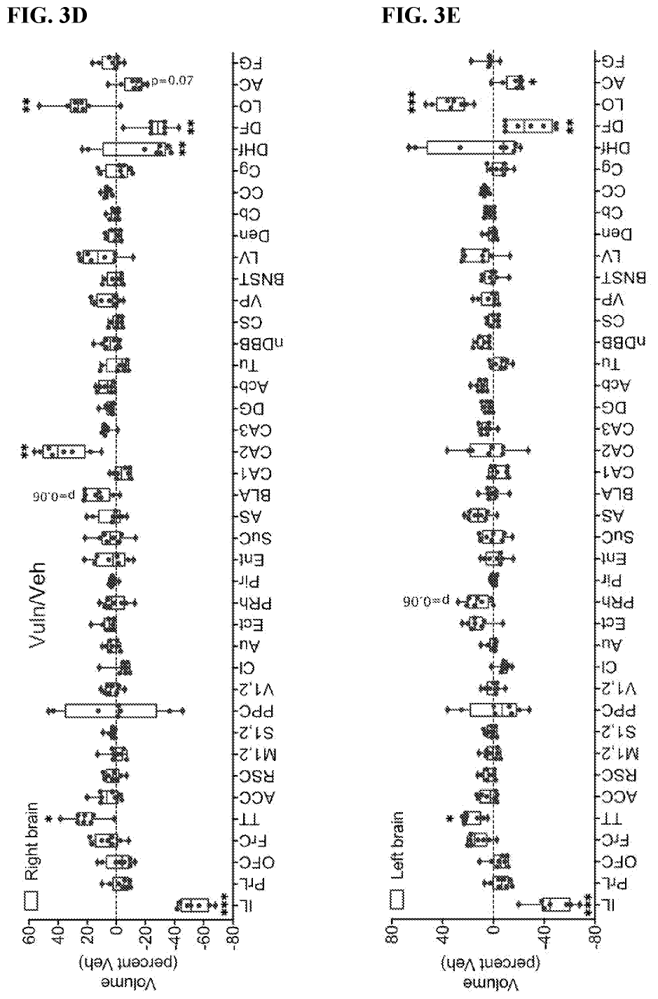

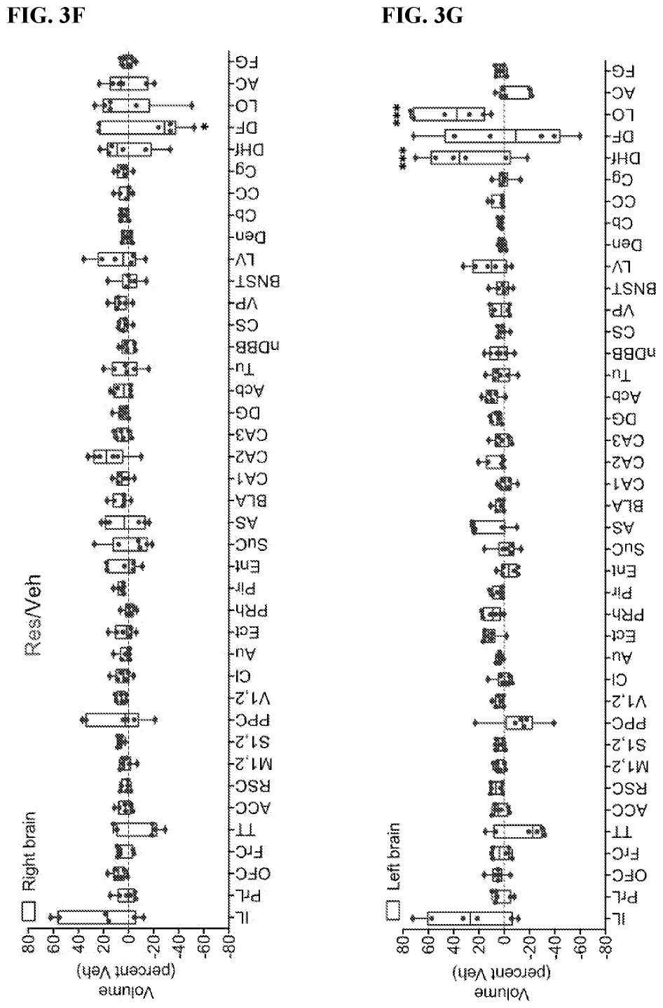

In persons living with chronic pain, sensory abnormalities are often associated with emotional, cognitive and vegetative disturbances as well as with morphological reorganization of the limbic forebrain²¹. Similarly, in mice vulnerable to the enduring effects of formalin, hypersensitivity was accompanied by heightened anxiety-like behavior, defective long-term memory and flattened body-gain trajectory (FIG. 3A (left), FIG. 3B (left), FIG. 3C). There were also conspicuous alterations in forebrain microstructure (FIGS. 3D-3G). Right-hemisphere volume differed between control and vulnerable formalin-exposed mice in three fiber tracts—anterior commissure, dorsal fornix and dorsal hippocampal fissure. Similar white-matter changes were seen in the left hemisphere (FIGS. 3D-3G). Bilateral differences were also observed in the infralimbic prefrontal cortex and tenia tecta, two grey matter regions that participate in the control of pain²², memory²³ and stress^{24,25}. Moreover, unilateral modifications were detected in the right hippocampal CA2 subfield, which is involved in social memory²⁶, and the right basolateral amygdala (P<0.06), whose role in the emotional dimension of pain is well recognized²⁷. Further supporting the functional significance of these alterations, we found only minor microstructural differences between control and resilient formalin-injected animals. The results show that chemical end-organ damage triggers in susceptible mice the development of a long-lasting neuropathological state whose multimodal manifestations are strikingly reminiscent of human chronic pain. For brevity, we will refer to this condition as ‘chronic pain-like state’ (CPLS).

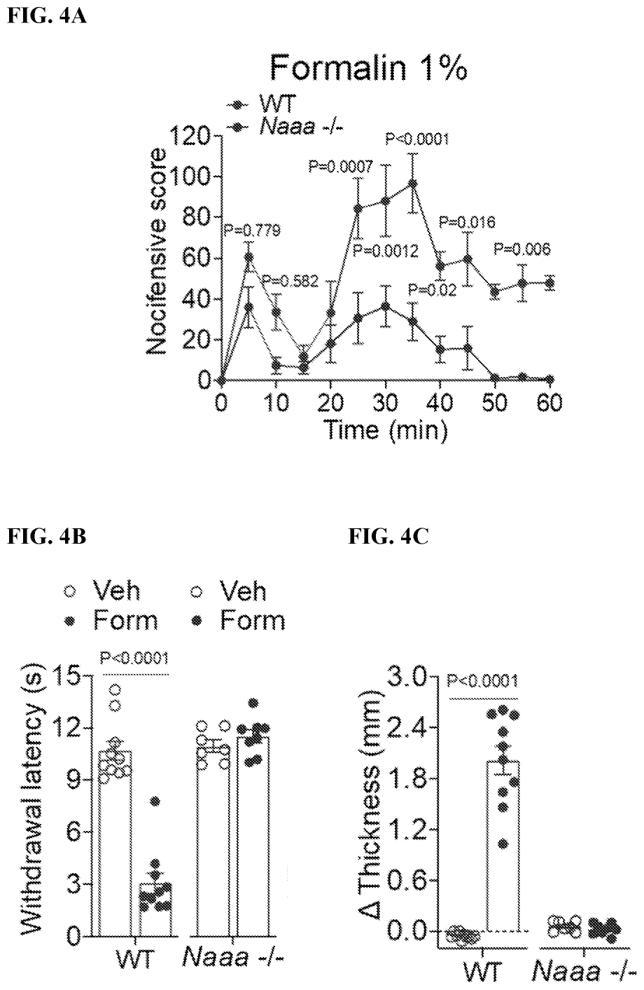

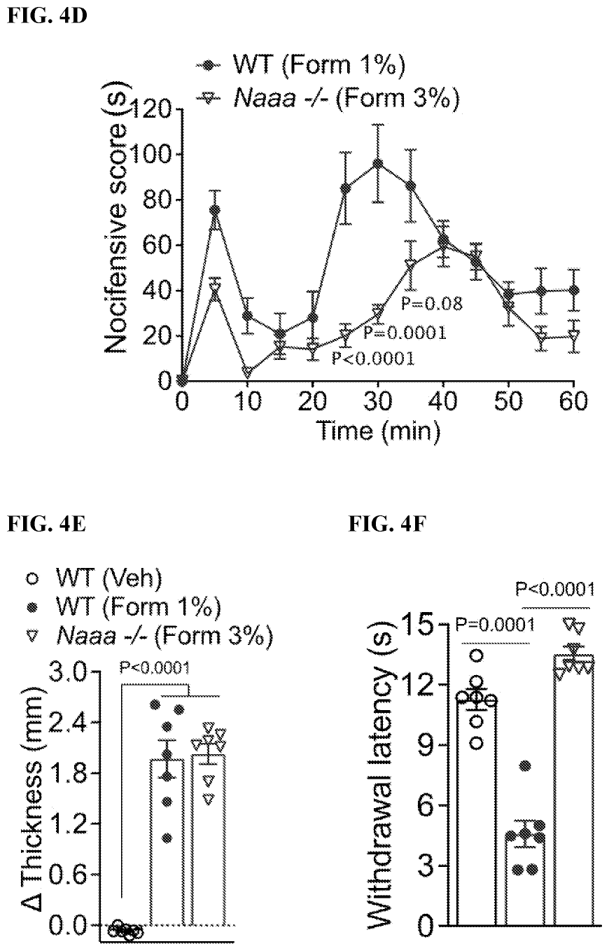

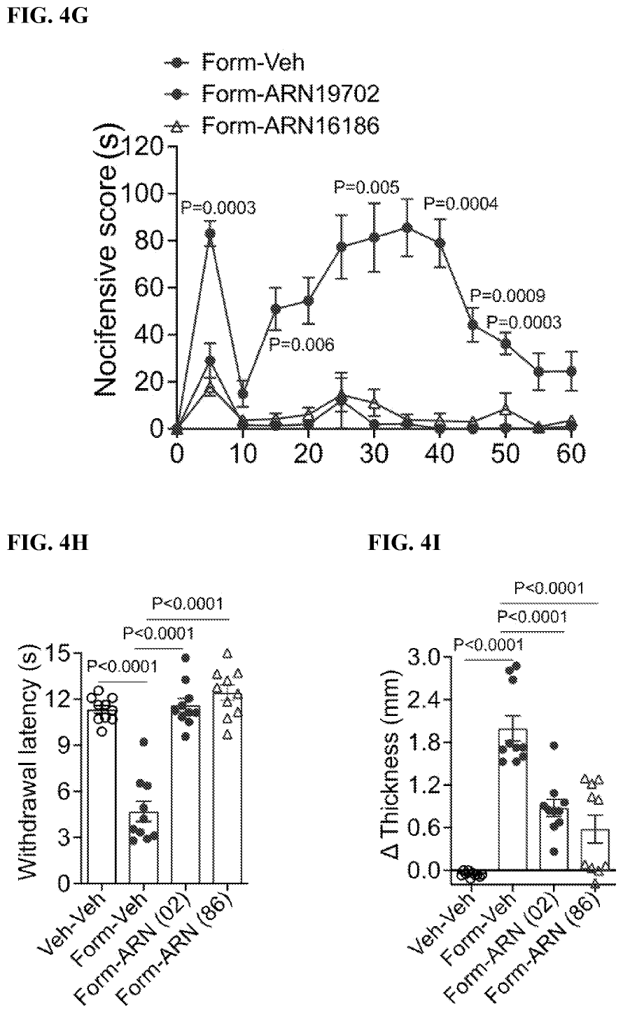

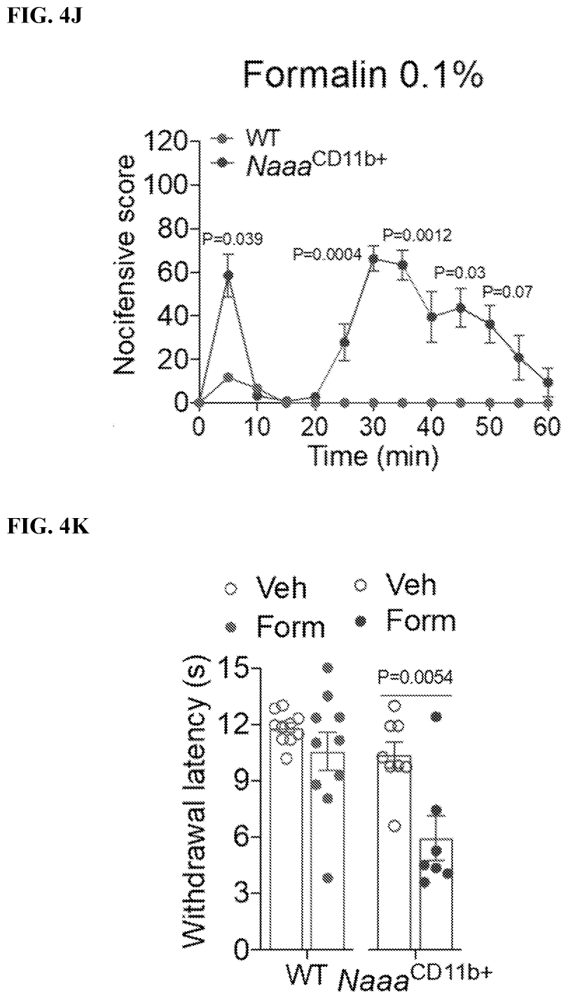

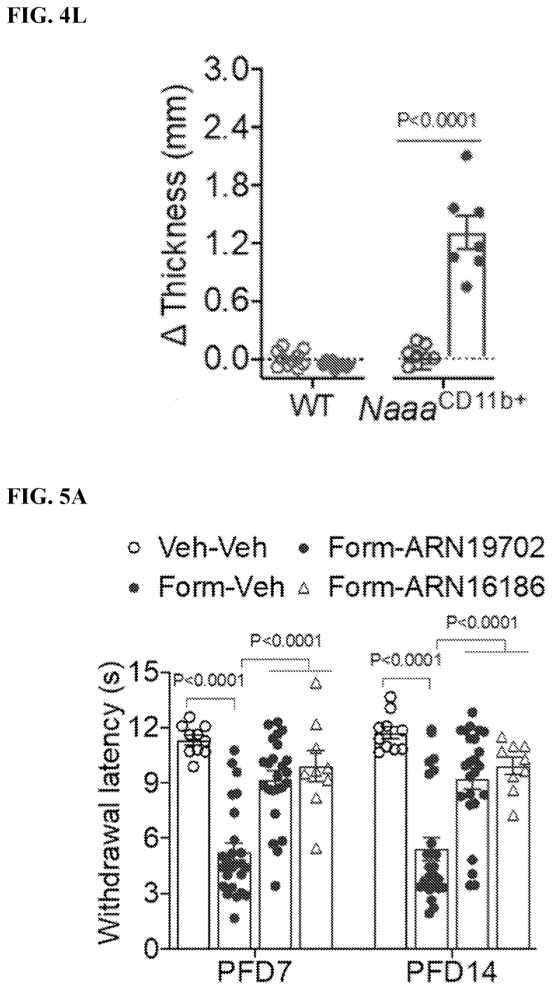

NAAA controls the transition to pain chronicity: We used a genetic strategy as a first step toward assessing whether NAAA might contribute to the emergence of CPLS. Mice constitutively lacking the enzyme²⁸ displayed normal nociceptive thresholds, motor activity and feeding patterns. Nevertheless, formalin (1%) elicited in these mutants a weakened and shorter spontaneous pain response, compared to their wild-type littermates, and did not cause inflammation or persistent hypersensitivity (FIGS. 4A-4C). This phenotype was recapitulated in wild-type mice by pretreatment with either of two reversible and chemically different NAAA inhibitors, ARN19702 and ARN161869. Raising the formalin concentration to 3% increased nociceptive responding and evoked pronounced edema and ipsilateral hypersensitivity in NAAA-null mice but still failed to produce lasting contralateral sensitization. Conversely, mice overexpressing NAAA in monocyte-derived cells, which play important roles in pain chronification^{3,4}, displayed robust nociception, inflammation and persistent hypersensitivity when challenged with a formalin dose (0.1%) that had negligible impact on control animals (FIGS. 4J-4L). The findings suggest that NAAA facilitates acute defensive reactions to injury and point to a possible role for the enzyme in CPLS development.

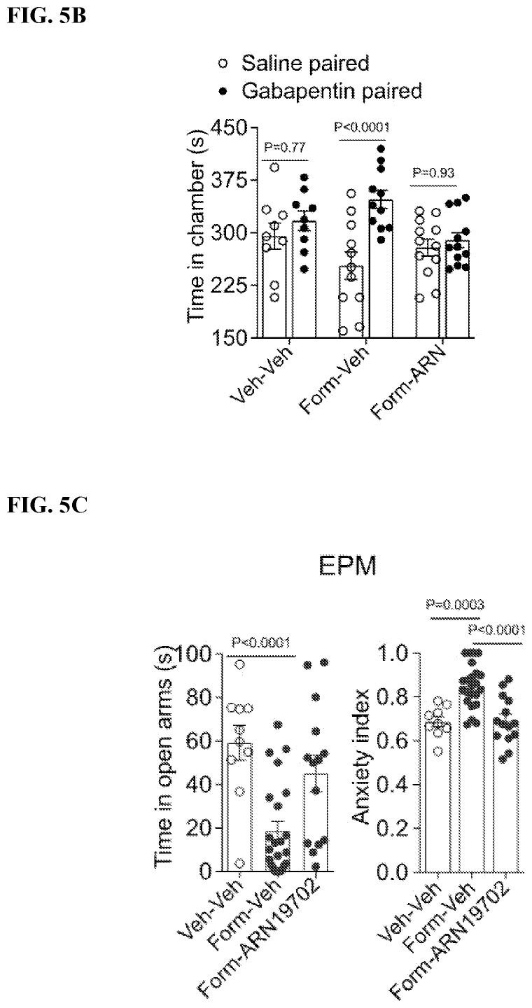

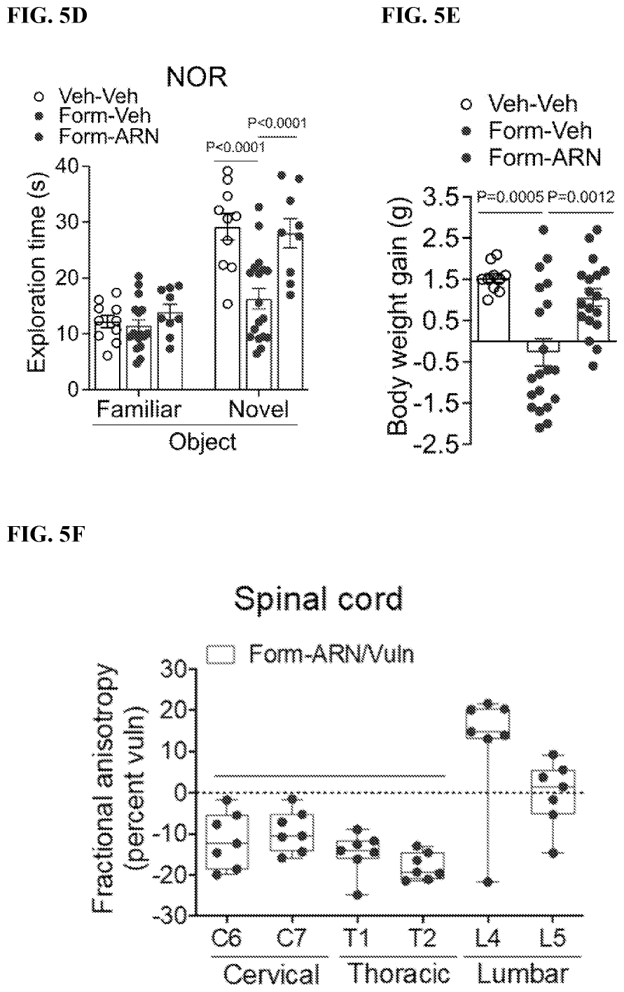

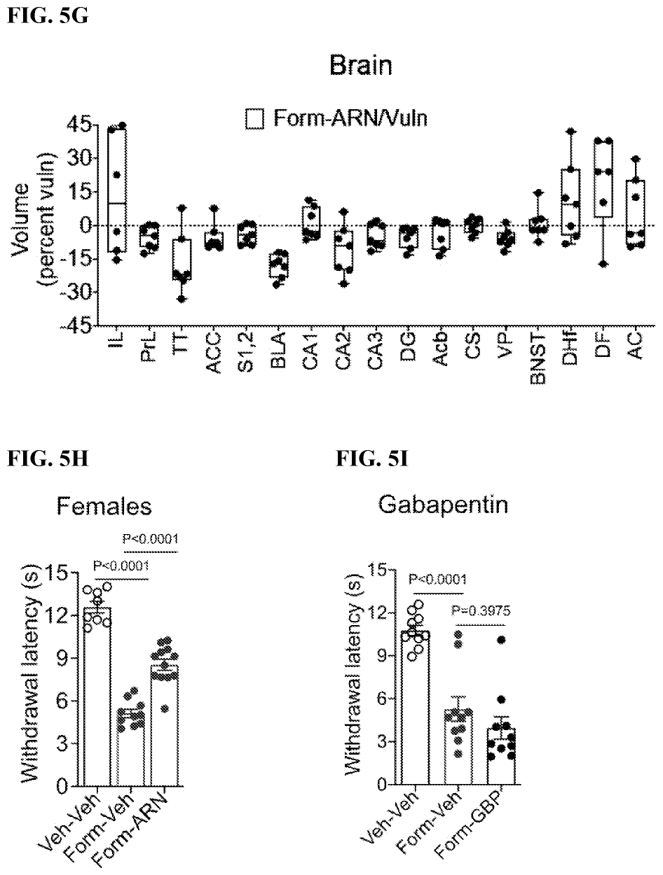

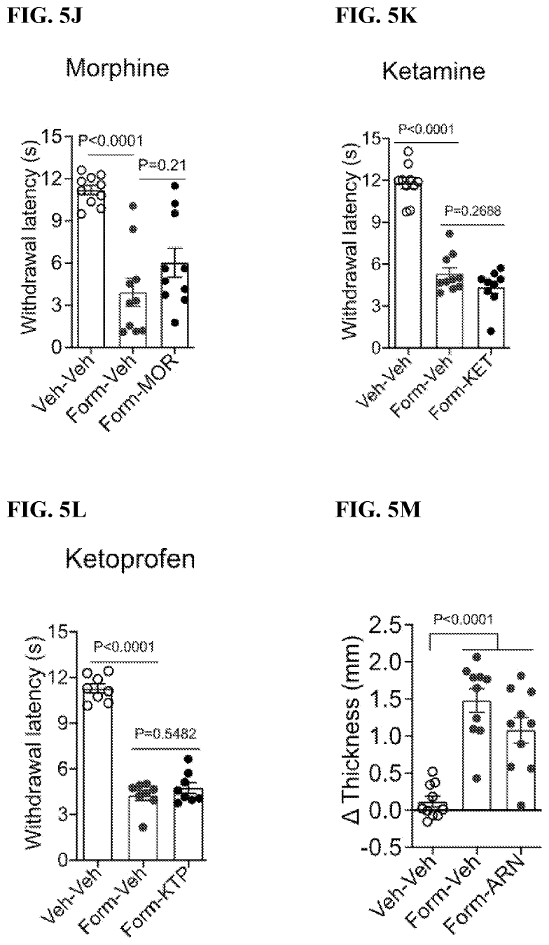

To delineate such role, we administered the NAAA inhibitor ARN19702 (FIG. 5A) or its vehicle to male mice once daily for 3 consecutive days starting 24 h after the formalin challenge, and evaluated sensory, cognitive, emotional, vegetative and microstructural outcomes during the following two weeks. Despite its short duration, the treatment effectively halted CPLS consolidation, normalizing sensory responses, anxiety-like behavior, memory deficits and body-weight gain and reversing CPLS-associated changes in spinal and forebrain DTI measures (FIGS. 5F-5G). Furthermore, the protective effects of ARN19702 were mimicked by ARN16186 and were not sex-dependent.

Unlike agents that interfere with NAAA activity, four mechanistically distinct analgesic and antiinflammatory drugs—gabapentin, ketamine, ketoprofen and morphine—failed to stop the conversion to CPLS when administered on PFD2-4 (FIGS. 5K-5L). Additionally, even though NAAA blockade attenuates inflammation in rodent models⁹, the 3-day ARN19702 regimen had no detectable influence on paw edema, likely owing to its brief duration. We interpret these findings as indicating that NAAA inhibitors halt the transition to pain chronicity through a mechanism that is distinguishable from their antinociceptive and antiinflammatory properties. To mark this distinction, we will refer to such mechanism as ‘algostatic’.

To determine whether the algostatic effects of NAAA inhibition could be generalized to other models of persistent pain, we asked whether ARN19702 might affect the development of sensory and cognitive abnormalities in mice subjected to surgical nerve injury²⁹. We loosely tied the sciatic nerve under anesthesia and, starting 24 h later, administered ARN19702 or its vehicle once daily for 3 days. Withdrawal responses and long-term memory were assessed on post-operative days 7-14 and 26, respectively. Treatment with ARN19702 prevented the appearance of both hypersensitivity and memory deficits, an effect that was phenocopied by genetic NAAA deletion. We conclude that post-injury NAAA blockade interrupts the emergence of chronic pain-like states elicited in mice by chemical or mechanical peripheral organ lesion.

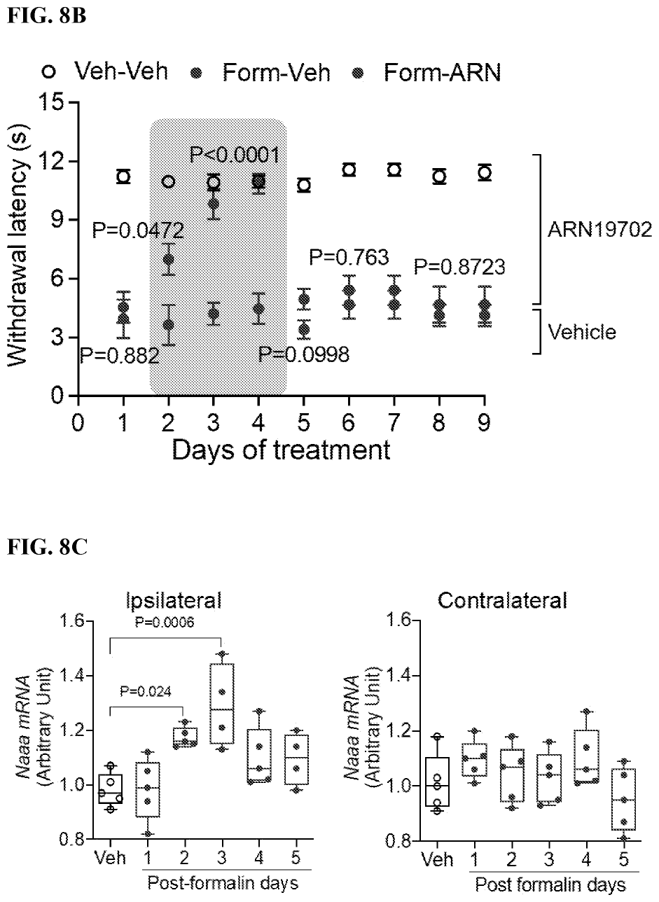

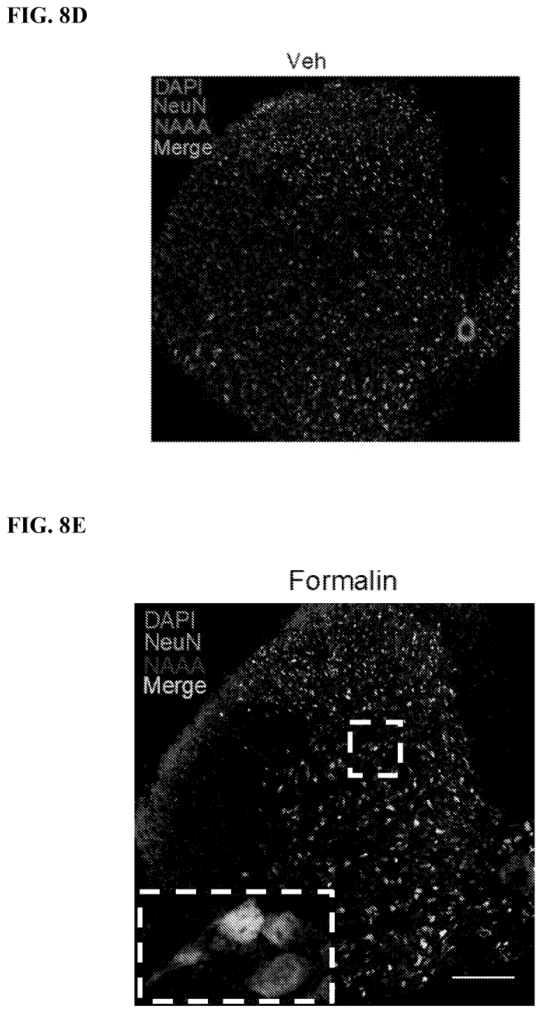

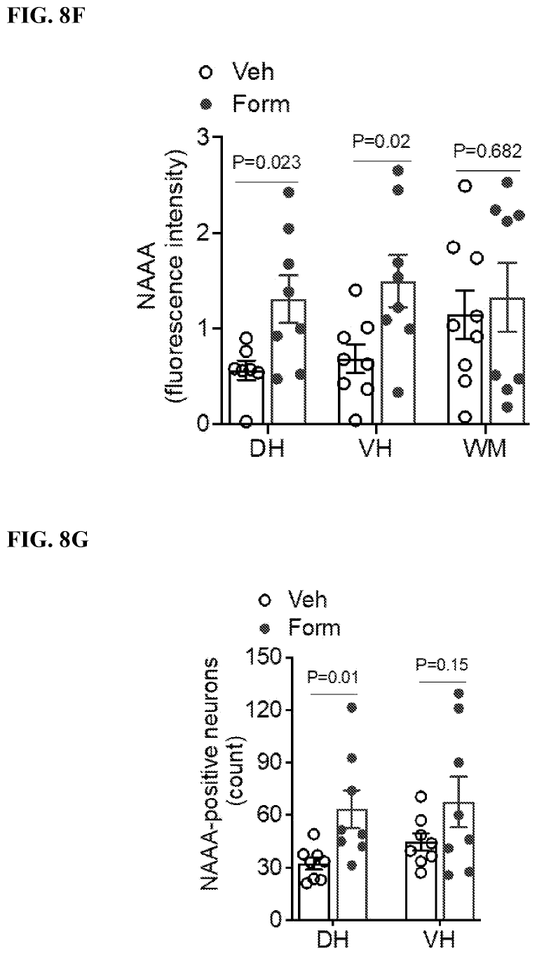

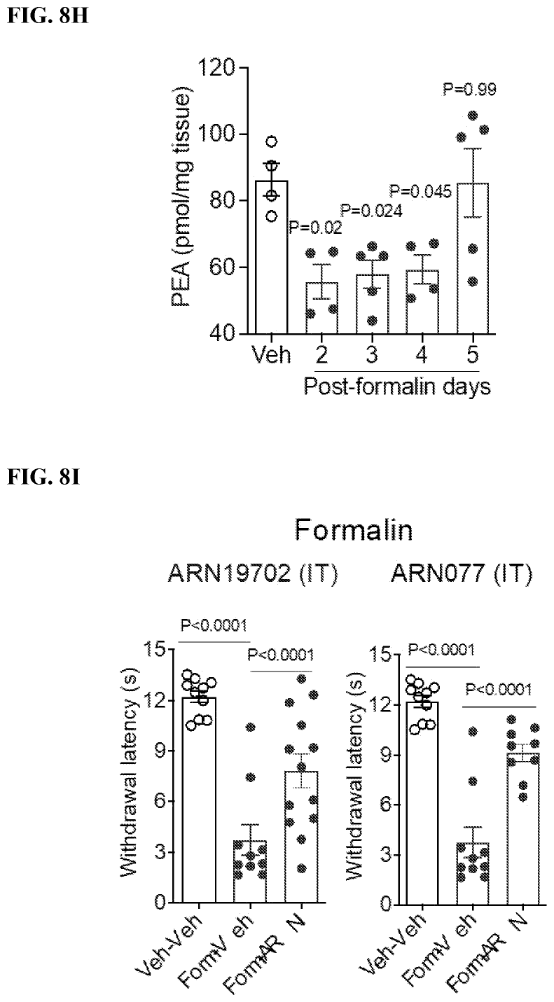

A critical period for pain chronification: The ability of NAAA inhibitors to stop CPLS consolidation was crucially dependent on the timing of their administration (FIGS. 8A-8B). ARN19702 provided full protection when given once on PFD3 or PFD4 but had no such effect when given on PFD1 or PFD5 to PFD9—even though at all time points it temporarily attenuated pain behaviors. Administration on PFD2 was partially effective. The time window for NAAA-inhibitor efficacy coincided with a transient rise in Naaa gene transcription in the lumbar (L4-L6) spinal hemicord ipsilateral to the injured paw, which was maximal at PFD3 (FIG. 8C). No change occurred on the contralateral side, where Naaa mRNA content remained stable from PFD1 to PFD5 (FIG. 8C). The spike in Naaa transcription was associated with higher levels of NAAA protein (FIGS. 8D-8F)—which was localized to neuronal and non-neuronal cells in both dorsal and ventral horns—and with a reduction in local PEA levels (FIG. 8H). Importantly, accrued spinal-cord NAAA activity was required for the induction of CPLS, which was stopped by post-injury intrathecal infusion of either ARN19702 or ARN077, a covalent NAAA inhibitor that cannot cross the blood-brain barrier⁹. In sum, the results show that end-organ damage sets off a transitory suppression of NAAA-regulated PEA signaling in innervating segments of the spinal cord, which starts 24 h after the injury, lasts approximately 72 h, and is required for pain to become chronic.

Transcriptomic and metabolomic experiments offered insights into the molecular events unfolding during this critical period. RNA sequencing analyses of L4-L6 spinal cord fragments ipsilateral to the formalin injection site identified large-scale transcriptional changes at PFD3 and PFD4, compared to time- and site-matched controls. At PDF4, when changes were largest, differentially upregulated transcripts were significantly enriched in the following Gene Ontology categories: synaptic membrane [adjusted P value (P_{adj})=1.17-35], neuron to neuron synapse (P_{adj}=7.82-30), postsynaptic membrane (P_{adj}=8.42⁻³⁰), glutamatergic synapse (P_{adj}=8.40⁻²⁷) and axon development (P_{adj}=2.53⁻²⁶). For example, expression of neuronal genes encoding for, among others, voltage-gated sodium channels (e.g., Sen1a and Sen8a), calcium channels (e.g., Cacn1a and Cacn1b) and α-amino-3-hydroxy-5-methyl-4-isoxazole propionic acid (AMPA) receptor-regulating proteins (e.g., Cacng2 and Cacng5) was strongly enhanced. Transcription of key components of cholesterol biosynthesis (e.g., Hmgcr, Hmgcs2)—a requisite for membrane biogenesis³⁰—was also heightened. These changes are consistent with evidence of widespread synaptic¹⁵ and proteomic³¹ modifications in spinal cord of animals experiencing chronic pain states. By contrast, differentially downregulated transcripts at PFD4 were enriched in categories including mitochondrial protein complex (P_{adj}=3.93-69), inner mitochondrial membrane protein complex (P_{adj}=4.03⁻⁴⁷) and mitochondrial respiratory chain (P_{adj}=2.01⁻³²). Accordingly, many components of tricarboxylic acid (TCA) cycle and oxidative phosphorylation were suppressed on PFD4. The opposite occurred, however, with two bifunctional 6-phosphofructo-2-kinase fructose-2,6-biphosphatase (Pfkfb) family members (Pfkfb3 and Pfkfb4) and with the glucose transporter Glut1 (Slc2a12). Of note, Pfkfb3 shunts glucose toward glycolysis whereas Pfkfb4 redirects glucose toward the pentose phosphate pathway (PPP), suggesting that both processes might be activated during the critical period for CPLS development³².

The transcriptional shift was paralleled by a largely concordant set of metabolomic changes. Decreased concentrations of free amino acids and non-esterified unsaturated fatty acids together with increased cholesterol and phospholipid precursors (zymosterol and sn-glycerol-3-phosphate, respectively) support the possibility that protein synthesis and membrane biogenesis are accelerated on PFD4. Concomitantly, N-acetyl-L-aspartate, which provides carbon units for neural lipid biosynthesis³³, was increased whereas urea cycle intermediates ornithine and citrulline declined, possibly reflecting accrued nitrogen recycling³⁴. Furthermore, elevated glucose, glucose-1-phosphate (GIP), glucose-6-phosphate (G6P), fructose-6-phosphate (F6P) and ribulose-5-phosphate (Ru5P) at PFD3 and/or PFD4 were suggestive of upregulated glucose transport, glycogenolysis (GIP), glycolysis (G6P, F6P) and the oxidative branch of PPP (Ru5P). The small but significant decrease in citric acid on PFD3 was consistent with reduced TCA cycle activity, while unchanged lactate levels suggested that glycolysis-derived pyruvate (which was undetectable in our samples) may have been utilized for biosynthetic needs. Together, the above alterations may account for the rise in 5′-adenosine monophosphate (AMP) and 5′-inosine monophosphate (IMP), two nucleotides that accumulate in cells during energy crises³⁵. Lastly, the neurotransmitters glutamate and 5-hydroxytryptamine were increased whereas γ-aminobutyric acid was decreased, possibly due to imbalanced excitation/inhibition and enhanced descending serotonergic input¹⁵. Thus, the critical period for CPLS consolidation coincides with a transcriptionally controlled metabolic switch from mitochondrial respiration to aerobic glycolysis, which is localized to spinal cord segments that receive direct nociceptive input from the lesioned paw. Systemic ARN19702 administration on PFD2-3 prevented the appearance of transcriptional changes on the following day, demonstrating a critical role for NAAA in enabling this switch.

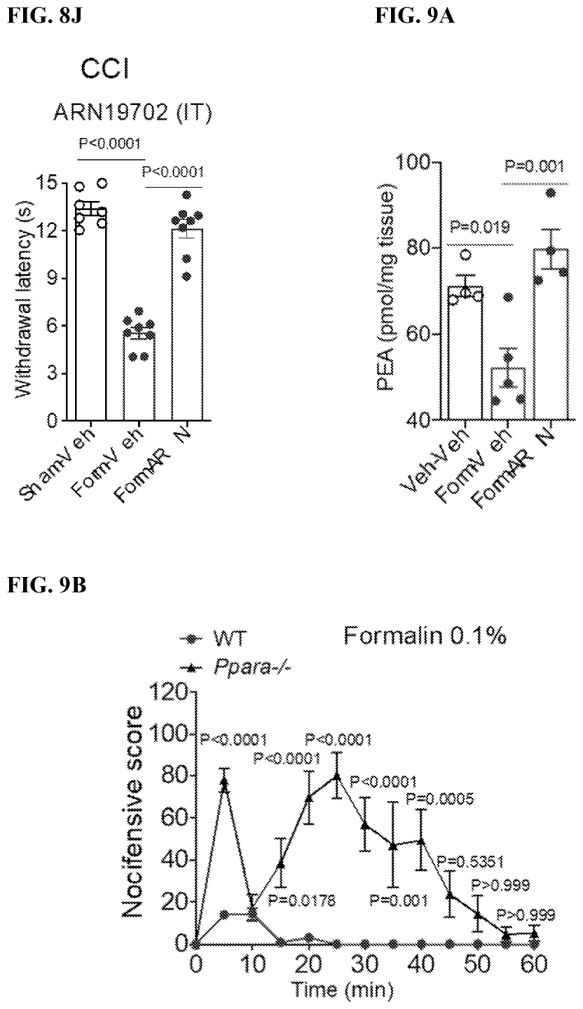

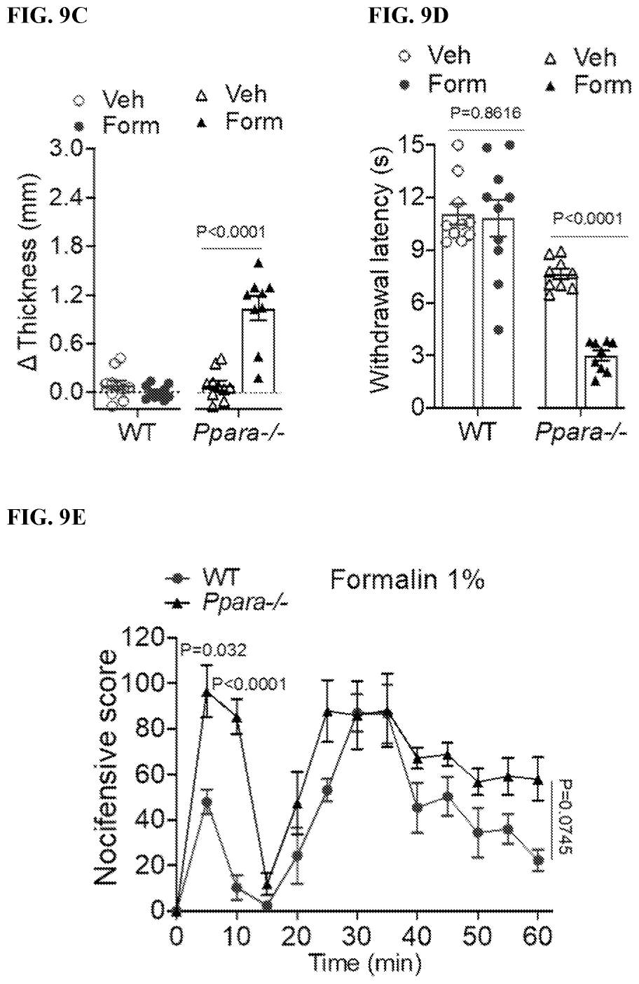

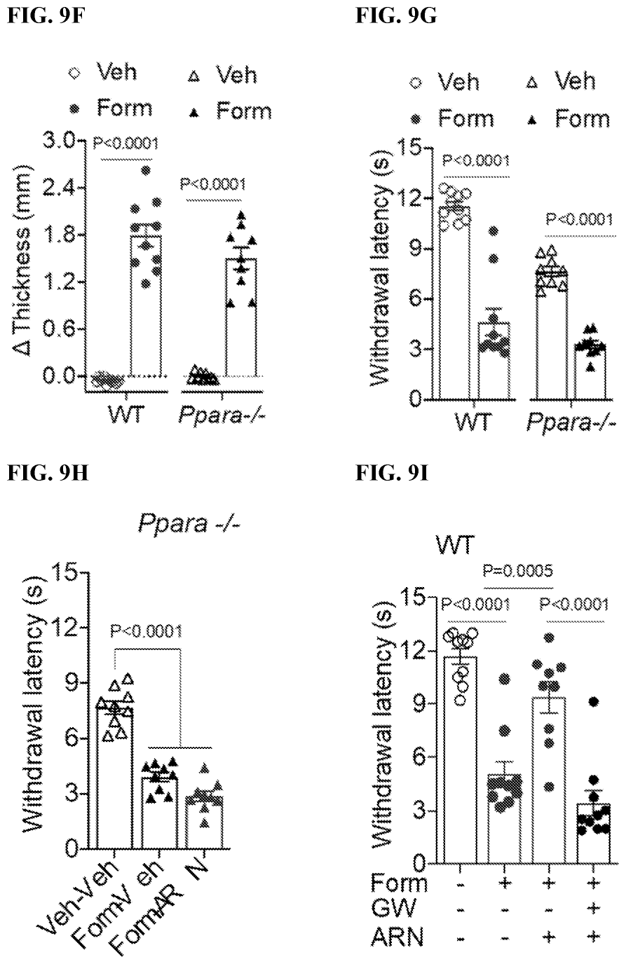

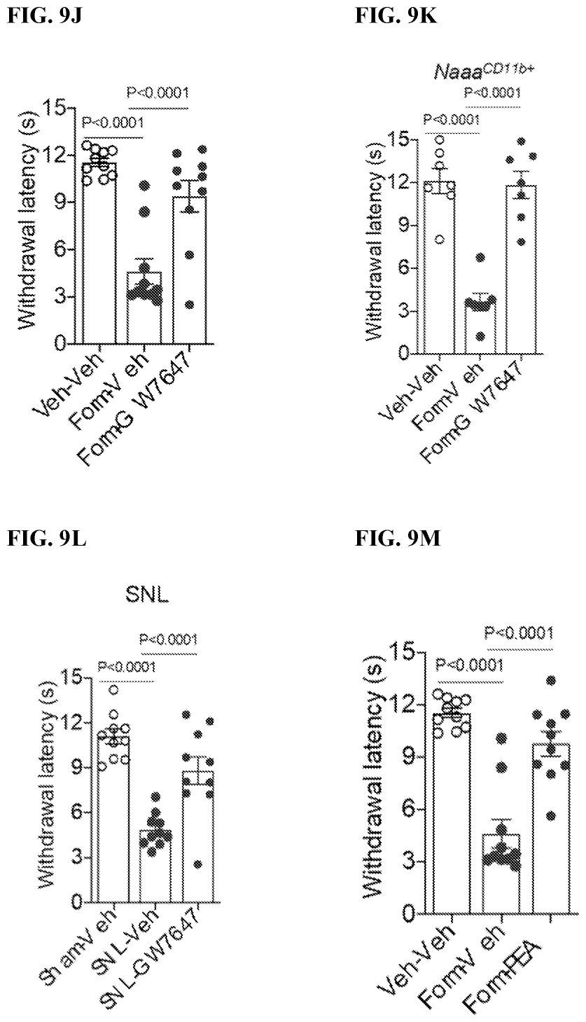

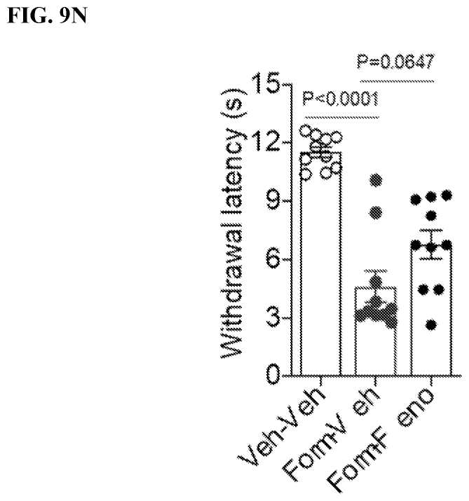

NAAA governs pain chronification via PEA signaling at PPAR-α: Post-formalin ARN19702 administration normalized PEA levels in ipsilateral lumbar spinal cord (FIG. 9A), suggesting that PPAR-α—which is expressed in central neurons^{10,36} and monocyte-derived cells¹¹, among others—might mediate the algostatic effects of NAAA inhibition. Experiments with mice constitutively lacking the nuclear receptor confirmed this possibility. When challenged with either 0.1% or 1% formalin, PPAR-α-null mice exhibited stronger nociception, compared to wild-type controls (FIG. 9B and FIG. 9E), which gave way at both doses to inflammation (FIG. 9C and FIG. 9F) and lasting hypersensitivity (FIG. 9D, FIG. 9G). Importantly, transition to CPLS in these mutants was unaffected by ARN19702 (FIG. 9H), a trait that was replicated in wild-type animals by pretreatment with the selective PPAR-α antagonist GW6471 (FIG. 9I). Further supporting a pivotal role for PPAR-α in gating pain chronification, we found that post-injury administration of the agonist GW7647 stopped this process in three different settings: (i) wild-type mice treated with 1% formalin (FIG. 9J); (ii) NAAA-overexpressing mice treated with 0.1% formalin (FIG. 5K); and (iii) wild-type mice subjected to sciatic nerve ligation (FIG. 9L). Like NAAA inhibition, PPAR-α activation by GW7647 prevented injury-associated changes in spinal-cord transcription. Finally, CPLS was blocked by exogenous PEA but not by the weak PPAR-α agonist fenofibrate (FIGS. 9M-9N).

Discussion: Traumatic nerve damage, a major risk factor for chronic pain development¹, poses an extraordinary challenge to first- and second-order nociceptive neurons, as it pressures them to enact large-scale neuroplastic adaptations while simultaneously maintaining energy homeostasis along the considerable distance of their axonal trees (>1m in humans)37. Finite neuronal resources must be allocated to two equally urgent tasks: heightened energy production to sustain the upsurge in neural activity caused by the lesion, and biomass generation to effect the structural changes needed to support peripheral and central sensitization¹⁵. The results of our transcriptomic and metabolomic experiments suggest that mouse spinal cord neurons negotiate this conflict—most likely in concert with cells of other lineages^{38,39}—by temporarily shifting ATP production from mitochondrial respiration, which is energetically efficient but has substantial proteomic costs⁴⁰, to Warburg-like aerobic glycolysis¹⁶, which is far less efficient but can generate carbon units for protein and lipid biosynthesis. A maladaptive consequence of this metabolic switch, whose occurrence in differentiating⁴¹ and acutely stimulated adult neurons^{42,43} is well documented, is the instigation of an energy crisis which might disrupt electrochemical gradients and ultimately precipitate central sensitization.

Two parallels are worth highlighting. The first is offered by macrophages, which shift their metabolism toward aerobic glycolysis when transitioning to trained immunity⁴⁴, a sensitized state that heightens both their responsiveness to recurring infections and the risk of unleashing a dangerous hyperinflammatory reaction⁴⁵. This phenotype shift is stopped by the anti-diabetic drug metformin45, which also prevents experimental neuropathic pain in male (but not female) rodents⁴⁶. In both instances, metformin may work by indirectly recruiting AMP-activated protein kinase, which reacts to cellular energy crises by stimulating ATP generation and suppressing anabolic processes⁴⁷. A second parallel can be drawn with primary afferent sensory neurons exposed to chemotherapeutic and antiviral drugs or to hyperglycemia, which cause painful peripheral neuropathies through mechanisms that likely involve a shared ability to impair neuronal mitochondrial function⁷. In this case also, metformin may exhibit therapeutic efficacy, as shown in a male rat model of type-1 diabetes⁴⁸. Thus, despite their dissimilarities, these diverse responses to noxious signals appear to rely on a shared proteome allocation strategy that transiently favors aerobic glycolysis over oxidative phosphorylation—a suboptimal trade-off⁴⁹ that may render affected cells persistently hypersensitive to innocuous stimuli. A diverse set of preexisting factors—including sex, prior injury, systemic metabolic status and stress resilience²—might either aggravate or mitigate the risks posed by such strategy.

Preclinical and clinical studies have identified NAAA-regulated PEA signaling as a potential target for analgesic and antiinflammatory therapy^{9,12}. The present results show that this signaling complex governs the progression to chronic pain not by modulating nociception or inflammation but rather by serving, at a critical juncture of this consolidation process, as a regulatory checkpoint for spinal cord metabolism, a hypothesis that is fully consistent with the ubiquitous role of PPAR-α in the control of lipid and glucose utilization¹¹. Thus, NAAA inhibitors exemplify a novel class of pharmacological agents—distinct from classical analgesics and anti-inflammatories and possibly including metformin—which may be able to forestall the transition from acute to chronic pain.

### Example 2: Methods

Data reporting: Experimental subjects were randomized and data analyses were performed under blinded conditions. Sample size was not predetermined.

Data availability: Data supporting the findings of this study are available within the paper and its supplementary information files.

Chemicals: ARN19702 and ARN077 were synthesized following established procedures^{50,51}. ARN16186 was prepared as described⁵². Briefly, sodium borohydride reduction of tert-butyl 3-oxo-8-azabicyclo[3.2.1]octane-8-carboxylate gave an endo/exo mixture of the corresponding 3-hydroxy isomers which were separated by column chromatography. Etherification of the (exo)-3-hydroxy isomer with 4-butylphenol under Mitsunobu conditions, followed by N-Boc deprotection (TFA/DCM, 1:3) yielded the corresponding (endo)-3-(4-butylphenoxy)-8-azabicyclo[3.2.1]octane, which was then submitted to N-sulfonylation with 3,5-dimethyl-1H-pyrazole-4-sulfonyl chloride, in the presence of sodium hydride, to yield ARN16186. Formalin, capsaicin, morphine sulfate, gabapentin, ketamine hydrochloride (solution, 100 mg-ml⁻¹), ketoprofen, GW7647, GW6471 and fenofibrate were from Sigma-Aldrich (St. Louis, USA). A proprietary water-soluble PEA formulation (Levagen Plus™) was manufactured at Shilpa Medicare Ltd (Raichur, India) and was a generous gift of Gencor (Irvine, CA). [2H4]-PEA was from Cayman Chemicals (Ann Harbor, USA). All solvents and chemicals were of the highest available grade.

Animals: We used C57BL/6J mice (10 to 12-week-old, 24-26 g) unless otherwise indicated. Ppar^{tniJGonz}-targeted mutation (Ppara^{-/-}) mice were purchased from Jackson Laboratory (Bar Harbor, ME, USA). Naaa mice (B6N-A^{tmlBrd}Naaa^{tmla(KOMP)wtsi}/WtsiH) were acquired from MRC Harwell (Didcot, UK) through the EMMA-European Mouse Mutants Archive⁵³. This mouse line was generated as a knockout first, reporter-tagged insertion with conditional potential allele. Naaa^{-/-} mice were obtained by mating animals heterozygous for allele. NAAA deletion was confirmed by RT-PCR, Western blot and enzyme activity measurements, as described⁵⁴. Mice overexpressing NAAA in CD11b-positive monocyte-derived cells (Naaa^{CD11b+}) were generated as follows⁵⁴. Heterozygous NAAA conditional knock-in mice were produced at GenoWay (Lyon, France) by targeted insertion of the Naaa gene within the Rosa26 locus using GenoWay's Quick Knock-In™ technology. A loxP flanked transcriptional stop cassette was incorporated between the transgene and the synthetic CAGG promoter to allow the expression of the resulting transgene to be dependent upon Cre recombinase. These ‘Rosa26 knock-in’ mice were used to produce homozygous animals, which were then cross-bred with CD11b-Cre mice (Jackson Labs) to generate the Naaa^{CD11b+} line. The mice were maintained in a pathogen free-environment on 12-hr light/dark cycle at controlled temperature (22° C.) and humidity (50-60%). Food and water were available ad libitum.

Animals were randomly assigned to treatment groups and, before the start of the experiments, were handled for 3 consecutive days (˜3 min per animal/day) and behavioral testing was conducted during the light phase of the light/dark cycle. Efforts were made to minimize the number of animals used and their discomfort. The study complied with all ethical regulations of the National Institutes of Health (NIH) Guide of the Care and Use of Laboratory Animals and the recommendation of the International Association for the Study of Pain. Experimental procedures were approved by the Animal Care and Use Committee of the University of California, Irvine.

Drug administration: Drug solutions were prepared shortly before use. Capsaicin was dissolved in a vehicle of Tween 80-distilled water (7/93, vol), sonicated and filtered before intraplantar injection. Gabapentin, morphine and PEA were dissolved in distilled water. Gabapentin was administered by intraperitoneal (IP) injection, morphine and PEA by subcutaneous (SC) injection. Ketoprofen, ARN19702, ARN16186, ARN077, GW7647, GW6471 and fenofibrate were dissolved in polyethylene glycol 400/Tween-80/distilled water (15/15/70, vol) and administered by IP or intrathecal (IT) injection. An appropriate volume of the commercial ketamine solution was administered by the IP route. For IT administration, the mice were anesthetized with isoflurane (3-5% in oxygen). Percutaneous injections were made at intervertebral space L5-L6 with a 30-gauge needle perpendicular to the skin. Injectate volumes of 5 μl were delivered using a 10 μl Hamilton syringe and placement was confirmed by a lateral tail flick as the needle entered the subarachnoid space.

RNA isolation and quantitative RT-PCR: We extracted total RNA from lumbar spinal cord fragments (L4-L6) ipsilateral and contralateral to the formalin injection site using the TRIzol™ reagent (Thermo Fisher Scientific, Waltham, USA). RNA was purified with PureLink™ RNA Mini Kit (Invitrogen, Carlsbad, USA). Prior to purification, samples were rendered genomic DNA-free by passing the isolated RNA through a gDNA Eliminator spin column (Qiagen, Germantown, USA). RNA concentration and purity were determined using a NanoDrop 2000/2000-c spectrophotometer (Thermo Fisher). cDNA was synthesized using 2 μg of total RNA as input for the High-Capacity cDNA RT Kit with RNase Inhibitor (Applied BioSystems, Foster City, USA) with a final reaction volume of 20 μL. First-strand cDNA was amplified using TaqMan™ Universal PCR Master Mix (Thermo Fisher) following manufacturer's instructions. Real-time PCR primers and fluorogenic probes were purchased from Applied Biosystems (TaqMan® Gene Expression Assays). We used TaqMan gene expression assays for mouse Actin-β (Mm00607939_s1), Hprt (Mm00446968_m1), Gapdh (Mm99999915_g1) and Naaa (Mm01341699_m1) (Applied Biosystems). Real-time PCR reactions were performed in 96-well plates using CFX96™ Real-Time System (Bio-Rad, Hercules, USA). The Bestkeeper software was used to determine the expression stability and the geometric mean of three different housekeeping genes (Actb, Hprt and Gapdh). The relative quantity of genes of interest was calculated by the 2^{-ΔΔCt} method and expressed as fold change over vehicle controls.

Lipid extraction: Lumbar L4-L6 spinal cord fragments (˜15 mg each) were transferred into 2 ml Precellys® soft tissue tubes (Bertin Instruments, France) and diluted with ice-cold acetone (1 ml) containing [2H4]-PEA (50 μl, 100 nM) Samples were homogenized at 4° C., 6000 rpm, 15 s/cycle for 2 cycles with 20 s pause in between cycles. Supernatants were transferred into 8-ml glass vials and dried under nitrogen. Chloroform/methanol (2/1, vol, 3 ml) and water (1 ml) were added to the samples, which were then stirred vigorously and centrifuged at 830×g for 15 min at 4° C. The lower (organic) phases were collected and upper phases were extracted again with chloroform (2 ml) and combined with the previous extract. Extracts were dried under nitrogen, reconstituted in acetonitrile (0.1 ml), transferred to deactivated glass inserts and placed inside amber glass vials for liquid chromatography/tandem mass spectrometry (LC/MS-MS) analyses.

PEA quantification: PEA was fractionated using a 1260 series LC system (Agilent Technologies, Santa Clara, CA) coupled to a 6460C triple-quadrupole mass spectrometric detector (MSD; Agilent). A step-gradient separation was performed on an Eclipse XDB C18 column (1.8 μm, 2.1×30.0 or 50.0 mm; Agilent) with a mobile phase consisting of water containing 0.25% acetic acid and 5 mM ammonium acetate as solvent A and methanol containing 0.25% acetic acid and 5 mM ammonium acetate as solvent B. The gradient conditions were as follows: starting at 78% B to 28% B in 8.00 min, changed to 95% B at 8.01 min, and maintained till 10.00 min; then changed back to 78% B at 10.01 min; the equilibration time was 5 min. The flow rate was 0.3-0.5 ml-min-1. The autosampler was maintained at 9° C. and the column at 40° C. Injection volume was 2 μl. To prevent carry over, the needle was washed 3 times in the autosampler port for 30 s before each injection, using a wash solution consisting of 10% acetone in water/methanol/isopropanol/acetonitrile (1/1/1/1, vol). The MSD was operated in the positive electrospray ionization mode, and analytes were quantified by multiple reaction monitoring. Capillary and nozzle voltages were 3500 V and 300-500 V, respectively. Drying gas temperature was 300-350° C. with a flow of 9.0-11.0 l/min. Sheath gas temperature was 300-375° C. with a flow of 12 l-min⁻¹. Nebulizer pressure was set at 45-50 psi. We used the MassHunter software (Agilent) for instrument control, data acquisition and analysis.

RNA sequencing and bioinformatics analysis: We extracted total RNA with the RNeasy Mini Kit (Qiagen). Samples with RNA integrity number ≥8.5 were kept for library construction. cDNA synthesis, amplification, library construction, and sequencing were performed at Novogene (Bejing, China) using the Illumina NovaSeq platform with paired-end 150 bp (PE 150) sequencing strategy. Downstream analysis was performed using a combination of programs including STAR, HTseq, Cufflink and Novogene's wrapped scripts. Alignments were parsed using the STAR program and differential expressions were determined using DESeq2/edgeR. Gene Ontology (GO) and Kyoto Encyclopedia of Genes and Genomes (KEGG) enrichment were implemented using the ClusterProfiler. GO terms with adjusted P value less than 0.05 were considered significantly enriched in differential expressed genes.

Metabolomic analysis: Metabolomics analyses were performed at the West Coast Metabolomics Center of the University of California Davis, as described⁵⁵. Briefly, frozen lumbar spinal cord (L4-L6) fragments (˜5 mg) were homogenized in Eppendorf tubes (2 ml) for 2 min using metal spheres (20 mm diameter) in a MM300 mill (Retsch, Germany). A mixture of isopropanol/acetonitrile/water (3/3/2, vol) was added to the grounded tissue (1 ml per 20 mg), stirred for 10 s and shaken at 4° C. for 5 min. After centrifugation at 14,000×g for 2 min, the supernatant was collected and concentrated to dryness in a CentriVap Cold Traps vacuum concentrator (Labconco, Kansas City, USA) at room temperature for 4 h. Derivatization for gas chromatography time-of-flight mass spectrometry was carried out as described⁵⁶. Metabolites were identified based on retention time and spectral properties, using the BinBase algorithm, and were matched against an internal mass spectral library (http://fiehnlab.ucdavis.edu/). They were reported if present in at least 80% of the samples.

General histological procedures: Mice were anaesthetized with isoflurane, transcardially flushed with cold phosphate-buffered saline (PBS) and perfused with 4% paraformaldehyde (PFA) in PBS. Spinal cords were extruded, post-fixed in PFA/PBS for 24 h, and cryoprotected in sucrose (30% in PBS) at 4° C. Three to six series of transverse sections (40 μm thickness) were collected using a cryostat and stored at −20° C.

Confocal microscopy: Double and triple immunostaining experiments were performed by sequential incubation with primary antibodies [Anti-NAAA (Invitrogen, Carlsbad, USA; PA5-69357); Anti-NeuN (Cell Signaling Technology, Danvers, USA; clone E4M5P #94403)] followed by appropriate secondary Alexa Fluor antibodies (1:1000; Invitrogen). Validation of the anti-NAAA antibody is provided in FIGS. 14A-14D. Images were collected using an Olympus (Tokyo, Japan) FV3000 confocal microscope with a 10/40×0.4/1.25 numerical aperture objective lens. Quantification of fluorescence intensity was performed using the NIH Fuji Is Just ImageJ (FIJI) software on a minimum of 3 images per animal.

Fos immunohistochemistry: Two weeks after formalin injection, the hind paw contralateral to the formalin injection site was challenged with capsaicin (0.1 μg, intraplantar). Two h later, the mice were euthanized and perfused with ice-cold PBS and then with 4% PAF in PBS, and the lumbar spinal cord was removed and processed as described above. Free-floating sections (30 μm) were immersed in PBS containing hydrogen peroxide (0.3%) and non-specific binding was removed by incubation (1 h) with 5% goat serum and 0.3% Triton X-100 in 0.1 M PBS, followed by incubation (48 h) at 4° C. with a rabbit anti-c-Fos antibody (1:1000, #2250, Cell Signaling Technology). The tissue was incubated in the presence of biotinylated goat anti-rabbit IgG followed by Vectastain Elite ABC reagent (1:600, #PK6101, Vector Laboratories, Burlingame, USA). Fos immunoreactivity was visualized with the avidin-biotin peroxidase method using diaminobenzidine as chromogen. Sections were washed with double-distilled water, slide-mounted, air-dried, dehydrated, and coverslipped with Permount™. Slides were scanned using a Leica (Wetzlar, Germany) DM6B light microscope and 4 to 6 sections per subject exhibiting the highest number of Fos-immunoreactive cells, based upon qualitative evaluation, were imaged at 10× magnification. Fos-expressing nuclei were counted using a computer-assisted image analysis system (LAS AF 2D, Leica) by an observer blinded to experimental conditions.

### Behavioral Tests

Formalin: We injected formalin (0.1, 0.3, 1 and 3% vol, 20 μl) or saline into the plantar surface of the right hind paw. Following injection, the mice were immediately transferred to a transparent observation chamber where nocifensive behavior (time spent licking or biting the injected paw, number of paw shakings) was videorecorded for 60 min and quantified by blinded observer. Mechanical allodynia, heat hyperalgesia and paw edema were measured on post-formalin day (PFD) 7, 14, 21, 60 and 120 in both injected and non-injected paws.

Capsaicin: Capsaicin or its vehicle was injected into the plantar surface of the left hind paw (20 μl) or left forepaw (10 μl), i.e. contralaterally to the formalin injection site. Nocifensive behavior was videorecorded for 10 min and evaluated under blinded conditions. Outcome measures after hind-paw injection included: time spent licking or biting the injected paw and number of paw-shaking episodes⁵⁷; outcome measures after forepaw injection also included number of paw-rubbing episodes (Supplementary video).

Sciatic nerve ligation (SNL): SNL surgeries were carried out as described⁵⁸. Briefly, the mice were anesthetized with isoflurane and the right common sciatic nerve was exposed at the level of the middle thigh by blunt dissection under aseptic conditions. Proximal to the trifurcation, the nerve was cleaned from surrounding connective tissue and 3 chromic cat gut ligatures (4-0, Ethicon, Somerville, USA) were loosely tied around it at 1-mm intervals. The wound was closed with a single muscle suture and skin clips. In sham-operated animals, the nerve was exposed but not tied.

Mechanical allodynia was assessed using a dynamic plantar aesthesiometer (Ugo Basile, Comerio, Italy). After a 45-min habituation period in transparent cages positioned on a wire mesh surface, a mechanical stimulus was applied to the plantar surface of both hind paws by an automated steel filament exerting an increasing force ranging from 0 to 5 g over 10 s. Three withdrawal thresholds (in g) were recorded and averaged.

Paw edema was measured with a digital caliper (Fisher Scientific, USA) and is expressed as the difference (A paw thickness, mm) between ipsilateral and contralateral paws.

Thermal hyperalgesia was measured using a Hargreaves plantar test apparatus (San Diego Instruments, San Diego, USA). After a 45-min habituation period, the plantar surface of both hind paws was exposed to a beam of radiant heat through the glass floor. The cut-off time was set at 15 s. The stimulation was repeated 3 times with an interval of 2 min between stimuli and latencies (in s) to withdraw the paw were recorded and averaged.

Elevated plus maze test: We placed each mouse in the central platform of the maze, facing the open arm opposite to the experimenter, and behavior was recorded using the Debut video capture software (NCH Software, Canberra, Australia). A blinded observer measured the amount of time spent in the open and closed arms, as well as the number of open and closed arm entries. The open arms of the maze were illuminated at 150-170 lux and the closed arms at 40-50 lux.

Novel Object Recognition test was performed as described⁵⁹. The test was conducted over 3 days. On day 1, mice were habituated to the empty arena for 20 min. On day 2, they were returned to the arena, which now contained 2 identical objects. On day 3, one of the objects was replaced with another one of different shape, color and texture. Mice were allowed to explore the arena for 10 min, and the total time spent exploring each object (i.e., nosing and sniffing at a distance ≤2 cm) was recorded by a blinded observer.

Feeding and motor activity: We habituated animals to the test cages for 3 days prior to trials. Food intake and motor activity were recorded for 24 h using an automated system (TSE, Bad Homburg, Germany). The system, protocol and feeding parameters surveyed were previously described⁶⁰. Motor activity was recorded using an X-Y matrix of infrared sensors and is reported as number of beam breaks in 24 h.

Magnetic Resonance Imaging (MRI) acquisition: Mice were anesthetized, flushed intracardially with ice-cold PBS and then with 4% PAF in PBS (pH 7.4). Brains and spinal cords were removed within their osseous structures and post-fixed overnight in 4% PAF, followed by three 5-min washes with PBS the next day, and stored in PBS (pH 7.4) containing sodium azide (0.02%, weight) at 4ºC. Ex vivo diffusion tensor imaging (DTI) and T2 weighted imaging (T2WI) studies were performed using a 9.4 T Bruker Avance imager (Bruker Biospin, Billerica, USA). Data were acquired with the following parameters: 1.5 cm field of view, 0.5 mm slice thickness, and a 128×128 acquisition matrix and zero filled to 256×256. DTI parameters were as follows: repetition time (TR)/echo time (TE)=8000 ms/35.66 ms, 30 isolinear directions, B=3000 mT/m, and 5 B0 images acquired prior to weighted images. T2WI parameters were TR/TE=4000/10 ms with 10 equally spaced echoes.

### MRI Processing and Analyses

Brain: We used 3D automatic registration for unbiased region parcellation to analyze DTI metrics in brain structures. Scans underwent eddy current correction and brain was digitally removed from surrounding skull and muscles using 3D Pulse-Coupled Neural Networks (PCNN3D v1.2) in MATLAB R2017a (MathWorks). Extraction masks were reviewed and adjusted by a blinded experimenter using ITK Snap (version 3.8.0). T2 maps were generated using FMRIB's Software Library (FSL v5.0; FMRIB, Oxford, UK). FMRIB's Diffusion Toolbox was used to generate parametric DTI maps, in which a diffusion tensor model was fit at each voxel61. The Australian Mouse Brain Atlas Consortium (AMBMC) atlas⁶² was fit to each individual animal and regional labels were applied with Advanced Normalization Tools (ANTs). Regional statistics for T2 values, fractional anisotropy (FA), mean diffusivity (MD), axial diffusivity (AD), and radial diffusivity (RD) were extracted for 80 bilateral regions. Regional volumes were extracted from T2 scans. This robust method allows an unbiased and rapid segmentation of GM and WM regions from the mouse brain.

Spinal cord: MRI scans were processed for eddy current correction as outlined above, and the scans were manually masked and extracted using ITK Snap (version 3.8.0). Masks were reviewed and adjusted by a blinded experimenter. FMRIB's Diffusion Toolbox was used to generate parametric DTI maps. The resultant maps were then analyzed in DSI studio (http://dsi-studio.labsolver.org). Distinct regions of interest throughout the spinal cord were drawn manually [see ref.⁶³ for accurate identification of spinal cord segments] for each animal without right/left distinction for white and gray matter. Regional average value of the diffusion indices (FA, MD, AD, RD) and volume were automatically extracted for each spinal cord segments.

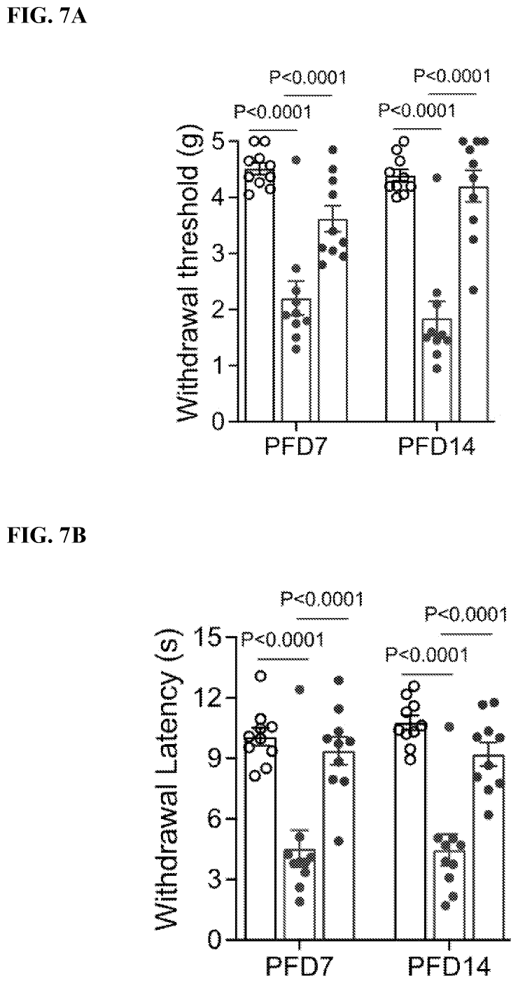

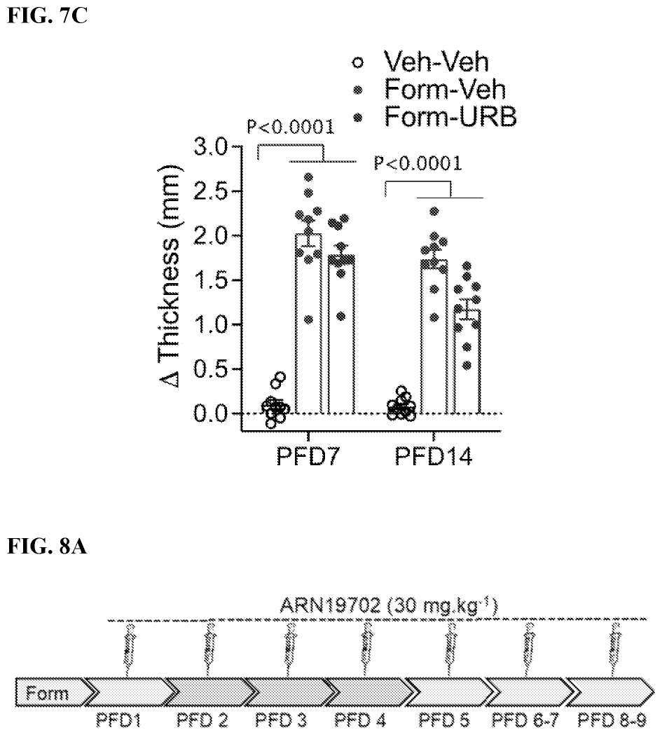

Statistical analyses: Data are presented as mean±S.E.M. Statistical significance was determined using unpaired two-tailed Student's t test (with or without Bonferroni's correction) or ANOVA (one-way, two-way and multi-factorial) followed by Dunnett's or Bonferroni's post hoc test, as appropriate. Analyses were performed with either GraphPad Prism version 8.0 (GraphPad Prism) or the Statistical Package for Social Science program SPSS® (SPSS). The D'Agostino-Pearson normality test was used to determine the distribution of the population of formalin-injected mice (FIG. 1; FIGS. 71-7K). A k-means cluster analysis with 10 iterations followed by one-way ANOVA separated two groups of mice, which were termed ‘resilient’ and ‘vulnerable’ based on their enduring response to formalin.

### Example 3: List of Abbreviations

TABLE 1
Anatomical abbreviations of brain regions
analyzed by diffusion tensor imaging

| Abbreviation | Region |
|---|---|
| IL | Infralimbic cortex |
| PrL | Prelimbic cortex |
| OFC | Orbito-frontal cortex |
| FrC | Frontal cortex |
| TT | Tenia tecta |
| ACC | Anterior cingulate cortex |
| RSC | Retrosplenial cortex |
| M1, 2 | Primary and secondary motor cortices |
| S1, 2 | Primary and secondary somatosensory cortices |
| PPC | Posterior parietal cortex |
| V1, 2 | Primary and secondary visual cortices |
| Cl | Claustrum |
| Au | Auditory cortex |
| Ect | Ectorhinal cortex |
| PRh | Perirhinal cortex |
| Pir | Piriform cortex |
| Ent | Enthorinal cortex |
| SuC | Subicular complex |
| AS | Associated structure |
| BLA | Basolateral amygdala |
| CA1 | Cornus Ammonis 1 |
| CA2 | Cornus Ammonis 2 |
| CA3 | Cornus Ammonis 3 |
| DG | Dentate gyrus |
| Acb | Nucleus accumbens |
| Tu | Olfactory tubercle |
| nDBB | Nucleus of the diagonal band of Broca |
| CS | Corpus striatum |
| VP | Ventral Pallidum |
| BNST | Bed Nucleus of the stria terminalis |
| LV | Lateral ventricle |
| Den | Diencephalon |
| Cb | Cerebellum |
| CC | Corpus callosum |
| Cg | Cingulum |
| DHf | Dorsal hippocampal fissure |
| DF | Dorsal fornix |
| LO | Lateral olfactory tract |
| AC | Anterior commissure |
| FG | Fiber groups |

### REFERENCES FOR EXAMPLES 1-3

1. Chapman, C. R., & Vierck, C. J. J. Pain 18, 359.el-359.e38 (2017). 2. Denk, F., McMahon, S. B., & Tracey, I. Nat. Neurosci. 17, 192-200 (2014). 3. Basbaum, A. I., Bautista, D. M., Scherrer, G., & Julius, D. Cell 139, 267-284 (2009). 4. Ji, R. R., Chamessian, A., & Zhang, Y. Q. Science 354, 572-577 (2016). 5. Price, T. J. et al. Nat. Rev. Neurosci. 19, 383-384 (2018). 6. Joseph, E. K., & Levine, J. D. Pain 121, 105-114 (2006). 7. Bennett, G. J., Doyle, T., & Salvemini, D. Nat. Rev. Neurol. 10, 326-336 (2014). 8. Tsuboi, K., Takezaki, N., & Ueda, N. Chem. Biodivers. 4, 1914-1925 (2007). 9. Piomelli, D. et al. J. Med. Chem. 63, 7475-7490 (2020). 10. LoVerme, J. et al. J. Pharmacol. Exp. Ther. 319, 1051-1061 (2006). 11. Bougarne, N. et al. Endocr. Rev. 39, 760-802 (2018). 12. Artukoglu, B. B., Beyer, C., Zuloff-Shani, A., Brener, E., & Bloch, M. H. Pain physician 20, 353-362 (2017). 13. Rankin, L., & Fowler, C. J. Int. J. Mol. Sci. 21, 7942 (2020). 14. Annunziata, C. et al. FASEB J. 34, 350-364 (2020). 15. Latremoliere, A., & Woolf, C. J. J. Pain 10, 895-926 (2009). 16. Liberti, M. V., & Locasale, J. W. Trends Biochem. Sci. 41, 211-218 (2016). 17. Tjølsen, A., Berge, O. G., Hunskaar, S., Rosland, J. H., & Hole, K. Pain 51, 5-17 (1992). 18. Arendt-Nielsen, L. Central Sensitization in Humans: Assessment and Pharmacology. In Handbook of Experimental Pharmacology, vol. 227, ed. H.-G. Schaible, Springer, Berlin and Heidelberg, 2004. 19. Kaushal, M., Shabani, S., Budde, M., & Kurpad, S. J. Neurotrauma 36, 2279-2286 (2019). 20. Schlegel, A. A., Rudelson, J. J., & Tse, P. U. J. Cogn. Neurosci. 24, 1664-1670 (2012). 21. Baliki, M. N., & Apkarian, A. V. Neuron 87, 474-491 (2015). 22. Shiers, S. et al. J. Neurosci. 38, 7337-7350 (2018). 23. Eichenbaum H. Nat. Rev. Neurosci. 18, 547-558 (2017). 24. Lehmann, M. L., & Herkenham, M. J. Neurosci. 31, 6159-6173 (2011). 25. Kataoka, N., Shima, Y., Nakajima, K., & Nakamura, K. Science 367, 1105-1112 (2020). 26. Hitti, F. L., & Siegelbaum, S. A. Nature 508, 88-92 (2014). 27. Ji, G, & Neugebauer, V. Neurophysiol. 102, 2253-2264 (2009). 28. Sasso, O. et al. J. Invest. Dermatol. 138, 562-569 (2018). 29. Bennett, G. J., & Xie, Y K. Pain 33, 87-107 (1988). 30. Nohturfft, A., & Zhang, S. C. Annu. Rev. Cell Dev. Biol. 25, 539-566 (2009). 31. Gomez-Varela, D., Barry, A. M., & Schmidt, M. J. Proteomics 190, 1-11 (2019). 32. Yi, M. et al. Mol Metab 20, 1-13 (2019). 33. Clark J. B. Dev. Neurosci. 20, 271-276 (1998). 34. Spinelli, J. B. et al. Science 358, 941-946 (2017). 35. Hancock, C. R., Brault, J. J., & Terjung, R. L. J. Physiol. Pharmacol. 57, 17-29 (2006). 36. Warden, A. et al. Sci. Rep. 6, 27618 (2016). 37. Misgeld, T., & Schwarz, T. L. Neuron 96, 651-666 (2017). 38. Hayakawa, K. et al. Nature 535, 551-555 (2016). 39. Salter, M. W., & Stevens, B. Nat. Med. 23, 1018-1027 (2017). 40. Basan, M. et al Nature 528, 99-104 (2015). 41. Zheng, X. et al. eLife 5, e13374 (2016). 42. Yellen G. J. Cell Biol. 217, 2235-2246 (2018). 43. Bas-Orth, C., Tan, Y. W., Lau, D., & Bading, H. J. Biol. Chem. 292, 5183-5194 (2017). 44. Cheng, S. C. et al. Science 345, 1250684 (2014). 45. Netea, M. G. et al. Science 352, aaf1098 (2016). 46. Inyang, K. E. et al. Pharmacol. Res. 139, 1-16 (2019). 47. Hardie D. G. Int. J. Obes. (Lond) 32, S7-S12 (2008). 48. Ma, J. et al. Eur. J. Pharmacol. 764, 599-606 (2015). 49. Basan, M. et al. Nature, 584, 470-474 (2020). 50. Migliore, M. D. et al. Angew Chem Int Ed Engl 55, 11193-11197 (2016). 51. Sasso, O. et al. Pain 154, 350-360 (2013). 52. Bertozzi, F.; Bandiera, T. US20190135802A1 (2019). 53. Sasso, O. et al. J Invest Dermatol 138, 562-569 (2018). 54. Pontis, S. et al. Pharmacol Res 160, 105064 (2020). 55. Fiehn, O. et al. Plant J 53, 691-704 (2008). 56. Kind, T. et al. Anal Chem 81, 10038-10048 (2009). 57. Tjolsen, A., Berge, O. G., Hunskaar, S., Rosland, J. H. & Hole, K. Pain 51, 5-17 (1992). 58. Bennett, G. J. & Xie, Y. K. Pain 33, 87-107 (1988). 59. Lueptow, L. M. J Vis Exp (2017). 60. Gaetani, S., Oveisi, F. & Piomelli, D. Neuropsychopharmacology 28, 1311-1316 (2003). 61. Andersson, J. L. R. & Sotiropoulos, S. N. Neuroimage 125, 1063-1078 (2016). 62. Richards, K. et al. Neuroimage 58, 732-740 (2011). 63. Harrison, M. et al. Neuroimage 68, 22-29 (2013). 64. Watson C. et al. Elsevier Academic Press (First edition), 308-312 (2009). 65. Rovini, A. I. Cell Mol Life Sci. 2020 September; 77(18):3691-3692. doi: 10.1007/s00018-019-03417-6. 66. Magalon, K. et al. Neuropharmacology 2016 December; 111:293-303. doi: 10.1016/j.neuropharm.2016.09.009. Epub 2016 Sep. 13. 67. Olesoxime (TRO19622): Pharmaceuticals (Basel). 2010 Jan. 28; 3(2):345-368. doi: 10.3390/ph3020345. 68. Tuo, W. et al. J Med Chem. 2017 Jan. 12; 60(1):4-46. doi: 10.1021/acs.jmedchem.6b00538. Epub 2016 Nov. 14. 69. Ferri, N. et al. Expert Opin Investig Drugs. 2017 May; 26(5):593-602. doi: 10.1080/13543784.2017.1312339. Epub 2017 Apr. 11. 70. Piomelli, D. et al. J Med Chem. 2020 Jul. 23; 63(14): 7475-7490. doi: 10.1021/acs.jmedchem.0c00191. Epub 2020 Mar. 26.

### Example 4

Acute pain after physical trauma can evolve, in vulnerable individuals, into a chronic pain state that long outlasts tissue healing and is often resistant to therapy (1, 2). Nerve damage is considered a critical driving factor in this transition (3, 4). Delineating the nature, localization and timing of such events is necessary to uncover control nodes in the pain chronification process which could be targeted by disease-modifying medicines (5).

Current research efforts are focused on synaptic (3) and immune (4, 6) adaptations that occur, both peripherally and centrally, following end-organ damage though deficits in neuronal energy balance are also receiving serious consideration (7, 8).

The intracellular cysteine hydrolase NAAA (9, 10) catalyzes the degradation of PEA, an endogenous lipid agonist of the nuclear receptor PPAR-α (11). This ligand-operated transcription factor serves important regulatory functions in both cellular metabolism and the defensive response to noxious stimuli (12). Consistent with this dual role, activation of PPAR-α by exogenous PEA attenuates pain (11, 13) and inflammation (14) and stimulates mitochondrial respiration (15). These findings prompted us to ask whether NAAA-regulated PEA signaling at PPAR-α might contribute to the emergence of chronic pain after somatic injury, when the pressure to bring about adaptive neuroplastic changes intensifies the demand for bioenergy in peripheral and central nociceptive neurons (3, 16). Our results show that end-organ damage produces a transient enhancement of NAAA expression and a consequent suppression of PEA-mediated signaling in innervating segments of the spinal cord. This event redirects local metabolism from high energy-yielding mitochondrial respiration toward biomass-generating aerobic glycolysis, a phenomenon akin to the Warburg effect first described in proliferating cancer cells (17). Disabling NAAA during this critical time window restores normal PEA-mediated PPAR-α activation, stops metabolic reprogramming and aborts the transition to pain chronicity. This action identifies NAAA and its cognate signaling complex as a druggable molecular target for the prevention of chronic pain.

Chemical end-organ damage causes a chronic pain-like state in vulnerable mice. To investigate the role of NAAA in the transition to pain chronicity, we adapted the formalin model (18) to allow evaluation of both immediate responses to end-organ lesion and progression to a persistent pain-like state (19, 20). The latter was characterized by assessing key signs of severe chronic pain in humans, including spontaneous pain, bilateral and extraterritorial spreading of sensitization (21, 22), disturbances in emotional, cognitive and vegetative function (23, 24) and microstructural reorganization of forebrain circuits involved in the regulation of stress and emotion (25, 26). We injected escalating doses of formalin (0.1, 0.3 or 1% vol, 20 μL) into the hind paw of male mice and monitored them for the subsequent four months. As expected from prior work (18), the irritant evoked an immediate nocifensive reaction that at the 1% dose lasted >60 min and was associated with robust local inflammation. The pain behavior elicited by 1% formalin, but not 0.1 or 0.3%, was followed in ˜80% of the animals (67/84) by lasting bilateral hypersensitivity to mildly noxious heat stimuli and normally innocuous mechanical stimuli, which appeared within 24-48 h (FIG. 1A) and continued unabated until monitoring was ended (FIG. 1B). Persistent bilateral hypersensitivity was observed in 100% of female mice (22/22) that received formalin (1%). The hypersensitive state (i) emerged independently of the intensity of the initial nocifensive response or the ensuing inflammatory reaction, which were comparable across all animals (FIGS. 1C-1D); (ii) outlasted the resolution of inflammation, which was complete in 60% of male mice by post-formalin day (PFD) 60 (FIG. 1D) and in 100% of male mice by PFD120; (iii) was accompanied by lasting spontaneous/ongoing pain, as shown by assessing the ability of the anti-convulsant gabapentin to elicit conditioned place preference (FIG. 1F); and (iv) was only temporarily alleviated by gabapentin and morphine (FIGS. 1G-1H). Finally, there was strict segregation between mice that developed sensory abnormalities and those that did not, with minimal exchange occurring between the two groups over a 3-week period (FIG. 1I).

Confirming the emergence of bilateral sensitization, we found that administration of a half-maximal dose of the transient receptor potential cation channel subfamily V member 1 (TRPV1) agonist capsaicin (0.1 μg, FIG. 2A) into the uninjured (contralateral) hind paw evoked stronger nocifensive behavior and bilateral spinal cord Fos protein expression in animals vulnerable to the lasting effects of formalin, compared to those that were either resilient to such effects or had received only vehicle (FIGS. 2B-2D). Furthermore, two sets of results were suggestive of trans-segmental spreading of sensitization. First, capsaicin injections into the forepaw produced heightened nociception in mice that were previously given formalin in the contralateral hind paw (FIGS. 2E-2F). Second, ex vivo diffusion tensor imaging (DTI) experiments revealed substantial alterations in directional water diffusivity—an index of microstructural integrity (27) and neural plasticity (28)—throughout the spinal cord of formalin-exposed mice. Most noteworthy, all animals treated with the irritant exhibited lower fractional anisotropy in the lumbar spinal cord but only those that became hypersensitive displayed higher fractional anisotropy in cord segments innervating the forelimbs (FIG. 2G).

In persons living with chronic pain, sensory abnormalities are often associated with emotional, cognitive and vegetative disturbances as well as with morphological reorganization of the limbic forebrain (26). Similarly, in mice vulnerable to the enduring effects of formalin, thermal and mechanical hypersensitivity was accompanied by heightened anxiety-like behavior (FIG. 3A), defective long-term memory (FIG. 3B) and flattened body-gain trajectory (FIG. 3C). There were also conspicuous alterations in forebrain microstructure. FIGS. 3D-3E illustrate the appearance of bilateral differences in the infralimbic prefrontal cortex and tenia tecta, two grey matter regions that participate in the control of pain (29), memory (30, 31) and stress (32, 33) (FIGS. 3D-3E). Moreover, a unilateral volume increase was statistically detectable in the right hippocampal CA2 subfield, which is involved in social memory (34), while a trend toward increase (P=0.06) was noted in the right basolateral amygdala, whose role in the emotional dimension of pain is well recognized (35) (FIG. 3D). The finding that these alterations were not detectable in resilient formalin-injected mice (FIGS. 3F-3G) supports the possibility that they might contribute to the enduring pain-like phenotype exhibited by vulnerable animals. In addition to changes in grey matter, bilateral volume differences between formalin-exposed (both vulnerable and resilient) and control mice were observed in various fiber tracts, including the anterior commissure, dorsal fornix and dorsal hippocampal fissure (FIG. 3E). The results show that formalin-induced end-organ damage triggers in susceptible mice the development of a long-lasting neuropathological state whose multimodal manifestations are strikingly reminiscent of severe chronic pain in humans. For brevity, we will refer to this condition as ‘chronic pain-like state’ (CPLS).

NAAA controls the transition to pain chronicity. We used a genetic loss-of-function/gain-of-function strategy as a first step toward assessing whether NAAA might contribute to the emergence of formalin-induced CPLS. Mice constitutively lacking the enzyme (36) displayed normal nociceptive thresholds, motor activity and feeding patterns. Nevertheless, formalin (1%) elicited in these mutants a weakened and shorter spontaneous nocifensive response, compared to their wild-type littermates, and did not cause inflammation or persistent hypersensitivity (FIGS. 4A-4C). Raising the formalin concentration to 3% increased nociceptive responding and evoked pronounced edema and ipsilateral hypersensitivity in NAAA-null mice but still failed to produce lasting contralateral sensitization (FIGS. 4D-4F). This phenotype was recapitulated in wild-type mice by pretreatment with either of two reversible and highly selective NAAA inhibitors, ARN19702 (30 mg-kg⁻¹, IP) and ARN16186 (10 mg-kg⁻¹, IP) (10) (FIGS. 4G-4I). Neither compound affected baseline nociceptive thresholds in naïve animals. Conversely, mice overexpressing NAAA in CD11b-positive cells (i.e., monocytes, macrophages and microglia), which play important roles in pain chronification (3, 4), displayed robust nociception, inflammation and persistent hypersensitivity when challenged with a formalin dose (0.1%) that had negligible impact on control animals (FIGS. 4J-4L). The findings suggest that NAAA facilitates acute defensive reactions to injury and point to a possible role for the enzyme in the development of formalin-induced CPLS.

To delineate such a role, we administered ARN19702 (30 mg-kg⁻¹, IP) or its vehicle to male mice once daily for 3 consecutive days starting 24 h after the formalin challenge, and evaluated sensory, cognitive, emotional, vegetative and microstructural outcomes during the following two weeks. Despite its short duration, treatment with the NAAA inhibitor effectively halted CPLS consolidation, normalizing evoked hypersensitivity and ongoing pain, anxiety-like behavior, memory deficits and body-weight gain (FIGS. 5A-5E) and reversing CPLS-associated changes in spinal and forebrain DTI measures (FIGS. 5F-5G). Furthermore, the protective effects of ARN19702 were mimicked by a second NAAA inhibitor, ARN16186 (10 mg-kg⁻¹, IP) (FIG. 5A), and were not sex-dependent (FIG. 5H).

Unlike agents that interfere with NAAA activity, maximally effective dosages of four mechanistically distinct analgesic and anti-inflammatory drugs—gabapentin (50 mg-kg⁻¹, IP), morphine (10 mg-kg⁻¹, subcutaneous), ketamine (4 mg-kg⁻¹, IP) and ketoprofen (100 mg-kg⁻¹, IP)—failed to stop the conversion to CPLS when administered on PFD2-4 (FIGS. 5I-5L). Additionally, even though NAAA blockade attenuates inflammation in rodent models (9), the three-day ARN19702 regimen had no detectable influence on paw edema (FIG. 5M), likely owing to its brief duration. We interpret these findings as indicating that NAAA inhibitors halt the transition to pain chronicity through a mechanism that is distinguishable from their antinociceptive and anti-inflammatory properties. To mark this distinction, we will refer to such mechanism as ‘algostatic’—from the ancient Greek \check{α}λγoç ‘pain’ and iστ\acute{α}vαι ‘to stop’.

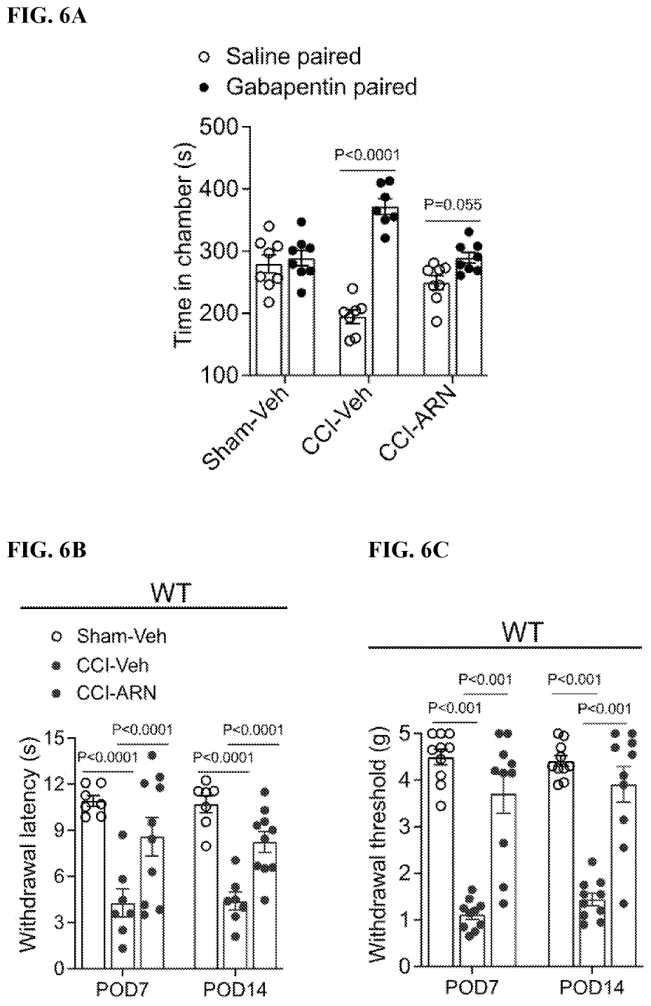

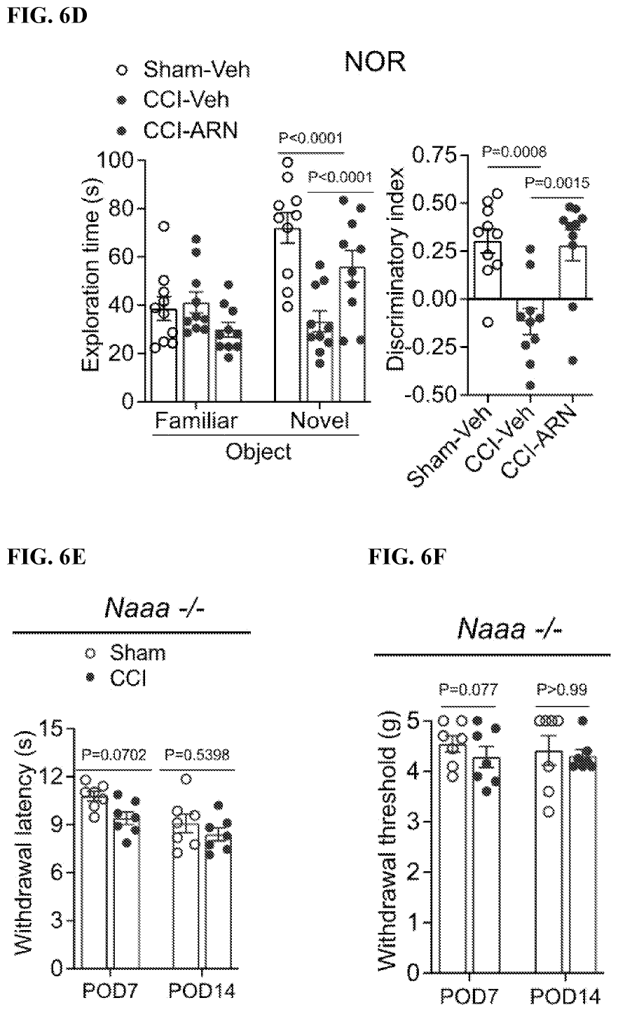

To determine whether the algostatic effects of NAAA inhibition could be generalized to other models of persistent pain, we asked whether ARN19702 might affect the development of hypersensitivity, spontaneous pain and cognitive abnormalities in mice subjected to chronic constriction injury (CCI) of the sciatic nerve (37). We loosely tied the nerve under anesthesia and, starting 24 h later, administered ARN19702 (30 mg-kg⁻¹, IP) or its vehicle once daily for 3 days. Spontaneous pain and hypersensitivity to heat and mechanical stimuli were assessed on post-operative days (POD) 7-14, and long-term memory was assessed on POD 26. Treatment with the NAAA inhibitor prevented the appearance of all chronic pain manifestations (FIGS. 6A-6D). As expected from prior results (FIGS. 4A-4F), genetic NAAA deletion also blocked the development of thermal and mechanical hypersensitivity (FIGS. 6E-6F). We conclude that post-injury NAAA removal interrupts the emergence of chronic pain-like states elicited in mice by either chemical or mechanical tissue damage.

A critical period for pain chronification. The ability of NAAA inhibitors to stop CPLS consolidation was crucially dependent on the timing of their administration. This was demonstrated by the experiment illustrated in FIGS. 8A-8B, in which a single injection of ARN19702 (30 mg-kg⁻¹, IP) was administered to separate groups of male wild-type mice on PFD1, 5, 6, 7, 8 or 9. The NAAA inhibitor provided full protection when given once on PFD3 or PFD4 but had no such effect when given on PFD1 or PFD5 to PFD9 (FIG. 8B)—even though at all time points it temporarily attenuated pain behaviors. Administration on PFD2 was partially effective. The time window for NAAA-inhibitor efficacy coincided with a transient rise in Naaa gene transcription in the lumbar (L4-L6) spinal hemicord ipsilateral to the injured paw, which was maximal at PFD3 (FIG. 8C, left). No change occurred on the contralateral side, where Naaa mRNA content remained stable from PFD1 to PFD5 (FIG. 8C, right). The spike in Naaa transcription was associated with higher levels of immunoreactive NAAA (FIGS. 8D-8G) and with a reduction in local PEA levels (FIG. 8H). Co-localization studies showed that immunoreactive NAAA was primarily localized to NeuN-positive cells (neurons) of the dorsal and ventral horns (FIGS. 8E-8G), though sparse Olig2-positive cells (oligodendrocytes) were also detected. Importantly, accrued spinal cord NAAA activity was required for the induction of persistent hypersensitivity elicited by formalin or CCI, as this was stopped by post-injury intrathecal infusion of either ARN19702 or ARN077, a covalent NAAA inhibitor that does not cross the blood-brain barrier (9) (FIGS. 81-8J). In sum, the results show that end-organ damage sets off a transitory suppression of NAAA-regulated PEA signaling in innervating segments of the spinal cord, which starts 24 h-48 h after the injury, lasts approximately 72 h, and is required for pain to become chronic.

Transcriptomic and metabolomic experiments offer important insights into the molecular events unfolding during this critical period. RNA sequencing analyses of L4-L6 spinal cord fragments ipsilateral to the formalin injection site identified large-scale transcriptional changes at PFD3 and PFD4, compared to time- and site-matched controls. At PDF4, when changes were largest, differentially upregulated transcripts were significantly enriched in the following Gene Ontology categories: synaptic membrane [adjusted P value (P_{adj})=1.17⁻³⁵], neuron to neuron synapse (P_{adj}=7.82⁻³⁰), postsynaptic membrane (P_{adj}=8.42⁻³⁰), glutamatergic synapse (P_{adj}=8.40⁻²⁷) and axon development (P_{adj}=2.53⁻²⁶). Notably, expression of neuronal genes encoding for, among others, voltage-gated sodium channels (e.g., Scn1a and Scn8a), calcium channels (e.g., Cacn1a and Cacn1b) and α-amino-3-hydroxy-5-methyl-4-isoxazole propionic acid (AMPA) receptor-regulating proteins (e.g., Cacng2 and Cacng5) was strongly enhanced. Transcription of key components of cholesterol biosynthesis (e.g., Hmgcr, Hmges2)—a requisite for membrane biogenesis (38, 39)—was also heightened. These changes are consistent with evidence of widespread synaptic (16) and proteomic (40) modifications in spinal cord of animals experiencing chronic pain states. By contrast, differentially downregulated transcripts at PFD4 were enriched in categories including mitochondrial protein complex (P_{adj}=3.93⁻⁶⁹), inner mitochondrial membrane protein complex (P_{adj}=4.03⁻⁴⁷) and mitochondrial respiratory chain (P_{adj}=2.01⁻³²) (FIG. 8b). Accordingly, many protein components of tricarboxylic acid (TCA) cycle and oxidative phosphorylation were suppressed on PFD4 (FIG. 8d). The opposite occurred, however, with two members of the bifunctional 6-phosphofructo-2-kinase fructose-2,6-biphosphatase (Pfkfb) family (Pfkfb3 and Pfkfb4) and with the glucose transporter Glut1 (Slc2a12). Of note, Pfkfb3 shunts glucose toward glycolysis whereas Pfkfb4 redirects glucose toward the pentose phosphate pathway, suggesting that both processes might be activated during the critical period for CPLS development (41).

The transcriptional shift was paralleled by a largely concordant set of metabolomic changes. Decreased concentrations of free amino acids and non-esterified unsaturated fatty acids together with increased cholesterol and phospholipid precursors (zymosterol and sn-glycerol-3-phosphate, respectively) support the possibility that protein synthesis and membrane biogenesis are accelerated on PFD4. Concomitantly, N-acetyl-L-aspartate, which provides carbon units for neural lipid biosynthesis (42), was increased (FIG. 8G) whereas urea cycle intermediates ornithine and citrulline declined, possibly reflecting accrued nitrogen recycling (43). Furthermore, elevated glucose, glucose-1-phosphate (G1P), glucose-6-phosphate (G6P), fructose-6-phosphate (F6P) and ribulose-5-phosphate (Ru5P) at PFD3 and/or PFD4 were suggestive of upregulated glucose transport, glycogenolysis (G1P), glycolysis (G6P, F6P) and the oxidative branch of the pentose phosphate pathway (Ru5P). The small but significant decrease in citric acid on PFD3 was consistent with reduced TCA cycle activity, while unchanged lactate levels suggested that glycolysis-derived pyruvate (which was undetectable in our samples) may have been utilized for biosynthetic needs. Together, the above alterations may account for the rise in 5′-adenosine monophosphate (AMP) and 5′-inosine monophosphate (IMP), two nucleotides that accumulate in cells during energy crises (44). Lastly, the neurotransmitters glutamate and 5-hydroxytryptamine were increased whereas γ-aminobutyric acid was decreased, possibly due to imbalanced excitation/inhibition and enhanced descending serotonergic input (16). Thus, the critical period for the consolidation of formalin-induced CPLS coincides with a transcriptionally controlled metabolic switch from mitochondrial respiration to aerobic glycolysis, which is localized to spinal cord segments that receive direct nociceptive input from the lesioned paw. Systemic ARN19702 administration on PFD2-3 prevented the appearance of transcriptional and metabolic changes on the following day, demonstrating a critical role for NAAA in enabling this switch.

NAAA governs pain chronification via PEA signaling at PPAR-α. As noted above, PEA levels in ipsilateral lumbar spinal cord (L4-L6) of formalin-injected mice were markedly reduced on PFD4, compared to vehicle-treated controls (FIG. 9A). Post-formalin ARN19702 administration normalized such levels (FIG. 9A), suggesting that PPAR-α—which is expressed in neurons (11, 45) and other cells of the central nervous system (45)—might mediate the algostatic effects of NAAA inhibition. Experiments with mice constitutively lacking the nuclear receptor confirmed this possibility. When challenged with either 0.1% or 1% formalin, PPAR-α-null mice exhibited substantially stronger nociception compared to wild-type controls (FIG. 9B, FIG. 9E), which gave way at both doses to inflammation (FIG. 9c, f) and lasting hypersensitivity (FIG. 9D, FIG. 9G). Importantly, in these mutants the transition to CPLS was unaffected by ARN19702 (30 mg-kg⁻¹, IP) (FIG. 9H), a trait that was replicated in wild-type animals by pretreatment with the selective PPAR-α antagonist GW6471 (4 mg-kg⁻¹, IP) (FIG. 9I). Further supporting a pivotal role for PPAR-α in gating pain chronification, we found that post-injury administration of the highly potent and selective PPAR-α agonist GW7647 (10 mg-kg⁻¹, IP) stopped this process in three different experimental settings: (i) wild-type mice treated with 1% formalin (FIG. 9J); (ii) NAAA-overexpressing mice treated with 0.1% formalin (FIG. 9K); and (iii) wild-type mice subjected to CCI (FIG. 9L). Like NAAA inhibition, PPAR-α activation by GW7647 prevented injury-associated changes in spinal-cord transcription. Finally, CPLS was blocked by exogenous PEA (30 mg-kg⁻¹, subcutaneous) but not by the weak PPAR-α agonist fenofibrate (100 mg-kg⁻¹, IP) (FIGS. 9M-9N), which is used in the clinic to treat hyperlipidemia.

Twenty percent of the adult world population—approximately 1.5 billion people—suffer from chronic pain (46, 47), yet its management continues to depend on a handful of analgesic drug classes, such as the opioids, which have dubious long-term effectiveness and can cause addiction (48). In fact, epidemiological studies indicate that chronic pain is a major risk factor for opioid use disorder, with more than 60% of people who misused the drugs reporting to have done so to achieve pain relief (49). The development of chronic pain is often associated with accidental or surgical tissue trauma. For example, a review of five independent surveys found that more than half of patients who underwent thoracotomy reported experiencing pain one year after surgery, while 21% were still in pain seven years later (1). Nerve damage may contribute to pain chronification after physical trauma, but the sequence of events underlying this process—including its molecular and cellular components, timing and anatomical localization—remains largely unknown (1, 5). In the present report, we show that NAAA-regulated PEA signaling at PPAR-α is a critical control point in the transition to chronic pain after tissue injury, which can be effectively targeted by small-molecule therapeutics. Using two distinct experimental models, one of which is fully characterized here, we found that disabling intracellular NAAA activity in spinal cord during a brief critical period after end-organ damage forestalls the emergence of chronic pain by reprogramming local metabolism from aerobic glycolysis to mitochondrial respiration. The results identify NAAA and its cognate signaling complex as a molecular target for the prevention of chronic pain consolidation following a traumatic injury.

As we set out to examine NAAA's possible roles in the progression to pain chronicity, we selected two models—intraplantar formalin injection and CCI of the sciatic nerve—that would allow us to capture this transition. In the formalin test, which was originally developed to investigate acute nociceptive responses to tissue injury (18), subcutaneous injection of this irritant produces a nocifensive reaction that lasts approximately 60 min and is followed by localized inflammation and persistent hypersensitivity to heat and pressure (19, 20, 50). Our current studies indicate that this lasting state (abbreviated here as CPLS) closely retraces key manifestations of severe chronic pain in humans, including spontaneous pain, bilateral and trans-segmental spreading of sensitization (21, 22, 51), disturbances in emotional, cognitive and vegetative function (23, 24, 52, 53) and reorganization of forebrain areas involved in the control of stress and emotion (e.g., prefrontal cortex and basolateral amygdala) (25, 26, 54). While some of these abnormalities have been described in neuropathic pain models such as CCI (e.g., increased anxiety-like behaviors, memory deficits) (55), others have not (e.g., extraterritorial spreading of sensitization). Moreover, our results show that CPLS emerges independently of the inflammatory reaction to formalin (FIGS. 1C-1E) and outlasts the resolution of inflammation (FIG. 1E), ostensibly aligning the tail-end of this model with human pathologies in which pain is the sole complaint (56). Despite these useful features, which warrant further evaluation, formalin-induced CPLS has no face validity as a model to study persistent pain produced by accidental or surgical trauma, a common root cause of this condition (1). For this reason, we also used the CCI model, a well-established paradigm in which mechanical damage to the sciatic nerve results in lasting sensory, emotional and cognitive abnormalities (55). Despite their differences, the two models yielded comparable responses to pharmacological or genetic interventions targeting NAAA-regulated PEA signaling.

Preclinical evidence points to NAAA as a potential target for analgesic and anti-inflammatory therapy (10, 57). In peripheral tissues, where the functions of PEA are better understood (58, 59), sensory neurons of the dorsal root ganglia (DRG), tissue-resident macrophages and other host-defense cells generate this lipid mediator in amounts that are sufficient to maintain PPAR-α in an activated state. Following tissue damage, danger signals such as bacterial endotoxin initiate a molecular program that lowers PEA production and heightens NAAA-catalyzed PEA degradation, resulting in an overall reduction in PPAR-α-mediated signaling and a consequent enhancement of the inflammatory response (60). Consistent with this scenario, NAAA inhibitors and PEA display comparable antinociceptive and anti-inflammatory properties in animal models. For example, topical application of the covalent NAAA inhibitor (S)-OOPP reinstates normal PEA levels in activated leukocytes and blunts inflammatory responses induced by carrageenan or endotoxin (60). Similarly, the β-lactone derivative ARN077, which also inhibits NAAA covalently, corrects sensory abnormalities caused in mice and rats by carrageenan injection, ultraviolet B-radiation or CCI (61). These effects are mimicked by exogenous PEA, are absent in PPAR-α-null mice and are prevented, in rats, by the selective PPAR-α antagonist GW6471 (61). Furthermore, ARN077 reduces mechanical hypersensitivity in fibrosarcoma-bearing mice and rapidly normalizes calcium signaling in DRG neurons co-cultured with fibrosarcoma cells (62). Confirming its proposed mechanism of action, ARN077 restores baseline PEA levels in diseased tissues of CCI (61) and tumor-bearing mice (62). Other chemically and mechanistically different NAAA inhibitors exert similar effects (10) For example, the non-covalent agent ARN19702 suppresses nociceptive behavior evoked by formalin or CCI as effectively as does ARN077, which acts via covalent modification (63). Indeed, a meta-analysis commissioned by the International Association for the Study of Pain recently noted that NAAA inhibitors “produced the largest significant attenuation of pain-associated behavior compared to control” among all endocannabinoid- and cannabinoid-related agents reported in the preclinical literature (57). Though PEA-dependent recruitment of PPAR-α is required for the rapid antinociceptive properties of NAAA inhibitors, the mechanism through which activated PPAR-α modulates nociception is still unclear. Pharmacological studies have implicated the opening of calcium-operated potassium channels (11), but the existence of other mechanisms cannot be ruled out.

The present report shows that, in addition to participating in the control of nociception, NAAA-regulated PEA signaling at PPAR-α also governs the progression to pain chronicity by serving, at a critical juncture of the pain consolidation process, as a regulatory checkpoint for spinal cord metabolism. This hypothesis is consistent with the established roles of PPAR-α in the transcriptional control of lipid and glucose utilization (12) and is supported by three lines of evidence. First, formalin injection into the mouse hind paw causes a short-lived rise in NAAA expression in ipsilateral L4-L6 spinal cord, which reaches its maximum 72 h after the injury (FIG. 8C). This coincides with a transcriptionally regulated switch in cellular bioenergy production from mitochondrial respiration to aerobic glycolysis and pentose phosphate metabolism (further discussed below). Second, systemic or intrathecal administration of one of three different NAAA inhibitors at the peak of NAAA expression (i.e., 48 h-72 h after the injury) shuts off this metabolic switch and simultaneously stops the transition to CPLS (FIG. 8I). Third, mutant mice lacking Naaa do not progress to CPLS even when they are challenged with a suprathreshold dose of formalin (3%) that evokes substantial nocifensive behavior, local inflammation and ipsilateral hypersensitivity (FIGS. 4D-4F). These disease-modifying properties of NAAA inhibitors can be readily distinguished from those of other therapeutic drug classes. Indeed, we found that four analgesic and anti-inflammatory agents used in the clinic—morphine, gabapentin, ketamine and ketoprofen—fail to prevent CPLS when administered at the peak of NAAA-inhibitor efficacy. On the other hand, administration of a NAAA inhibitor during this critical period stops CPLS development but does not affect the size of the inflammatory response or the time course of its resolution. Furthermore, NAAA inhibitors do not visibly accelerate tissue healing, as seen, for example, with agents that block fatty acid amide hydrolase activity (64). To mark the ability of NAAA inhibitors and PPAR-α agonists to halt the progression to pain chronicity—which differentiates them from analgesic, anti-inflammatory, pro-resolving and pro-reparative agents—we used the neologism ‘algostatic’, from Ancient Greek \check{α}λγoç ‘pain’ and στ\acute{α}ναι ‘to stop’.

The role of cellular energy metabolism in the transition to chronic pain after tissue injury deserves consideration. End-organ damage poses an extraordinary bioenergetic challenge to first- and second-order nociceptors, as it pressures them to enact large-scale neuroplastic adaptations while simultaneously maintaining energy homeostasis along the considerable distance of their axonal trees (>1 m in humans) (65). Finite neuronal resources must be allocated to two equally urgent tasks: heightened energy production to sustain the upsurge in neural activity caused by the lesion, and biomass generation to effect the structural changes needed to support peripheral and central sensitization (16). The results of our transcriptomic and metabolomic experiments suggest that mouse spinal cord neurons negotiate this conflict—most likely in concert with cells of other lineages (see further discussion below)—by temporarily shifting ATP production from mitochondrial respiration, which is energetically efficient but has substantial proteomic costs (66), to Warburg-like aerobic glycolysis (17), which is far less efficient but can generate carbon units for protein and lipid biosynthesis. A maladaptive consequence of this metabolic switch, whose occurrence in differentiating (67) and acutely stimulated adult neurons (68, 69) is documented, is the instigation of an energy crisis which might disrupt electrochemical gradients and ultimately precipitate central sensitization. An interesting parallel of this hypothetical scenario is offered by macrophages, which shift their metabolism toward glycolysis when transitioning to trained immunity (70), a sensitized state that heightens both their responsiveness to recurring infections and the risk of unleashing a hyperinflammatory reaction (71). This phenotype shift is stopped by the anti-diabetic drug metformin (71), which also prevents neuropathic pain in male (but not female) rodents (72). In both instances, metformin may work by indirectly recruiting AMP-activated protein kinase, which reacts to cellular energy crises by stimulating ATP generation and suppressing anabolic processes (73). Despite their obvious dissimilarities, these two responses to noxious signals appear to rely on a shared proteome allocation strategy that transiently favors aerobic glycolysis over oxidative phosphorylation—a suboptimal trade-off (49) that may render affected cells persistently hypersensitive to an otherwise innocuous stimulus.

The present results raise three important questions. The first pertains to the identity of the cellular elements involved in the algostatic effects of NAAA inhibition. Spinal cord neurons constitutively express NAAA and may be directly impacted by NAAA blockade. But the enzyme (or the mRNA encoding for it) is also found in spinal astrocytes and microglia [(74), http://neuroexpresso.org] as well as in circulating monocytes and resident tissue macrophages (9), whose roles in the pathogenesis and resolution of chronic pain are well recognized (75, 76). Our finding that NAAA overexpression in CD11b-positive cells (i.e., monocytes, macrophages and microglia) facilitates the induction of CPLS (FIGS. 4J-4L) suggests that at least one of these cell types may be targeted by NAAA inhibitors. The second question relates to the role that NAAA might play in the resilient phenotype exhibited by 20% of male (not female) mice which exhibit normal acute responses to formalin but fail to transition to CPLS (FIGS. 1B-1C). In humans, resilience toward developing chronic pain is sexually dimorphic (women are on average less resilient) (47, 77) and depends on multiple preexisting factors that include age, genetic make-up and disease history (2). Untangling NAAA's role in this complex scenario is an important topic for future research. The last question pertains to the mechanism through which nerve injury increases NAAA expression in spinal cord neurons. Given the paucity of information regarding NAAA's transcriptional control (10), a variety of scenarios appear equally plausible. One intriguing possibility is that release of colony stimulating factor-1 (78) or other cytokines (e.g., CXCL1) (79) in the dorsal horn might directly or indirectly stimulate NAAA transcription. In this regard it is important to point out that NAAA may also be activated through a posttranslational mechanism involving the autocatalyzed cleavage of its inactive precursor (10).

The above questions notwithstanding, the present results identify a previously unrecognized control node in the transition from acute to chronic pain, which can be targeted by NAAA inhibitors or by other agents that enhance PEA-mediated signaling at PPAR-α. Owing to their distinctive pharmacological profile—which combines antinociception, anti-inflammation and algostasis—NAAA inhibitors might find clinical application in the treatment of various forms of chronic pain as well as in perioperative strategies aimed at preventing the establishment of central sensitization caused by incisional and inflammatory injuries (80).

### Methods

Chronic constriction injury (CCI) of the sciatic nerve was carried out as described (37). Briefly, the mice were anesthetized with isoflurane and the right common sciatic nerve was exposed at the level of the middle thigh by blunt dissection under aseptic conditions. Proximal to the trifurcation, the nerve was cleaned from surrounding connective tissue and 3 chromic cat gut ligatures (4-0, Ethicon, Somerville, USA) were loosely tied around it at 1-mm intervals. The wound was closed with a single muscle suture and skin clips. In sham-operated animals, the nerve was exposed but not tied.

Sensitivity to heat was measured using a Hargreaves plantar test apparatus (San Diego Instruments, San Diego, USA). After a 45-min habituation period, the plantar surface of both hind paws was exposed to a beam of radiant heat through the glass floor. The cut-off time was set at 15 s. The stimulation was repeated 3 times with an interval of 2 min between stimuli and latencies (in s) to withdraw the paw were recorded and averaged.

Elevated plus maze test: We placed each mouse in the central platform of the maze, facing the open arm opposite to the experimenter, and behavior was recorded using the Debut video capture software (NCH Software, Canberra, Australia). A blinded observer measured the amount of time spent in the open and closed arms, as well as the number of open and closed arm entries. The open arms of the maze were illuminated at 150-170 lux and the closed arms at 40-50 lux. The anxiety index was calculated as follows:

$$Anxiety index = 1 - \frac{( \frac{time in open arms}{total time} ) + ( \frac{n umber of open arms entries}{total entries} )}{2}$$

Novel Object Recognition test was performed as described (59). The test was conducted over 3 days. On day 1, mice were habituated to the empty arena for 20 min. On day 2, they were returned to the arena, which now contained 2 identical objects. On day 3, one of the objects was replaced with another one of different shape, color and texture. Mice were allowed to explore the arena for 10 min, and the total time spent exploring each object (i.e., nosing and sniffing at a distance ≤2 cm) was recorded by a blinded observer. The discriminatory index was calculated as follows:

$$Discriminatory = \frac{( time of novel object exploration ) - ( time of familiar object exploration )}{total exploration time}$$

Conditioned Placed Preference: To determine whether end-organ damage resulted in long-lasting ongoing or spontaneous pain (85), mice were subjected to either formalin (1%) injection or CCI injury as described above and a single-trial CPP test was performed 14 days later. The test consisted of three phases: preconditioning, conditioning and preference assessment.

Preconditioning: 12 days after tissue damage, mice were placed in the two chamber CPP apparatus (for details see (63)) and the time spent in each chamber was recorded for 10 min and analyzed to determine preconditioning baseline. Individual mice that showed more than 80% of preference during preconditioning were excluded.

Conditioning: Mice underwent single trial conditioning 24 h after preconditioning using a biased approach. In the morning of the conditioning day, animals received saline and were immediately placed into the preferred chamber (saline paired) of the CPP box for 30 min, with no access to the other chambers. Approximately 4 h later, mice received gabapentin (50 mg-kg⁻¹, IP) and were immediately confined in the non-preferred chamber for 30 min (gabapentin paired).

Preference test: On the test day (POD14 or PFD14), 12 h after the afternoon pairing, mice were placed back into the center of the CPP chambers with free access to all chambers for 10 min. Time (s) spent in each chamber was recorded and evaluated by a blind observer to determine preference.

### REFERENCES FOR EXAMPLE 4

1. C. R. Chapman, C. J. Vierck, J Pain 18, 359 e351-359 e338 (2017). 2. F. Denk, S. B. McMahon, I. Tracey, Nat Neurosci 17, 192-200 (2014). 3. A. I. Basbaum, D. M. Bautista, G. Scherrer, D. Julius, Cell 139, 267-284 (2009). 4. R. R. Ji, A. Chamessian, Y. Q. Zhang, Science 354, 572-577 (2016). 5. T. J. Price, A. I. Basbaum, J. Bresnahan, J. F. Chambers, Y. De Koninck, R. R. Edwards, R. R. Ji, J. Katz, A. Kavelaars, J. D. Levine, L. Porter, N. Schechter, K. A. Sluka, G. W. Terman, T. D. Wager, T. L. Yaksh, R. H. Dworkin, Nat Rev Neurosci 19, 383-384 (2018). 6. F. Marchand, M. Perretti, S. B. McMahon, Nat Rev Neurosci 6, 521-532 (2005). 7. E. K. Joseph, J. D. Levine, Pain 121, 105-114 (2006). 8. G. J. Bennett, T. Doyle, D. Salvemini, Nat Rev Neurol 10, 326-336 (2014). 9. K. Tsuboi, L. Y. Zhao, Y. Okamoto, N. Araki, M. Ueno, H. Sakamoto, N. Ueda, Biochim Biophys Acta 1771, 623-632 (2007). 10. D. Piomelli, L. Scalvini, Y. Fotio, A. Lodola, G. Spadoni, G. Tarzia, M. Mor, J Med Chem 63, 7475-7490 (2020). 11. J. LoVerme, R. Russo, G. La Rana, J. Fu, J. Farthing, G. Mattace-Raso, R. Meli, A. Hohmann, A. Calignano, D. Piomelli, J Pharmacol Exp Ther 319, 1051-1061 (2006). 12. N. Bougarne, B. Weyers, S. J. Desmet, J. Deckers, D. W. Ray, B. Staels, K. De Bosscher, Endocr Rev 39, 760-802 (2018). 13. B. B. Artukoglu, C. Beyer, A. Zuloff-Shani, E. Brener, M. H. Bloch, Pain Physician 20, 353-362 (2017). 14. L. Rankin, C. J. Fowler, Int J Mol Sci 21, (2020). 15. C. Annunziata, A. Lama, C. Pirozzi, G. Cavaliere, G. Trinchese, F. Di Guida, A. Nitrato Izzo, F. Cimmino, O. Paciello, D. De Biase, E. Murru, S. Banni, A. Calignano, M. P. Mollica, G. Mattace Raso, R. Meli, FASEB J 34, 350-364 (2020). 16. A. Latremoliere, C. J. Woolf, J Pain 10, 895-926 (2009). 17. M. V. Liberti, J. W. Locasale, Trends Biochem Sci 41, 211-218 (2016). 18. D. Dubuisson, S. G. Dennis, Pain 4, 161-174 (1977). 19. L. Luongo, F. Guida, S. Boccella, G. Bellini, L. Gatta, F. Rossi, V. de Novellis, S. Maione, CNS Neurol Disord Drug Targets 12, 45-54 (2013). 20. K. Y. Fu, A. R. Light, G. K. Matsushima, W. Maixner, Brain Res 825, 59-67 (1999). 21. L. Arendt-Nielsen, Handb Exp Pharmacol 227, 79-102 (2015). 22. K. H. Konopka, M. Harbers, A. Houghton, R. Kortekaas, A. van Vliet, W. Timmerman, J. A. den Boer, M. M. Struys, M. van Wijhe, PLOS One 7, e37524 (2012). 23. G. J. Asmundson, J. Katz, Depress Anxiety 26, 888-901 (2009). 24. K. S. Baker, S. Gibson, N. Georgiou-Karistianis, R. M. Roth, M. J. Giummarra, Clin J Pain 32, 673-680 (2016). 25. A. E. Metz, H. J. Yau, M. V. Centeno, A. V. Apkarian, M. Martina, Proc Natl Acad Sci USA 106, 2423-2428 (2009). 26. M. N. Baliki, A. V. Apkarian, Neuron 87, 474-491 (2015). 27. M. Kaushal, S. Shabani, M. Budde, S. Kurpad, J Neurotrauma 36, 2279-2286 (2019). 28. A. A. Schlegel, J. J. Rudelson, P. U. Tse, J Cogn Neurosci 24, 1664-1670 (2012). 29. S. Shiers, G. Pradhan, J. Mwirigi, G. Mejia, A. Ahmad, S. Kroener, T. Price, J Neurosci 38, 7337-7350 (2018). 30. H. Eichenbaum, Nat Rev Neurosci 18, 547-558 (2017). 31. L. B. Haberly, Chem Senses 26, 551-576 (2001). 32. N. Kataoka, Y. Shima, K. Nakajima, K. Nakamura, Science 367, 1105-1112 (2020). 33. M. L. Lehmann, M. Herkenham, J Neurosci 31, 6159-6173 (2011). 34. F. L. Hitti, S. A. Siegelbaum, Nature 508, 88-92 (2014). 35. G. Ji, V. Neugebauer, J Neurophysiol 102, 2253-2264 (2009). 36. O. Sasso, M. Summa, A. Armirotti, S. Pontis, C. De Mei, D. Piomelli, J Invest Dermatol 138, 562-569 (2018). 37. G. J. Bennett, Y. K. Xie, Pain 33, 87-107 (1988). 38. A. Nohturfft, S. C. Zhang, Annu Rev Cell Dev Biol 25, 539-566 (2009). 39. C. J. Stefan, W. S. Trimble, S. Grinstein, G. Drin, K. Reinisch, P. De Camilli, S. Cohen, A. M. Valm, J. Lippincott-Schwartz, T. P. Levine, D. B. Iaea, F. R. Maxfield, C. E. Futter, E. R. Eden, D. Judith, A. R. van Vliet, P. Agostinis, S. A. Tooze, A. Sugiura, H. M. McBride, BMC Biol 15, 102 (2017). 40. D. Gomez-Varela, A. M. Barry, M. Schmidt, J Proteomics 190, 1-11 (2019). 41. M. Yi, Y. Ban, Y. Tan, W. Xiong, G. Li, B. Xiang, Mol Metab 20, 1-13 (2019). 42. J. B. Clark, Dev Neurosci 20, 271-276 (1998). 43. J. B. Spinelli, H. Yoon, A. E. Ringel, S. Jeanfavre, C. B. Clish, M. C. Haigis, Science 358, 941-946 (2017). 44. C. R. Hancock, J. J. Brault, R. L. Terjung, J Physiol Pharmacol 57 Suppl 10, 17-29 (2006). 45. A. Warden, J. Truitt, M. Merriman, O. Ponomareva, K. Jameson, L. B. Ferguson, R. D. Mayfield, R. A. Harris, Sci Rep 6, 27618 (2016). 46. D. S. Goldberg, S. J. McGee, BMC Public Health 11, 770 (2011). 47. J. Dahlhamer, J. Lucas, C. Zelaya, R. Nahin, S. Mackey, L. DeBar, R. Kerns, M. Von Korff, L. Porter, C. Helmick, MMWR Morb Mortal Wkly Rep 67, 1001-1006 (2018). 48. H. Reinecke, C. Weber, K. Lange, M. Simon, C. Stein, H. Sorgatz, Br J Pharmacol 172, 324-333 (2015). 49. B. Han, W. M. Compton, C. Blanco, E. Crane, J. Lee, C. M. Jones, Ann Intern Med 167, 293-301 (2017). 50. J. H. Rosland, A. Tjolsen, B. Maehle, K. Hole, Pain 42, 235-242 (1990). 51. C. P. van Wilgen, K. H. Konopka, D. Keizer, J. Zwerver, R. Dekker, Scand J Med Sci Sports 23, 149-155 (2013). 52. M. J. Bair, R. L. Robinson, W. Katon, K. Kroenke, Arch Intern Med 163, 2433-2445 (2003). 53. G. M. Grace, W. R. Nielson, M. Hopkins, M. A. Berg, J Clin Exp Neuropsychol 21, 477-487 (1999). 54. L. Goncalves, R. Silva, F. Pinto-Ribeiro, J. M. Pego, J. M. Bessa, A. Pertovaara, N. Sousa, A. Almeida, Exp Neurol 213, 48-56 (2008). 55. M. G. Liu, J. Chen, Prog Neurobiol 116, 13-32 (2014). 56. R. D. Treede, W. Rief, A. Barke, Q. Aziz, M. I. Bennett, R. Benoliel, M. Cohen, S. Evers, N. B. Finnerup, M. B. First, M. A. Giamberardino, S. Kaasa, B. Korwisi, E. Kosek, P. Lavand′homme, M. Nicholas, S. Perrot, J. Scholz, S. Schug, B. H. Smith, P. Svensson, J. W. S. Vlaeyen, S. J. Wang, Pain 160, 19-27 (2019). 57. N. Soliman, S. Haroutounian, A. G. Hohmann, E. Krane, J. Liao, M. Macleod, D. Segelcke, C. Sena, J. Thomas, J. Vollert, K. Wever, H. Alaverdyan, A. Barakat, T. Barthlow, A. L. H. Bozer, A. Davidson, M. Diaz-delCastillo, A. Dolgorukova, M. I. Ferdousi, C. Healy, S. Hong, M. Hopkins, A. James, H. B. Leake, N. M. Malewicz, M. Mansfield, A. K. Mardon, D. Mattimoe, D. P. McLoone, G. Noes-Holt, E. M. Pogatzki-Zahn, E. Power, B. Pradier, E. Romanos-Sirakis, A. Segelcke, R. Vinagre, J. A. Yanes, J. Zhang, X. Y. Zhang, D. P. Finn, A. S. C. Rice, Pain 162, S26-S44 (2021). 58. D. Piomelli, O. Sasso, Nat Neurosci 17, 164-174 (2014). 59. S. Pontis, A. Ribeiro, O. Sasso, D. Piomelli, Crit Rev Biochem Mol Biol 51, 7-14 (2016). 60. C. Solorzano, C. Zhu, N. Battista, G. Astarita, A. Lodola, S. Rivara, M. Mor, R. Russo, M. Maccarrone, F. Antonietti, A. Duranti, A. Tontini, S. Cuzzocrea, G. Tarzia, D. Piomelli, Proc Natl Acad Sci USA 106, 20966-20971 (2009). 61. O. Sasso, G. Moreno-Sanz, C. Martucci, N. Realini, M. Dionisi, L. Mengatto, A. Duranti, G. Tarozzo, G. Tarzia, M. Mor, R. Bertorelli, A. Reggiani, D. Piomelli, Pain 154, 350-360 (2013). 62. I. A. Khasabova, Y. Xiong, L. G. Coicou, D. Piomelli, V. Seybold, J Neurosci 32, 12735-12743 (2012). 63. Y. Fotio, O. Sasso, R. Ciccocioppo, D. Piomelli, J Pharmacol Exp Ther (2021). 64. O. Sasso, S. Pontis, A. Armirotti, G. Cardinali, D. Kovacs, M. Migliore, M. Summa, G. Moreno-Sanz, M. Picardo, D. Piomelli, Proc Natl Acad Sci USA 113, E4397-4406 (2016). 65. T. Misgeld, T. L. Schwarz, Neuron 96, 651-666 (2017). 66. M. Basan, S. Hui, H. Okano, Z. Zhang, Y. Shen, J. R. Williamson, T. Hwa, Nature 528, 99-104 (2015). 67. X. Zheng, L. Boyer, M. Jin, J. Mertens, Y. Kim, L. Ma, L. Ma, M. Hamm, F. H. Gage, T. Hunter, Elife 5, (2016). 68. G. Yellen, J Cell Biol 217, 2235-2246 (2018). 69. C. Bas-Orth, Y. W. Tan, D. Lau, H. Bading, J Biol Chem 292, 5183-5194 (2017). 70. S. C. Cheng, J. Quintin, R. A. Cramer, K. M. Shepardson, S. Saeed, V. Kumar, E. J. Giamarellos-Bourboulis, J. H. Martens, N. A. Rao, A. Aghajanirefah, G. R. Manjeri, Y. Li, D. C. Ifrim, R. J. Arts, B. M. van der Veer, P. M. Deen, C. Logie, L. A. O'Neill, P. Willems, F. L. van de Veerdonk, J. W. van der Meer, A. Ng, L. A. Joosten, C. Wijmenga, H. G. Stunnenberg, R. J. Xavier, M. G. Netea, Science 345, 1250684 (2014). 71. M. G. Netea, L. A. Joosten, E. Latz, K. H. Mills, G. Natoli, H. G. Stunnenberg, L. A. O'Neill, R. J. Xavier, Science 352, aaf1098 (2016). 72. K. E. Inyang, T. Szabo-Pardi, E. Wentworth, T. A. McDougal, G. Dussor, M. D. Burton, T. J. Price, Pharmacol Res 139, 1-16 (2019). 73. D. G. Hardie, Int J Obes (Lond) 32 Suppl 4, S7-12 (2008). 74. B. O. Mancarci, L. Toker, S. J. Tripathy, B. Li, B. Rocco, E. Sibille, P. Pavlidis, eNeuro 4, (2017). 75. R. R. Ji, C. R. Donnelly, M. Nedergaard, Nat Rev Neurosci 20, 667-685 (2019). 76. K. Inoue, M. Tsuda, Nat Rev Neurosci 19, 138-152 (2018). 77. M. L. Reitsma, J. E. Tranmer, D. M. Buchanan, E. G. Vandenkerkhof, Chronic Dis Inj Can 31, 157-164 (2011). 78. X. Yu, A. Basbaum, Z. Guan, Pain Rep 6, e883 (2021). 79. X. Li, Q. Guo, Z. Ye, E. Wang, W. Zou, Z. Sun, Z. He, T. Zhong, Y. Weng, Y. Pan, Front Neurosci 15, 620525 (2021). 80. C. J. Woolf, M. S. Chong, Anesth Analg 77, 362-379 (1993). 81. S. Pontis, F. Palese, M. Summa, N. Realini, M. Lanfranco, C. De Mei, D. Piomelli, Pharmacol Res 160, 105064 (2020). 82. O. Fiehn, G. Wohlgemuth, M. Scholz, T. Kind, D. Y. Lee, Y. Lu, S. Moon, B. Nikolau, Plant J 53, 691-704 (2008). 83. T. Kind, G. Wohlgemuth, D. Y. Lee, Y. Lu, M. Palazoglu, S. Shahbaz, O. Fiehn, Anal Chem 81, 10038-10048 (2009). 84. S. Gaetani, F. Oveisi, D. Piomelli, Neuropsychopharmacology 28, 1311-1316 (2003). 85. E. Navratilova, J. Y. Xie, T. King, F. Porreca, Ann N Y Acad Sci 1282, 1-11 (2013). 86. J. L. R. Andersson, S. N. Sotiropoulos, Neuroimage 125, 1063-1078 (2016). 87. K. Richards, C. Watson, R. F. Buckley, N. D. Kurniawan, Z. Yang, M. D. Keller, R. Beare, P. F. Bartlett, G. F. Egan, G. J. Galloway, G. Paxinos, S. Petrou, D. C. Reutens, Neuroimage 58, 732-740 (2011).

1. A method of preventing chronic pain in a subject with acute pain, the method comprising administering an effective amount of an agent to the subject, wherein said agent is administered between about 1 day and 7 days after a traumatic pain event and wherein said agent is a NAAA inhibitor, FAAH inhibitor, PPARα agonist, PEA, acetyl-L-carnitine, α-lipoic acid, or olesoxime.

2. The method of claim 1, further comprising administering said agent between about 8 days and 30 days after the traumatic pain event.

3. (canceled)

4. The method of claim 1, wherein the traumatic pain event is due to physical injury, invasive surgery, or acute illness.

5.-7. (canceled)

8. The method of claim 4, wherein the invasive surgery is knee arthroplasty, hip replacement surgery, mastectomy, open-heart surgery, hernia repair, thoracotomy, caesarian section, amputation, or open cholecystoctomy.

9. The method of claim 8, wherein the agent is administered perioperatively.

10. A method of preventing chronic pain in a subject, the method comprising administering perioperatively an effective amount of an agent to the subject, wherein said agent is a NAAA inhibitor, FAAH inhibitor, PPARα agonist, PEA, acetyl-L-carnitine, α-lipoic acid, or olesoxime.

11. A method of preventing chronic pain in a cancer patient, the method comprising administering an effective amount of an agent to the cancer patient, wherein said agent is a NAAA inhibitor, FAAH inhibitor, PPARα agonist, PEA, acetyl-L-carnitine, a-lipoic acid, or olesoxime.

12. The method of claim 11, further comprising administering an anti-cancer agent.

13. The method of claim 12, wherein the chronic pain is caused by the anti-cancer agent.

14. The method of claim 11, wherein the chronic pain is chronic peripheral neuropathy or allodynia.

15. (canceled)

16. A method of preventing chronic pain in a diabetic patient, the method comprising administering an effective amount of an agent to the diabetes patient, wherein said agent is a NAAA inhibitor, FAAH inhibitor, PPARα agonist, PEA, acetyl-L-carnitine, α-lipoic acid, or olesoxime.

17. (canceled)

18. (canceled)

19. A method of treating pain in a subject in need thereof, the method comprising administering an effective amount of an agent to the subject, wherein said agent is administered between about 1 day and 7 days after a traumatic pain event and wherein said agent is a NAAA inhibitor, FAAH inhibitor, PPARα agonist, PEA, acetyl-L-carnitine, a-lipoic acid, or olesoxime.

20. The method of claim 19, further comprising administering said agent between about 8 days and 30 days after the traumatic pain event.

21. (canceled)

22. The method of claim 19, wherein the traumatic pain event is due to physical injury, invasive surgery, or acute illness.

23.-25. (canceled)

26. The method of claim 22, wherein the invasive surgery is knee arthroplasty, hip replacement surgery, mastectomy, open-heart surgery, hernia repair, thoracotomy, caesarian section, amputation, or open cholecystoctomy.

27. The method of claim 26, wherein the agent is administered perioperatively.

28. The method of claim 19, wherein the subject is a cancer patient or a diabetic patient.

29. (canceled)

30. (canceled)

31. The method of claim 1, wherein the NAAA inhibitor is ARN16186, ARN077, or ARN19702.

32. The method of claim 1, wherein the FAAH inhibitor is URB597, an analog of URB 597, URB937, or an analog of URB937.

33.-35. (canceled)

36. The method claim 1, wherein the PPARα agonist is GW7647, PEA, or OEA.
